(19) United States
(12) Patent Application Publication
(10) Pub. No.: US 2025/0092035 A1
(43) Pub. Date: Mar. 20, 2025

(54) ISOCITRATE DEHYDROGENASE 1 INHIBITORS AND METHODS OF USE THEREOF

(71) Applicants: THE WISTAR INSTITUTE OF ANATOMY AND BIOLOGY, Philadelphia, PA (US); UNIVERSITY HOSPITALS CLEVELAND MEDICAL CENTER, Cleveland, OH (US); CASE WESTERN RESERVE UNIVERSITY, Cleveland, OH (US)

(72) Inventors: Joseph SALVINO, Chester Springs, PA (US); Farheen MOHAMMED, Philadelphia, PA (US); Joel CASSEL, Philadelphia, PA (US); Valli YELLAMELLI, Philadelphia, PA (US); Jordan M. WINTER, Shaker Heights, OH (US)

(73) Assignees: THE WISTAR INSTITUTE OF ANATOMY AND BIOLOGY, Philadelphia, PA (US); UNIVERSITY HOSPITALS CLEVELAND MEDICAL CENTER, Cleveland, OH (US); CASE WESTERN RESERVE UNIVERSITY, Cleveland, OH (US)

(21) Appl. No.: 18/728,949

(22) PCT Filed: Jan. 25, 2023
(86) PCT No.: PCT/US2023/061241

§ 371 Date: Jul. 15, 2024

Related U.S. Application Data
(60) Provisional application No. 63/302,858, filed on Jan. 25, 2022.

(51) Int. Cl. C07D 471/04; A61K 31/437; A61K 45/06; A61P 35/00

(52) U.S. Cl. CPC C07D 471/04; A61K 31/437; A61K 45/06; A61P 35/00

(57) ABSTRACT

The present disclosure provides compounds of the formula: wherein the variables are defined herein, as well as pharmaceutical compositions thereof. The present disclosure also provides methods of inhibiting isocitrate dehydrogenase 1 (IDH1) and methods of treating or preventing a disease or disorder using said compounds and/or compositions.

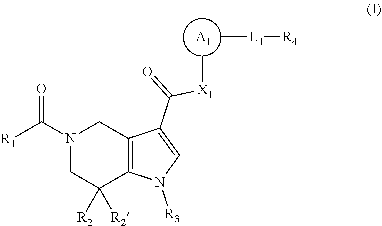

This Application claims the benefit of priority to U.S. Provisional Application No. 63/302,858, filed on Jan. 25, 2022, the entire contents of which are hereby incorporated by reference.

### BACKGROUND

This invention was made with government support under R37 CA227865 and CA010815 awarded by the National Institutes of Health. The government has certain rights in the invention.

### INCORPORATION OF SEQUENCE LISTING

This application contains a Sequence Listing XML, which has been submitted electronically and is hereby incorporated by reference in its entirety. Said XML Sequence Listing, created on Jan. 25, 2023, is named WISTP0006WO.xml and is 11,198 bytes in size.

### 1. Field

The present disclosure relates generally to the field of medicinal chemistry, pharmacology, and medicine. More particularly, it concerns methods using small molecule isocitrate dehydrogenase 1 (IDH1) inhibitors for the treatment of a disease or disorder, such as cancer.

### 2. Description of Related Art

Pancreatic cancer (PC) cells adapt to austere conditions that created by dense and hypovascular stromal tissue in the tumor microenvironment (TME) (1-5). These same adaptive survival pathways protect PC cells against chemotherapy (6). Thus, the best available treatments against PC (i.e., chemotherapy) are less effective under tumor-associated conditions. Investigative pursuits that identify metabolic dependencies in metastatic and primary PC cells (1, 7-11) should instead reveal much more compelling therapeutic alternatives that attack biologic vulnerabilities within the context of the tumor microenvironment. Such approaches offer a natural therapeutic window, since metabolic vulnerabilities in nutrient-deprived cancer cells are likely less important to well-perfused, unstressed normal tissues. Examples of relevant biologic processes utilized by PC cells to overcome nutrient limitation include autophagy (12), macropinocytosis (3, 13), and the utilization of secreted alanine from pancreatic stellate cells (14). A growing body of evidence also shows that mitochondrial function and antioxidant defense are crucial under low nutrient conditions. When energy substrates are scarce, oxidative phosphorylation is especially important to maximize ATP generation (15, 16). Nutrient limitation is also highly oxidative since glucose serves as a fuel for NADPH synthesis (17-19). In their previous studies of an RNA binding protein, HuR (ELAVL1), the inventors observed that in response to acute nutrient withdrawal, HuR promoted both antioxidant defense and mitochondrial function. Further, they determined that HuR accomplishes this in part through the post-transcriptional stabilization of wild-type isocitrate dehydrogenase 1 (wtIDH1). Out of 40 enzymes directly involved in antioxidant defense, only IDH1 expression was lost in multiple HuR-knockout cell lines, pointing to an HuR-IDH1 regulatory axis as a key component of the acute antioxidant response to stress (2, 20). Additional studies identified the regulatory HuR binding site on the IDH1 3′-UTR (6).

IDH1 is a cytosolic enzyme that catalyzes the reversible conversion of isocitrate and alpha-ketoglutarate (αKG). The reaction uses NADP+ or NADPH as a cofactor, depending on the direction of the reaction (21-23). There have been surprisingly few studies focused on the role of wtIDH1 in cancer cell survival and tumor growth (6, 21-26), and most of these studies never considered the impact of nutrient limitation on wtIDH1 dependence in cancer cells (6). However, nutrient limitation is a known feature of tumors like PC (3) and glucose is believed to be even more limiting than oxygen within the TME (27). Currently, there are limited option to address overexpression of this enzyme that maybe used to treat these cancer sub-types. Therefore, there remains a need to develop additional isocitrate dehydrogenase I inhibitors.

### SUMMARY

In one aspect, the present disclosure provides compounds of the formula:

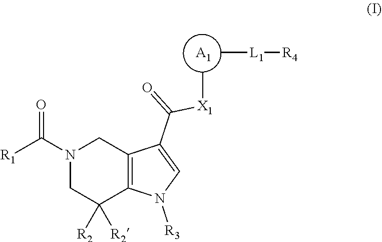

wherein:
   A₁ is arenediyl_{(C≤12)}, substituted arenediyl_{(C≤12)}, heteroarenediyl_{(C≤12)}, or substituted heteroarenediyl_{(C≤12)};
   L₁ is a covalent bond, —O—, —S—, —NR_{b}—, —C(O)—, —OC(O)—, —C(O)O—, —C(O)NR_{b}—, —NR_{b}C(O)—, —OC(O)NR_{b}—, —NR_{b}C(O)O—, —OC(O)O—, —NR_{b}C(O)NR_{b}—, -heteroarenediyl_{(C≤12)}-alkanediyl_{(C≤12)}-, or substituted -heteroarenediyl_{(C≤12)}-alkanediyl_{(C≤12)}-; wherein R_{b} is hydrogen, alkyl_{(C≤12)}, or substituted alkyl_{(C≤12)};
   R₁ is aryl_{(C≤12)}, substituted aryl_{(C≤12)}, heteroaryl_{(C≤12)}, or substituted heteroaryl_{(C≤12)};
   R₂ and R₂′ are each independently alkyl_{(C≤12)} or substituted alkyl_{(C≤12)};
   R₃ is aralkyl_{(C≤12)}, substituted aralkyl_{(C≤12)}, heteroaralkyl_{(C≤12)}, or substituted heteroaralkyl_{(C≤12)};
   R₄ is hydrogen, amino, cyano, halo, hydroxy, or nitro; or
      alkyl_{(C≤12)}, cycloalkyl_{(C≤12)}, alkenyl_{(C≤12)}, alkynyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino_{(C≤12)}, amido_{(C≤12)}, or a substituted version of any of these groups; or
      -L₂-alkanediyl_{(C≤12)}-L₃-R₅, wherein:
         L₂ and L₃ is —C(O)—, —OC(O)—, —C(O)O—, —NRₐC(O)—, —NRₐC(O)—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein:
         Rₐ and Rₐ′ are each independently hydrogen, alkyl_{(C≤12)}, or substituted alkyl_{(C≤12)};
         R₅ is a ubiquitin ligase ligand or a fluorophore; and
   X₁ is —O— or —NR_{b}—, wherein:
      R_{b} is hydrogen, alkyl_{(C≤12)}, or substituted alkyl_{(C≤12)};
or a pharmaceutically acceptable salt thereof.

In some embodiments, the compounds are further defined by the formula:

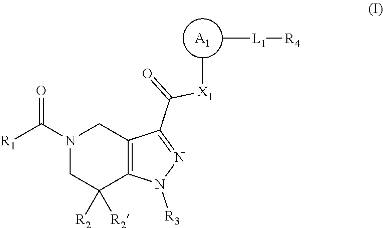

wherein:
   A₁ is arenediyl_{(C≤12)}, substituted arenediyl_{(C≤12)}, heteroarenediyl_{(C≤12)}, or substituted heteroarenediyl_{(C≤12)};
   L₁ is a covalent bond, —O—, —C(O)—, -heteroarenediyl_{(C≤12)}-alkanediyl_{(C≤12)}-, or substituted -heteroarenediyl_{(C≤12)}-alkanediyl_{(C≤12)}-;
   R₁ is aryl_{(C≤12)}, substituted aryl_{(C≤12)}, heteroaryl_{(C≤12)}, or substituted heteroaryl_{(C≤12)};
   R₂ and R₂′ are each independently alkyl_{(C≤12)} or substituted alkyl_{(C≤12)};
   R₃ is aralkyl_{(C≤12)}, substituted aralkyl_{(C≤12)}, heteroaralkyl_{(C≤12)}, or substituted heteroaralkyl_{(C≤12)};
   R₄ is hydrogen, amino, cyano, halo, hydroxy, or nitro; or
      alkyl_{(C≤12)}, cycloalkyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino_{(C≤12)}, amido_{(C≤12)}, or a substituted version of any of these groups; or
      -L₂-alkanediyl_{(C≤12)}-L₃-R₅, wherein
         L₂ and L₃ is —C(O)—, —OC(O)—, —C(O)O—, —NRₐC(O)—, —NRₐC(O)—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein:
         Rₐ and Rₐ′ are each independently hydrogen, alkyl_{(C≤12)}, or substituted alkyl_{(C≤12)};
         R₅ is a ubiquitin ligase ligand or a fluorophore; and
   X₁ is —O— or —NR_{b}—, wherein:
      R_{b} is hydrogen, alkyl_{(C≤12)}, or substituted alkyl_{(C≤12)};
or a pharmaceutically acceptable salt thereof.

In some embodiments, the compounds are further defined as:

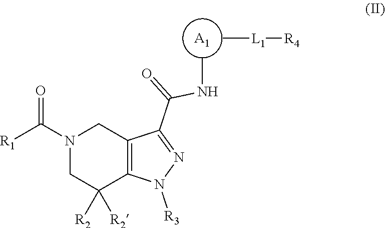

wherein:
   A₁ is arenediyl_{(C≤12)}, substituted arenediyl_{(C≤12)}, heteroarenediyl_{(C≤12)}, or substituted heteroarenediyl_{(C≤12)};
   L₁ is a covalent bond, —O—, —S—, —NR_{b}—, —C(O)—, —OC(O)—, —C(O)O—, —C(O)NR_{b}—, —NR_{b}C(O)—, —OC(O)NR_{b}—, —NR_{b}C(O)O—, —OC(O)O—, —NR_{b}C(O)NR_{b}—, -heteroarenediyl_{(C≤12)}-alkanediyl_{(C≤12)}-, or substituted -heteroarenediyl_{(C≤12)}-alkanediyl_{(C≤12)}-; wherein R_{b} is hydrogen, alkyl_{(C≤12)}, or substituted alkyl_{(C≤12)};
   R₁ is aryl_{(C≤12)}, substituted aryl_{(C≤12)}, heteroaryl_{(C≤12)}, or substituted heteroaryl_{(C≤12)};
   R₂ and R₂′ are each independently alkyl_{(C≤12)} or substituted alkyl_{(C≤12)};
   R₃ is aralkyl_{(C≤12)}, substituted aralkyl_{(C≤12)}, heteroaralkyl_{(C≤12)}, or substituted heteroaralkyl_{(C≤12)}; and
   R₄ is hydrogen, amino, cyano, halo, hydroxy, or nitro; or
      alkyl_{(C≤12)}, cycloalkyl_{(C≤12)}, alkenyl_{(C≤12)}, alkynyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino _{(C≤12)}, amido_{(C≤12)}, or a substituted version of any of these groups; or
      -L₂-alkanediyl_{(C≤12)}-L₃-R₅, wherein:
         L₂ and L₃ is —C(O)—, —OC(O)—, —C(O)O—, —NRₐC(O)—, —NRₐC(O)—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein:
         Rₐ and Rₐ′ are each independently hydrogen, alkyl_{(C≤12)}, or substituted alkyl_{(C≤12)};
         R₅ is a ubiquitin ligase ligand or a fluorophore; and
or a pharmaceutically acceptable salt thereof.

In some embodiments, the compounds are further defined as:

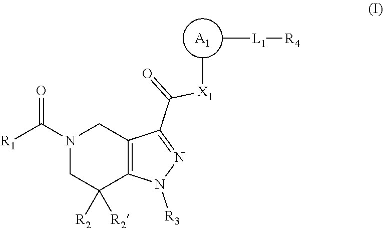

wherein:
   A₁ is arenediyl_{(C≤12)}, substituted arenediyl_{(C≤12)}, heteroarenediyl_{(C≤12)}, or substituted heteroarenediyl_{(C≤12)};
   L₁ is a covalent bond, —O—, or —C(O)—;
   R₁ is aryl_{(C≤12)}, substituted aryl_{(C≤12)}, heteroaryl_{(C≤12)}, or substituted heteroaryl_{(C≤12)};
   R₂ and R₂′ are each independently alkyl_{(C≤12)} or substituted alkyl_{(C≤12)};
   R₃ is aralkyl_{(C≤12)}, substituted aralkyl_{(C≤12)}, heteroaralkyl_{(C≤12)}, or substituted heteroaralkyl_{(C≤12)};
   R₄ is hydrogen, amino, cyano, halo, hydroxy, or nitro; or
      alkyl_{(C≤12)}, cycloalkyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino _{(C≤12)}, amido_{(C≤12)}, or a substituted version of any of these groups; or
      -alkanediyl_{(C≤12)}-L₂-R₅, wherein:
         L₂ is —C(O)—, —NRₐC(O)-alkanediyl_{(C≤6)}—O—, or substituted —NRₐC(O)-alkanediyl_{(C≤6)}—O—, wherein:
         Rₐ is hydrogen, alkyl_{(C≤12)}, or substituted alkyl_{(C≤12)};
         R₅ is a ubiquitin ligase ligand; and
   X₁ is —O— or —NR_{b}—, wherein:
      R_{b} is hydrogen, alkyl_{(C≤12)}, or substituted alkyl_{(C≤12)};
or a pharmaceutically acceptable salt thereof.
the compound is further defined as:

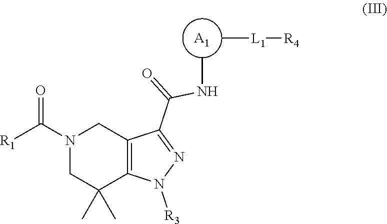

wherein:
   A₁ is arenediyl_{(C≤12)}, substituted arenediyl_{(C≤12)}, heteroarenediyl_{(C≤12)}, or substituted heteroarenediyl_{(C≤12)};
   L₁ is a covalent bond, —O—, —S—, —NR_{b}—, —C(O)—, —OC(O)—, —C(O)O—, —C(O)NR_{b}—, —NR_{b}C(O)—, —OC(O)NR_{b}—, —NR_{b}C(O)O—, —OC(O)O—,
   —NR_{b}C(O)NR_{b}—, -heteroarenediyl_{(C≤12)}-alkanediyl_{(C≤12)}-, or substituted-heteroarenediyl_{(C≤12)}-alkanediyl_{(C≤12)}-; wherein R_{b} is hydrogen, alkyl_{(C≤12)}, or substituted alkyl_{(C≤12)};
   R₁ is aryl_{(C≤12)}, substituted aryl_{(C≤12)}, heteroaryl_{(C≤12)}, or substituted heteroaryl_{(C≤12)};
   R₃ is aralkyl_{(C≤12)}, substituted aralkyl_{(C≤12)}, heteroaralkyl_{(C≤12)}, or substituted heteroaralkyl_{(C≤12)}; and
   R₄ is hydrogen, amino, cyano, halo, hydroxy, or nitro; or
      alkyl_{(C≤12)}, cycloalkyl_{(C≤12)}, alkenyl_{(C≤12)}, alkynyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino _{(C≤12)}, amido_{(C≤12)}, or a substituted version of any of these groups; or
      -L₂-alkanediyl_{(C≤12)}-L₃-R₅, wherein:
      L₂ and L₃ is —C(O)—, —OC(O)—, —C(O)O—, —NRₐC(O)—, —C(O)NRₐ—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein:
      Rₐ and Rₐ′ are each independently hydrogen, alkyl_{(C≤12)}, or substituted alkyl_{(C≤12)};
      R₅ is a ubiquitin ligase ligand or a fluorophore; and
or a pharmaceutically acceptable salt thereof.

In some embodiments, the compound is further defined as:

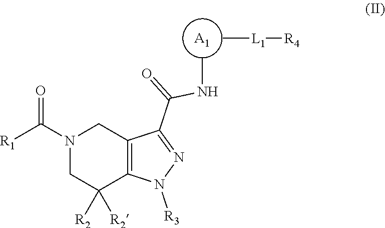

wherein:
   A₁ is arenediyl_{(C≤12)}, substituted arenediyl_{(C≤12)}, heteroarenediyl_{(C≤12)}, or substituted heteroarenediyl_{(C≤12)};
   L₁ is a covalent bond, —O—, —C(O)—, -heteroarenediyl_{(C≤12)}-alkanediyl_{(C≤12)}-, or substituted -heteroarenediyl_{(C≤12)}-alkanediyl_{(C≤12)}-;
   R₁ is aryl_{(C≤12)}, substituted aryl_{(C≤12)}, heteroaryl_{(C≤12)}, or substituted heteroaryl_{(C≤12)};
   R₂ and R₂′ are each independently alkyl_{(C≤12)} or substituted alkyl_{(C≤12)};
   R₃ is aralkyl_{(C≤12)}, substituted aralkyl_{(C≤12)}, heteroaralkyl_{(C≤12)}, or substituted heteroaralkyl_{(C≤12)}; and
   R₄ is hydrogen, amino, cyano, halo, hydroxy, or nitro; or
      alkyl_{(C≤12)}, cycloalkyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino _{(C≤12)}, amido_{(C≤12)}, or a substituted version of any of these groups; or
      -L₂-alkanediyl_{(C≤12)}-L₃-R₅, wherein:
         L₂ and L₃ is —C(O)—, —OC(O)—, —C(O)O—, —NRₐC(O)—, —NRₐC(O)—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein:
         Rₐ and Rₐ′ are each independently hydrogen, alkyl_{(C≤12)}, or substituted alkyl_{(C≤12)};
         R₅ is a ubiquitin ligase ligand or a fluorophore; and
or a pharmaceutically acceptable salt thereof.

In some embodiments, the compound is further defined as:

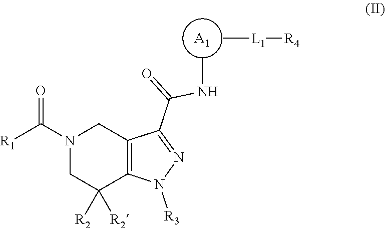

wherein:
   A₁ is arenediyl_{(C≤12)}, substituted arenediyl_{(C≤12)}, heteroarenediyl_{(C≤12)}, or substituted heteroarenediyl_{(C≤12)};
   L₁ is a covalent bond, —O—, or —C(O)—;
   R₁ is aryl_{(C≤12)}, substituted aryl_{(C≤12)}, heteroaryl_{(C≤12)}, or substituted heteroaryl_{(C≤12)};
   R₂ and R₂′ are each independently alkyl_{(C≤12)} or substituted alkyl_{(C≤12)};
   R₃ is aralkyl_{(C≤12)}, substituted aralkyl_{(C≤12)}, heteroaralkyl_{(C≤12)}, or substituted heteroaralkyl_{(C≤12)}; and
   R₄ is hydrogen, amino, cyano, halo, hydroxy, or nitro; or
      alkyl_{(C≤12)}, cycloalkyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino _{(C≤12)}, amido_{(C≤12)}, or a substituted version of any of these groups; or
      -alkanediyl_{(C≤12)}-L₂-R₅, wherein:
         L₂ is —C(O)—, —NRₐC(O)-alkanediyl_{(C≤6)}—O—, or substituted —NRₐC(O)-alkanediyl_{(C≤6)}—O—, wherein:
         Rₐ is hydrogen, alkyl_{(C≤12)}, or substituted alkyl_{(C≤12)};
         R₅ is a ubiquitin ligase ligand;
or a pharmaceutically acceptable salt thereof.

In some embodiments, the compound is further defined as:

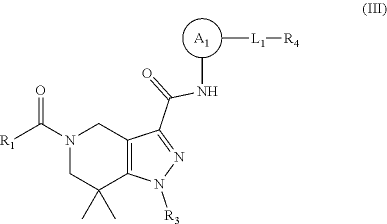

wherein:
   A₁ is arenediyl_{(C≤12)}, substituted arenediyl_{(C≤12)}, heteroarenediyl_{(C≤12)}, or substituted heteroarenediyl_{(C≤12)};
   L₁ is a covalent bond, —O—, —C(O)—, -heteroarenediyl_{(C≤12)}-alkanediyl_{(C≤12)}-, or substituted-heteroarenediyl_{(C≤12)}-alkanediyl_{(C≤12)}-;
   R₁ is aryl_{(C≤12)}, substituted aryl_{(C≤12)}, heteroaryl_{(C≤12)}, or substituted heteroaryl_{(C≤12)};
   R₃ is aralkyl_{(C≤12)}, substituted aralkyl_{(C≤12)}, heteroaralkyl_{(C≤12)}, or substituted heteroaralkyl_{(C≤12)}; and
   R₄ is hydrogen, amino, cyano, halo, hydroxy, or nitro; or
      alkyl_{(C≤12)}, cycloalkyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino _{(C≤12)}, amido_{(C≤12)}, or a substituted version of any of these groups; or
      -L₂-alkanediyl_{(C≤12)}-L₃-R₅, wherein:
         L₂ and L₃ is —C(O)—, —OC(O)—, —C(O)O—, —NRₐC(O)—, —C(O)NRₐ—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein:
         Rₐ and Rₐ′ are each independently hydrogen, alkyl_{(C≤12)}, or substituted alkyl_{(C≤12)};
      R₅ is a ubiquitin ligase ligand or a fluorophore; and
or a pharmaceutically acceptable salt thereof.

In some embodiments, the compound is further defined as:

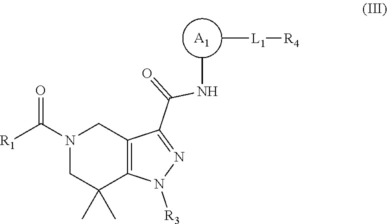

wherein:
   A₁ is arenediyl_{(C≤12)}, substituted arenediyl_{(C≤12)}, heteroarenediyl_{(C≤12)}, or substituted heteroarenediyl_{(C≤12)};
   L₁ is a covalent bond, —O—, or —C(O)—;
   R₁ is aryl_{(C≤12)}, substituted aryl_{(C≤12)}, heteroaryl_{(C≤12)}, or substituted heteroaryl_{(C≤12)};
   R₃ is aralkyl_{(C≤12)}, substituted aralkyl_{(C≤12)}, heteroaralkyl_{(C≤12)}, or substituted heteroaralkyl_{(C≤12)}; and
   R₄ is hydrogen, amino, cyano, halo, hydroxy, or nitro; or alkyl_{(C≤12)}, cycloalkyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino _{(C≤12)}, amido_{(C≤12)}, or a substituted version of any of these groups; or
      -alkanediyl_{(C≤12)}-L₂-R₅, wherein:
         L₂ is —C(O)—, —NRₐC(O)-alkanediyl_{(C≤6)}—O—, or substituted —NRₐC(O)-alkanediyl_{(C≤6)}—O—, wherein:
         Rₐ is hydrogen, alkyl_{(C≤12)}, or substituted alkyl_{(C≤12)};
         R₅ is a ubiquitin ligase ligand;
or a pharmaceutically acceptable salt thereof.

In some embodiments, the compounds are further defined as:

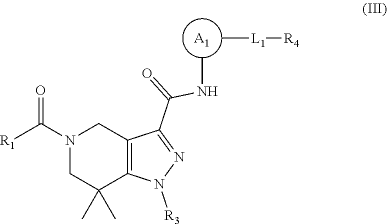

wherein:
   A₁ is arenediyl_{(C≤12)}, substituted arenediyl_{(C≤12)}, heteroarenediyl_{(C≤12)}, or substituted heteroarenediyl_{(C≤12)};
   L₁ is a covalent bond, —O—, —S—, —NR_{b}—, —C(O)—, —OC(O)—, —C(O)O—, —C(O)NR_{b}—, —NR_{b}C(O)—, —OC(O)NR_{b}—, —NR_{b}C(O)O—, —OC(O)O—, —NR_{b}C(O)NR_{b}—, -heteroarenediyl_{(C≤12)}-alkanediyl_{(C≤12)}-, or substituted -heteroarenediyl_{(C≤12)}-alkanediyl_{(C≤12)}-; wherein R_{b} is hydrogen, alkyl_{(C≤12)}, or substituted alkyl_{(C≤12)};
   R₁ is aryl_{(C≤12)}, substituted aryl_{(C≤12)}, heteroaryl_{(C≤12)}, or substituted heteroaryl_{(C≤12)};
   R₃ is aralkyl_{(C≤12)}, substituted aralkyl_{(C≤12)}, heteroaralkyl_{(C≤12)}, or substituted heteroaralkyl_{(C≤12)}; and
   R₄ is hydrogen, amino, cyano, halo, hydroxy, or nitro; or alkyl_{(C≤12)}, cycloalkyl_{(C≤12)}, alkenyl_{(C≤12)}, alkynyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino _{(C≤12)}, amido_{(C≤12)}, or a substituted version of any of these groups; or
      -L₂-alkanediyl_{(C≤12)}-L₃-R₅, wherein:
         L₂ and L₃ is —C(O)—, —OC(O)—, —C(O)O—, —NRₐC(O)—, —C(O)NRₐ—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein:
         Rₐ and Rₐ′ are each independently hydrogen, alkyl_{(C≤12)}, or substituted alkyl_{(C≤12)};
         R₅ is a ubiquitin ligase ligand or a fluorophore; and
or a pharmaceutically acceptable salt thereof.

In some embodiments, R₃ is aralkyl_{(C≤12)} or substituted aralkyl_{(C≤12)}. In some embodiments, R₃ is substituted aralkyl_{(C≤12)} such as 4-fluorobenzyl or 4-trifluoromethylbenzyl. In some embodiments, R₁ is heteroaryl_{(C≤12)} or substituted heteroaryl_{(C≤12)}. In some embodiments, R₁ is heteroaryl_{(C≤12)} such as 2-pyrrolyl, 4-pyrazolyl, 5-pyrazolyl, 2-thiophenyl, N-methylpyrrol-2-yl, or 4-methylpyrrol-2-yl.

In some embodiments, A₁ is arenediyl_{(C≤12)} or substituted arenediyl_{(C≤12)}. In some embodiments, A₁ is arenediyl_{(C≤12)} such as 1,3-benzenediyl or 1,4-benzenediyl. In other embodiments, A₁ is heteroarenediyl_{(C≤12)} or substituted heteroarenediyl_{(C≤12)}. In some embodiments, A₁ is heteroarenediyl_{(C≤12)} such as oxazol-2,4-diyl, oxazol-2,5-diyl, thiazol-2,4-diyl, or thiazol-2,5-diyl.

In some embodiments, L₁ is a covalent bond. In other embodiments, L₁ is —O—. In other embodiments, L₁ is —C(O)—. In other embodiments, L₁ is -heteroarenediyl_{(C≤12)}-alkanediyl_{(C≤12)}- or substituted -heteroarenediyl_{(C≤12)}-alkanediyl_{(C≤12)}-. In some embodiments, L₁ is -heteroarenediyl_{(C≤12)}-alkanediyl_{(C≤12)}-. In some embodiments, the heteroarenediyl_{(C≤12)} of L₁ is 1,4-triazoldiyl. In some embodiments, the alkanediyl_{(C≤12)} of L₁ is ethylene. In some embodiments, L₁ is —NR_{b}C(O)— or —C(O)NR_{b}—. In some embodiments, L₁ is —NR_{b}C(O)—. In some embodiments, R_{b} is hydrogen.

In some embodiments, R₄ is hydrogen. In other embodiments, R₄ is hydroxy. In other embodiments, R₄ is amino. In other embodiments, R₄ is halo such as fluoro. In other embodiments, R₄ is alkoxy_{(C≤12)} or substituted alkoxy_{(C≤12)}. In some embodiments, R₄ is alkoxy_{(C≤12)} such as methoxy. In other embodiments, R₄ is alkyl_{(C≤12)} or substituted alkyl_{(C≤12)}. In some embodiments, R₄ is substituted alkyl_{(C≤12)} such as 1-hydroxyethyl. In other embodiments, R₄ is cycloalkylamino(C≤12) or substituted cycloalkylamino(C≤12). In some embodiments, R₄ is cycloalkylamino(C≤12) such as cyclopropylamino, cyclopentylamino, or cyclohexylamino. In other embodiments, R₄ is alkenyl_{(C≤12)} or substituted alkenyl_{(C≤12)}. In some embodiments, R₄ is alkenyl_{(C≤12)} such as ethenyl. In other embodiments, R₄ is alkynyl_{(C≤12)} or substituted alkynyl_{(C≤12)}. In some embodiments, R₄ is alkynyl_{(C≤12)} such as propynyl.

In some embodiments, R₄ is -L₂-alkanediyl_{(C≤12)}-L₃-R₅, wherein: L₂ and L₃ is —C(O)—, —OC(O)—, —C(O)O—, —NRₐC(O)—, —C(O)NRₐ—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein: Rₐ and Rₐ′ are each independently hydrogen, alkyl_{(C≤12)}, or substituted alkyl_{(C≤12)}; and R₅ is a ubiquitin ligase ligand or a fluorophore. In some embodiments, L₂ is —C(O)NRₐ—. In some embodiments, Rₐ is hydrogen. In some embodiments, the alkanediyl_{(C≤12)} is hexanediyl. In some embodiments, L₃ is —NRₐC(S)NHR_{a′}—. In some embodiments, Rₐ or Rₐ′ is hydrogen. In some embodiments, Rₐ and Rₐ′ are both hydrogen.

In some embodiments, the compound is further defined as:

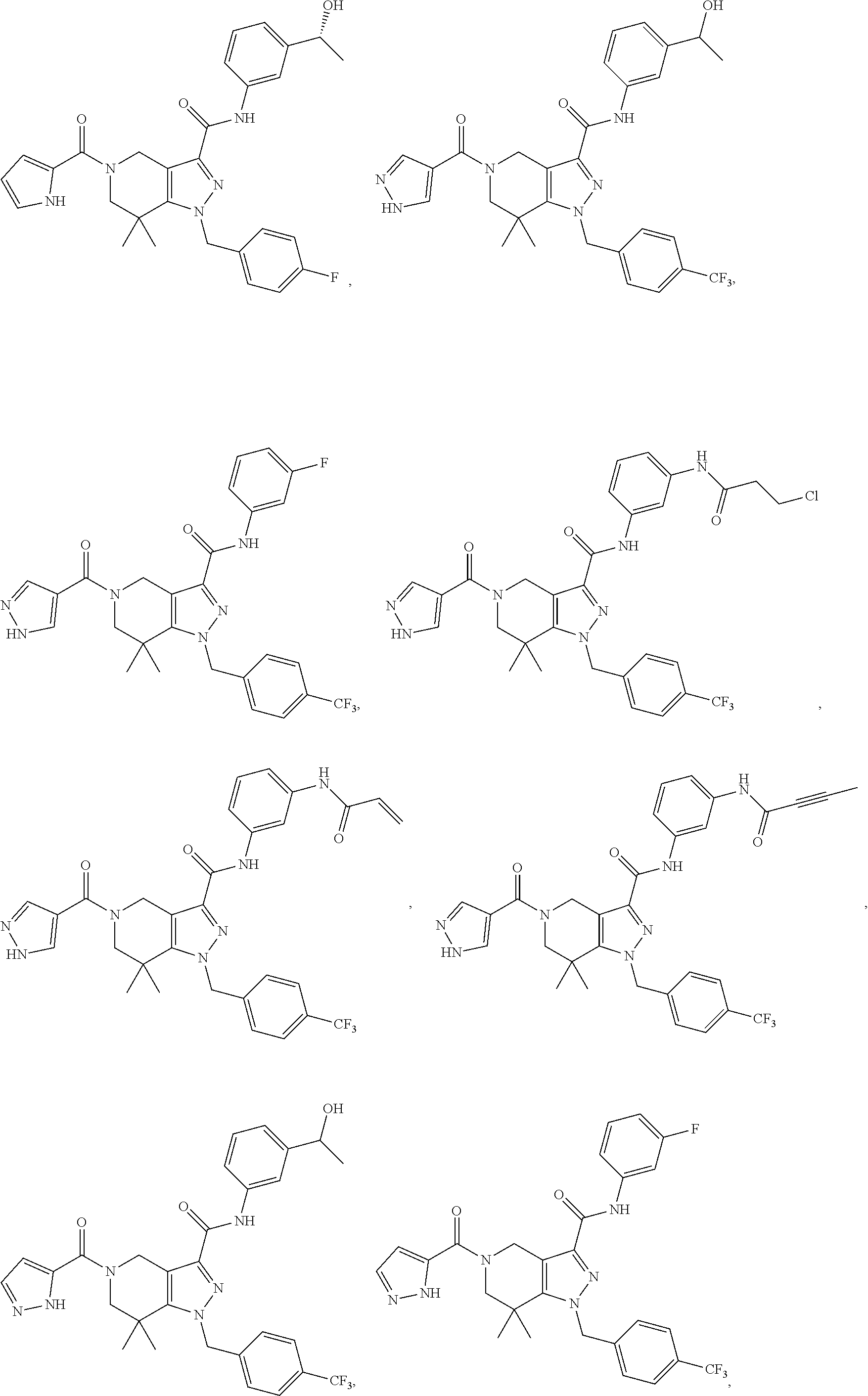

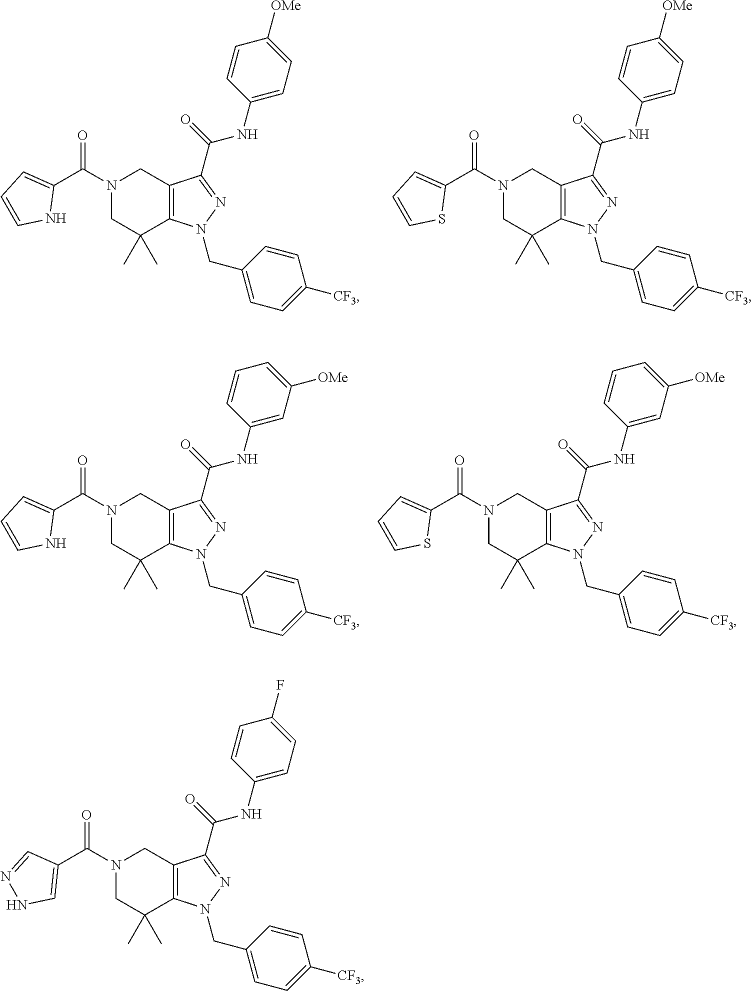 [SMILES: COc1ccc(NC(=O)c2nn(Cc3ccc(C(F)(F)F)cc3)c3c2CN(C(=O)c2ccc[nH]2)CC3(C)C)cc1 ; COc1ccc(NC(=O)c2nn(Cc3ccc(C(F)(F)F)cc3)c3c2CN(C(=O)c2cccs2)CC3(C)C)cc1 ; COc1cccc(NC(=O)c2nn(Cc3ccc(C(F)(F)F)cc3)c3c2CN(C(=O)c2ccc[nH]2)CC3(C)C)c1 ; COc1cccc(NC(=O)c2nn(Cc3ccc(C(F)(F)F)cc3)c3c2CN(C(=O)c2cccs2)CC3(C)C)c1 ; CC1(C)CN(C(=O)c2cn[nH]c2)Cc2c(C(=O)Nc3ccc(F)cc3)nn(Cc3ccc(C(F)(F)F)cc3)c21]

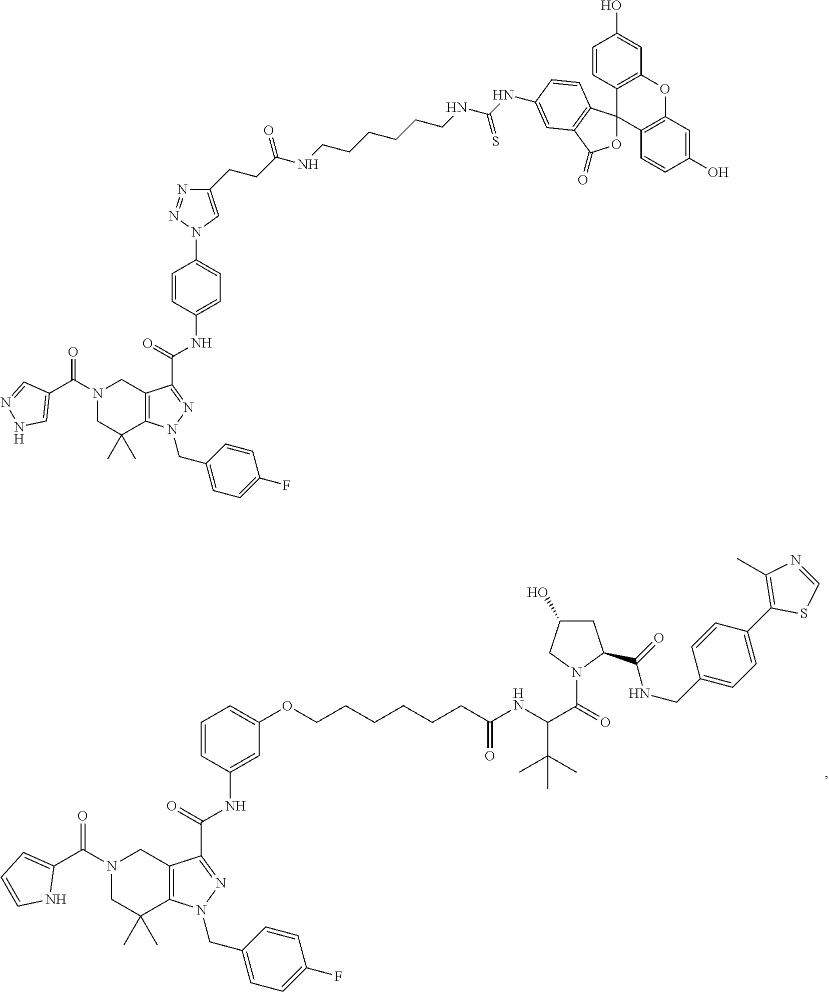 [SMILES: CC1(C)CN(C(=O)c2cn[nH]c2)Cc2c(C(=O)Nc3ccc(-n4cc(CCC(=O)NCCCCCCNC(=S)Nc5ccc6c(c5)C(=O)OC65c6ccc(O)cc6Oc6cc(O)ccc65)nn4)cc3)nn(Cc3ccc(F)cc3)c21 ; Cc1ncsc1-c1ccc(CNC(=O)[C@@H]2C[C@@H](O)CN2C(=O)C(NC(=O)CCCCCCOc2cccc(NC(=O)c3nn(Cc4ccc(F)cc4)c4c3CN(C(=O)c3ccc[nH]3)CC4(C)C)c2)C(C)(C)C)cc1]

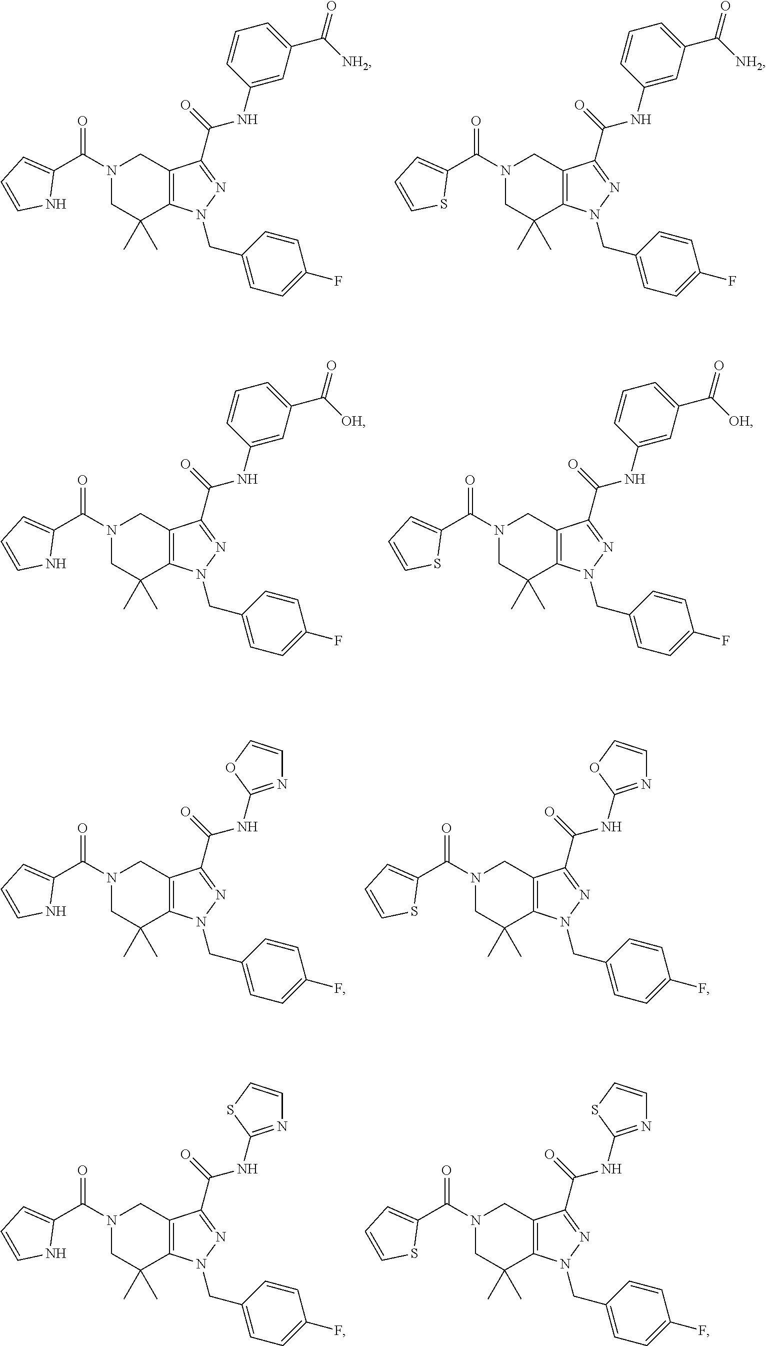

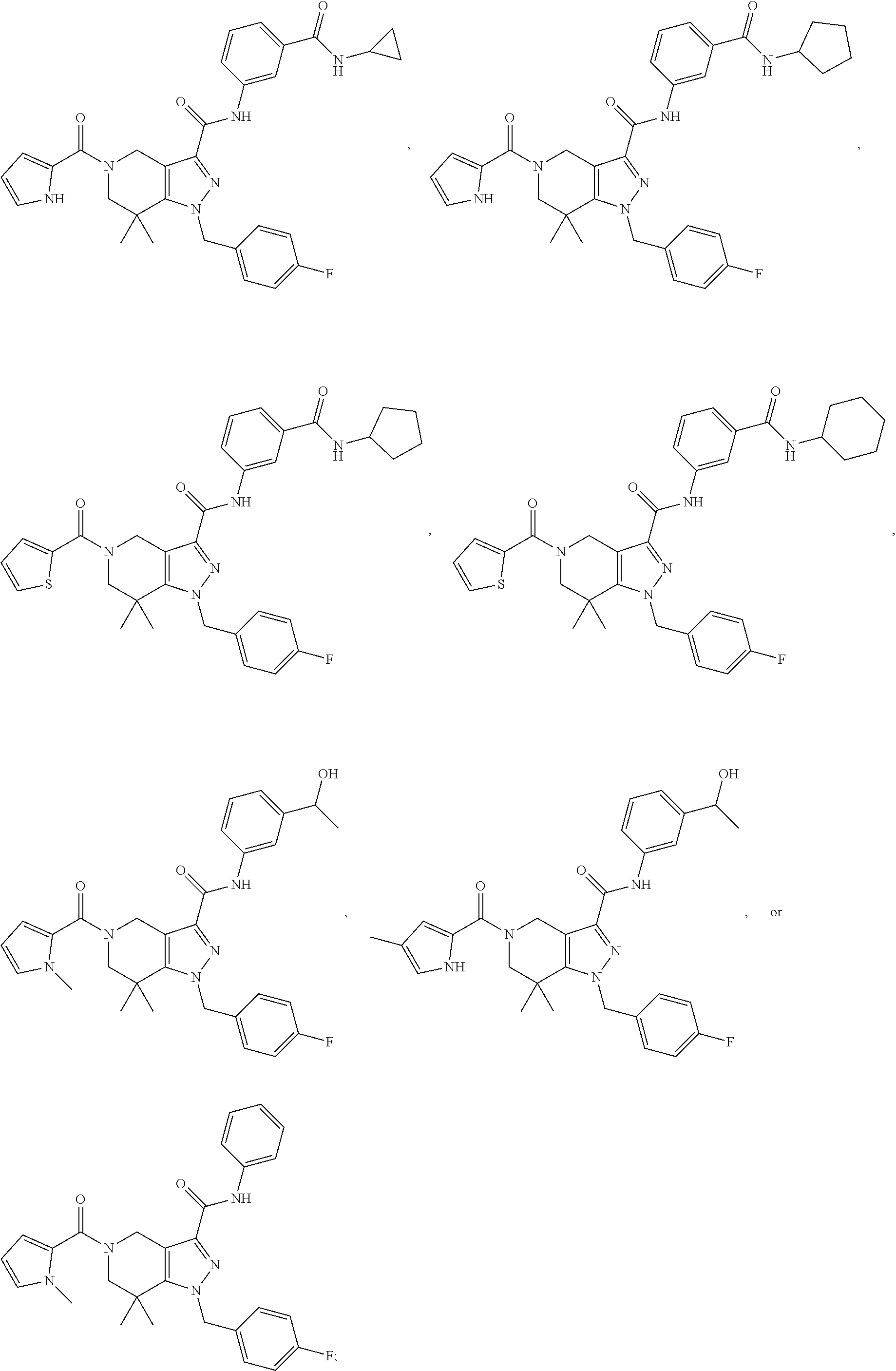

or a pharmaceutically acceptable salt thereof.

In some embodiments, the compound is further defined as:

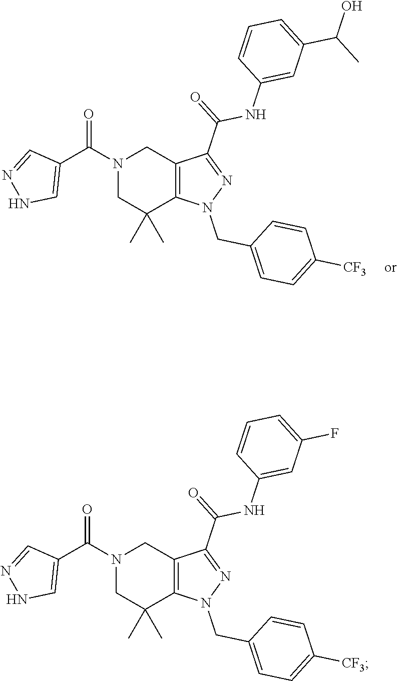 [SMILES: CC(O)c1cccc(NC(=O)c2nn(Cc3ccc(C(F)(F)F)cc3)c3c2CN(C(=O)c2cn[nH]c2)CC3(C)C)c1 ; CC1(C)CN(C(=O)c2cn[nH]c2)Cc2c(C(=O)Nc3cccc(F)c3)nn(Cc3ccc(C(F)(F)F)cc3)c21]

or a pharmaceutically acceptable salt thereof.

In another aspect, the present disclosure provides pharmaceutical composition comprising:
(a) a compound described herein; and
(b) an excipient.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the pharmaceutical composition is formulated for oral administration. In other embodiments, the pharmaceutical composition is formulated for administration via injection such as formulated for intraarterial administration, intramuscular administration, intraperitoneal administration, or intravenous administration. In some embodiments, the pharmaceutical composition is formulated as a unit dose.

In yet another aspect, the present disclosure provides methods of treating a disease or disorder in a patient in need thereof comprising administering to the patient an effective amount of a compound or composition described herein. In some embodiments, the patient is a mammal such as a human. In some embodiments, the disease or disorder is cancer. In some embodiments, the cancer is a metastatic, recurrent, or drug-resistant cancer. In some embodiments, the cells of the cancer overexpress IDH1.

In some embodiments, the methods further comprise administering to the patient a second cancer therapy such as chemotherapy, immunotherapy, radiotherapy, hormone therapy, toxin therapy, or surgery. In some embodiments, the second cancer therapy is administered at the same time as the compound or composition. In other embodiments, the second cancer therapy is administered before or after the compound or composition. In some embodiments, the methods further comprise administering to the patient a second administration of an effective amount of the compound or composition. In some embodiments, the compound or composition is administered systemically, such as intravenously, intra-arterially, orally, peritoneally, subcutaneously, or by inhalation. In some embodiments, the compound or composition is administered regionally or locally to the tumor, such as into the tumor vasculature, into a resected tumor bed, or intratumorally.

In some embodiments, the methods further comprise administering to the patient an effective amount of an alkali earth metal salt or an aqueous solution thereof. In some embodiments, the alkali earth metal salt is a magnesium (II) salt such as MgSO₄. In some embodiments, the alkali earth metal salt is administered at the same time as the compound or composition. In some embodiments, the second cancer therapy is administered before or after the compound or composition. In some embodiments, the alkali earth metal salt is administered systemically, such as intravenously, intra-arterially, orally, peritoneally, or subcutaneously. In some embodiments, the alkali earth metal salt is administered intravenously. In other embodiments, the alkali earth metal salt is administered orally.

In still another aspect, the present disclosure provides methods of inhibiting IDH1 in a cell comprising contacting the cell with a compound or composition described herein. In some embodiments, the cell is a cancer cell. In some embodiments, the cell overexpresses IDH1.

In another aspect, the present disclosure provides method of inhibiting IDH1 comprising contacting the enzyme with a compound or composition described herein. In some embodiments, the methods are performed in vitro. In other embodiments, the methods are performed in vivo. In other embodiments, the methods are performed ex vivo.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.1%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1J. IDH1 supports antioxidant defense under nutrient withdrawal in MiaPaCa-2 pancreatic cancer cells. a, ROS levels were detected by DCFDA assay under glucose withdrawal (2.5 mM) over 72 hours. 0 h indicates that cells were incubated under standard, 25 mM glucose. b, NADPH levels under the indicated conditions over 72 hours; n=4 independent experiments. c, Reductive power, as detected by an MTT assay, normalized to cell number. Cells were transiently transfected with siRNAs against different NADPH-generating enzymes and incubated under the indicated conditions for 72 hours; n=4 independent experiments. d, Relative TPM values (transcripts per million) for NADPH-generating transcripts under indicated conditions for 48 hours. Western blot of IDH1 in IDH1+/+ and IDH1−/− MiaPaCa-2 PC cells and relative NADPH levels (e), GSH/GSSH ratio (f), and ROS levels (g) under indicated conditions for 48 hours. h, Redox-related metabolites detected via LC-MS/MS in IDH1+/+ and IDH1−/− cells under glucose withdrawal for 48 hours. i, Representative data from a clonogenic growth assay of indicated cells treated with or without a glutathione precursor NAC (N-acetyl-cysteine) and reduced glutathione (GSH) at the indicated conditions. NAC treatment was given 16 hours prior to culturing cells with low glucose conditions. j, Enzymatic reaction of IDH1. Data are provided as mean±s.d. from three independent experiments, unless indicated. P values were calculated using two-tailed, unpaired Student's t-tests. *, P<0.05; **, P<0.01; ***, P<0.001.

FIGS. 2A-2L Validation of IDH1s' role in redox balance in pancreatic cancer cells. a, NADPH levels in PANC-1 PC cells under the indicated conditions for 72 hours, n=4 independent experiments. b, Reductive power, as measured by an MTT assay normalized to cell number, after transient transfection of siRNAs against NADPH-generating enzymes in PANC-1 PC cells. Cells were incubated under the indicated conditions for 72 hours. c, HuR in MiaPaCa-2 cells as positive control. d, mRNA levels associated with siRNA screening shown in FIG. 1C. e, qPCR analysis of IDH1 mRNA normalized to 18S in multiple PC cells and under 2.5 mM glucose for the indicated time interval. f, qPCR analysis of IDH1 mRNA normalized to 18S in HEK293 human embryonic kidney cells cultured under different levels of glucose for 48 hours. g, mRNA expression quantitation in MiaPaCa-2 PC cells by qPCR of IDH1, IDH2 and IDH3 transcripts. mRNA was normalized to 18S. h, Representative wells from the clonogenic growth assay from Hs766T cells. Data are provided as mean±s.d. from three independent experiments, unless indicated. P values were calculated using two-tailed, unpaired Student's t-tests. *, P<0.05; **, P<0.01; ***, P<0.001.

FIGS. 3A-311. IDH1 supports mitochondrial function under nutrient withdrawal in MiaPaCa-2 pancreatic cancer cells. a, Relative abundance of αKG in cells under low glucose conditions (2.5 mM glucose) for total of 48 hours. Oxygen consumption rate in MiaPaCa-2 PC cells under 25 mM (b) and 2.5 mM glucose (c). Cells were treated initially with αKG (4 mM) for 6 hours and then glucose was lowered to 2.5 mM glucose for 30 hours. d, Mitochondrial membrane potential detected by TMRE assay in cells under indicated conditions. e, Mitochondrial-related metabolites detected via LC-MS/MS in IDH1+/+ and IDH1−/− cells under glucose withdrawal for 48 hours. Total pool size, including glucose-independent (m+0) and glucose-dependent isotopologues (f) and isotopologue distribution (g) in cells cultured with unlabeled 2.5 mM glucose for 38 hours followed by incubating with the media containing 2.5 mM [U-¹³C]glucose for an additional 10 hours; n=4 independent experiments. (h), Representative data from a clonogenic growth assay of IDH1−/− cells treated with or without αKG (4 mM) at the indicated conditions. Data are provided as mean±s.d. from three independent experiments, unless indicated. P values were calculated using two-tailed, unpaired Student's t-tests. *, P<0.05; **, P<0.01; ***, P<0.001.

FIGS. 4A-4G IDH1−/− cells have impaired mitochondrial function. a, Representative oxygen consumption rates in MiaPaCa-2 PC cells cultured under the indicated glucose levels for 30 hours. b, ATP levels in MiaPaCa-2 PC cells cultured under the indicated conditions for 24 hours. c, Basal OCR in Hs766T PC cells cultured in 1 mM glucose for 24 hours; n=2 independent experiments. d, Mitochondrial mass in MiaPaCa-2 PC cells cultured with 2.5 mM glucose for 30 hours. e, Relative abundance of the indicated metabolites in MiaPaCa-2 PC cells cultured with unlabeled 2.5 mM glucose for 38 hours followed by incubating with unlabeled αKG (4 mM) or sodium citrate (4 mM) for 10 hours. Total pool size (e) and isotopologue distribution (f) of indicated metabolites in cells cultured with unlabeled 25 mM glucose for 38 hours followed by incubating with the media containing 25 mM [U-¹³C]glucose for an additional 10 hours; n=4 independent experiments. Data are provided as mean±s.d. from three independent experiments, unless indicated. P values were calculated using two-tailed, unpaired Student's t-tests. **, P<0.01; ***, P<0.001.

FIGS. 5A-5C. The impact of IDH1 in glutamine metabolism. a, Relative glutamine uptake was quantified via mass spectrometry in MiaPaCa-2 PC cells cultured with 4 mM [U-¹³C]glutamine under 25 or 2.5 mM glucose for 24 hours. Total pool size (b) and isotopologue distribution (c) of indicated metabolites in cells cultured with unlabeled 2.5 mM glucose and 4 mM glutamine for 38 hours followed by incubating with the media containing 2.5 mM glucose and 4 mM [U-¹³C]glutamine for an additional 10 hours. Data are provided as mean±s.d. from three independent experiments, unless indicated. P values were calculated using two-tailed, unpaired Student's t-tests. *, P<0.05; **, P<0.01; ***, P<0.001.

FIGS. 6A-6G. Impaired growth in IDH1-knockout PC cells in culture under various stresses and as xenografts. Relative growth as detected by Trypan blue assay in Hs766T cells under serum deprivation for 30 hours (a), or hydrogen peroxide under 2% serum for 72 hours (b). Growth of isogenic MiaPaCa-2 PC cells cultured in different concentrations of glucose and glutamine (c) and cultured first in 2.5 mM glucose for 24 hours, followed by CB-839 (1 μM) treatment for an additional 24 hours (d). e, Relative growth as detected by PicoGreen assay in IDH1−/− MiaPaCa-2 PC cells, transiently transfected with empty vector (EV), plasmid overexpressing catalytically active, or altered (R132H) IDH1, and treated with gemcitabine for 5 days under 5 mM glucose (relative glucose withdrawal). Growth of subcutaneous tumors from MiaPaCa-2 (f) and Hs766T (g) nude mice.

FIGS. 7A-711. IDH1 is a therapeutic target in pancreatic cancer. a, Peripheral glucose levels in mice receiving normal or 30% dextrose water. b, GC-MS analysis of intra-tumoral glucose levels (IDH1+/+vs. IDH1+/+ plus D30 water). c, Growth of subcutaneous MiaPaCa-2 tumors in mice receiving normal or 30% dextrose water. d, qPCR analysis of IDH1 transcripts normalized to 18S in xenografts shown in (a). e, Graphical depiction of 3DNA nanocarriers conjugated with IgG antibody (non-specific targeting construct) and siRNA. qPCR analysis of IDH1 mRNA transcripts (f), growth of subcutaneous tumors (g), and body weights (h) from indicated treatment arms. Data are provided as mean±s.d. For xenograft growth (B, I), error bars represent s.e.m. P values were calculated using two-tailed, unpaired Student's t-tests. **, P<0.01; ***, P<0.001.

FIGS. 8A-8G. The impact of magnesium on pancreatic cancer cell metabolism and allosteric inhibitor binding of IDH1. a, Wild-type IDH1 from the Protein Database. Mg²⁺ is believed to interact with D279 (Asp²⁷⁹) in the allosteric pocket (accession number: PDB 1t01). Under high Mg²⁺ conditions, the cation outcompetes the allosteric inhibitor, and renders the drug ineffective. Under low Mg⁺² conditions, the drug is effective. b, AG-120 effects on IDH2 activity (cell-based assay) in MiaPaCa-2 PC cells; n=2 independent experiments. c, Validation of brobe binding with wtIDH1. ATP (d) and oxygen consumption rates (e) in MiaPaCa-2 cells under indicated conditions. f, Cell viability of PC cells, cultured in MgSO₄ at the indicated concentrations and 25 mM glucose for five days. g, The impact of indicated conditions and treatments on genes involved in magnesium transport/homeostasis in MiaPaCa-2 cells. Data are provided as mean±s.d. from three independent experiments, unless indicated. P values were calculated using two-tailed, unpaired Student's t-tests. *, P<0.05; **, P<0.01; ***, P<0.001.

FIGS. 9A-9H. AG-120 is a potent wild-type IDH1 inhibitor under low Mg²⁺ conditions. a, wild-type IDH1 activity analysis in MiaPaCa-2 PC cells first cultured in media containing 0.8 or 0.08 mM MgSO₄ and 25 mM glucose for 24 hours, followed by treatment with a panel of commercial mtIDH1 inhibitors (100 nM) for 6-8 hours. Cell-free (b) and cell-based (c) IDH1 activity measurements upon incubation with AG-120 under the indicated conditions. The structure of a newly synthesized IDH1 binding probe (d) and binding affinity of probe in presence of AG-120 (e). f, Western blot analysis to evaluate thermal stability of the IDH1 protein in MiaPaCa-2 PC cells treated with vehicle or AG-120 (1 μM) in media containing 0.8 or 0.08 mM MgSO₄ for 6-8 hours. g, IDH1 activity (EC₅₀) of MiaPaCa-2 human PC cells treated with AG-120 for 24 hours under indicated conditions. h, Free magnesium levels in liver, spleen, pancreas, and MiaPaCa-2 PC xenografts vs. serum. Data are provided as mean±s.d. from two independent experiments. P values were calculated using two-tailed, unpaired Student's t-tests. **, P<0.01; ***, P<0.001.

FIGS. 10A-10G. Low glucose and Mg²⁺ levels are required for anti-cancer activity by an allosteric IDH1 inhibitor in MiaPaCa-2 cells. a, ROS levels were detected by DCFDA assay in MiaPaCa-2 PC cells treated with AG-120 (1 μM) for 48 hours under the indicated conditions, and in media containing 2.5 mM glucose. Representative oxygen consumption rate in MiaPaCa-2 PC cells treated with vehicle or AG-120 in media containing 0.8 mM Mg² and 2.5 mM glucose (b) or in media containing 0.08 mM Mg² and 2.5 mM glucose (c) for 30 hours. Total pool size, including glucose-independent (m+0) and glucose-dependent isotopologues (d) and isotopologue distribution (e) in cells cultured with unlabeled 2.5 mM glucose for 38 hours followed by incubating with the media containing 2.5 mM [U-¹³C]glucose for an additional 10 hours. (f) Relative clonogenic growth of cells treated with vehicle or AG-120 under different levels of glucose and Mg²⁺. (g) Relative clonogenic growth of indicated cells treated with vehicle or AG-120 under 2.5 mM glucose and 0.0b8 mM Mg²⁺. Data are provided as mean±s.d. from three independent experiments, unless indicated. P values were calculated using two-tailed, unpaired Student's t-tests. **, P<0.01; ***, P<0.001.

FIGS. 11A-11G. Diverse effects of AG-120 against PC cells under low glucose and low Mg²⁺ conditions. a, Total ROS levels detected in cells under indicated treatments. b, Oxidized DNA in MiaPaCa-2 PC cells detected by 8-hydroxy-deoxyguanosine (8-OHdG) levels under 2.5 mM glucose and 0.08 mM Mg² for 48 hours; n=2 independent experiments. c, Combination index analysis with indicated treatments in MiaPaCa-2 cells. d, Oxygen consumption rate in cells treated with vehicle or AG-120 in media containing 0.08 mM Mg² and 2.5 mM glucose for 30 hours. e, Isotopologue distribution in PANC-1 cells cultured with unlabeled 2.5 mM glucose for 38 hours followed by incubating with the media containing 2.5 mM [U-¹³C]glucose for an additional 10 hours. Relative clonogenic growth of PANC-1 (f) and HEK-293 (g) cells treated with vehicle or AG-120 under indicated conditions. Data are provided as mean±s.d. from three independent experiments, unless indicated. P values were calculated using two-tailed, unpaired Student's t-tests. **, P<0.01; ***, P<0.001.

FIGS. 12A-12F. Characterization of AG-120 treatment in KPC tumor model. a, Sanger sequencing of amplicons correlating with codon 132 of the wtIDH1 gene in KPC murine PC cells (K8484). The reference wildtype sequence is shown. b, IDH1 activity (EC₅₀) of KPC cells treated with AG-120 for 24 hours in media containing 0.08 mM MgSO₄. Relative clonogenic growth of KPC cells under the indicated conditions (c) and in combination with oxaliplatin under 1 mM glucose and 0.08 mM Mg² (d) and treated as indicated. e, Growth of KPC xenografts in C57BL/6J mice under indicated arms. f, CT/FDG-PET scanning and SUV values of C57BL/6J mice bearing orthotopic KPC tumors treated with vehicle or AG-120 for indicated time. Data are provided as mean±s.d. (mean±s.e.m for e) from three independent experiments. P values were calculated using two-tailed, unpaired Student's t-tests. *, P<0.05; **, P<0.01; ***, P<0.001.

FIGS. 13A-13F. AG-120 inhibits pancreatic cancer growth in mice. For all animal experiments, mice were treated orally with vehicle or AG-120 (150 mg/kg, twice daily), unless indicated. Start of treatment is shown in the graphs with an arrow. MiaPaCa-2 xenograft growth (a) and body weights (b) of nude mice treated with vehicle or AG-120 for the indicated days. (c) Ki-67 and cleaved caspase-3 staining for (a). Xenograft growth of PANC-1 (d) and PDX TM01212 (75 mg/kg, once daily, IP administration) (e). (f), Independent MiaPaCa-2 xenograft experiment in nude mice. In the indicated arm, N-acetyl-cysteine (NAC, 1.2 g/L) was administered in the drinking water for three days, followed by vehicle, AG-120, or AG-120 plus NAC. Data are provided as mean±s.e.m. P values were calculated using two-tailed, unpaired Student's t-tests. *, P<0.05; **, P<0.01; ***, P<0.001.

FIGS. 14A-14G. AG-120 impaired growth of nutrient-deprived, non-pancreatic cancers. a, Growth of subcutaneous allografts derived from murine PC (KPC cells) transplanted into nude mice. Mice were treated with vehicle or AG-120. b, αKG levels in orthotopic murine pancreatic cancer under vehicle or AG-120 treatment for 10 days. c, Survival analysis of C57BL/6J mice with orthotopic murine pancreatic cancer under vehicle or AG-120 treatment. Survival analysis (d) and tumor volumes (e) measured by ultrasound in KPC mice treated with vehicle and AG-120 for indicated days. Survival analysis (f) and tumor volumes (g) measured by MRI in tamoxifen-inducible KP−/−C mice treated with vehicle and AG-120 for indicated days. Median survival was analyzed using the Kaplan-Meier estimate and compared by the log-rank test.

FIGS. 15A-1511. Characterization of AG-120 efficacy in colorectal and lung cancer xenograft models. a, Sanger sequencing of codon 132 of the wtIDH1 gene in HCT116 colorectal and H460 lung cancer cells. b, Relative clonogenic growth of HCT116 cells treated with vehicle, AG-120 (10 μM), and GSH (4 mM) under different levels of glucose and Mg²⁺. c, Glucose levels in HCT116 Colorectal xenografts from nude mice. Growth rate of HCT116 xenograft tumors (d), and independent studies to show the impact of hyperglycemia induced by D30 (e) and when mice received high Mg water (f). g, body weights of nude mice bearing HCT116 xenografts treated as indicated. h, Relative growth of H460 subcutaneous xenografts in nude mice. Data are provided as mean±s.d. from three independent experiments (mean±s.e.m for d-h). P values were calculated using two-tailed, unpaired Student's t-tests. *, P<0.05; **, P<0.01; ***, P<0.001.

FIGS. 16A-16G. Development of a novel wild-type IDH1 inhibitor. (a), Structures of GSK321 and FSM-3-002. Cell-free analysis of wtIDH1 activity upon incubation with GSK-321 (b) and FSM-3-002 (c) under the indicated concentrations of Mg²⁺. (d-f) Growth of MiaPaCa-2 xenografts in mice treated with vehicle, GSK-321, and FSM-3-002 for indicated time. (g), Body weights of nude mice bearing MiaPaCa-2 PC xenografts treated with AG-120 or vehicle for the indicated time.

FIGS. 17A & 17B shows the ¹H NMR (FIG. 17A) and mass spectrograph of the final product (FIG. 17B).

### DESCRIPTION OF ILLUSTRATIVE EMBODIMENTS

Based on their prior studies revealing IDH1 as a regulatory target of an acute stress response regulator, HuR, and its direct role in generating NADPH and αKG, the inventors hypothesized that wtIDH1 is essential for PC cells under austere conditions. More specifically, they suspected that the products of the oxidative wtIDH1 reaction (NADPH and αKG) directly power antioxidant defense and mitochondrial function to promote PC survival. Further, targeting this enzyme offers a novel strategy to treat PC. Here, they demonstrate that compounds developed to target mutant IDH1 can be repurposed as wild-type IDH1 inhibitors. This observation is based on the scientific discovery that these drugs become potent inhibitors of the wild-type IDH1 isoenzyme in cancer cells, but only under specific conditions present in tumors: low glucose when wtIDH1 is critical for PC cell survival and low magnesium which is required for effective allosteric inhibition of the wtIDH1 isoenzyme by these compounds.

Thus, the present disclosure provides IDH1 inhibitors that may reduce the expression or activity of IDH1. The IDH1 inhibitors provided herein may be used in conjunction with an alkali earth metal salt, such as MgSO₄, to enhance inhibition. These IDH1 inhibitors may show one or more advantages over IDH1 inhibitors known in the art, including but not limited to improved efficacy, improved selectivity, or improved bioavailability. In some embodiments, the IDH1 inhibitors described herein may inhibit wild type IDH1. In other embodiments, the IDH1 inhibitors described herein may act as pan inhibitors of IDH1.

### I. Isocitrate Dehydrogenase 1 (NADP+)

Isocitrate dehydrogenase 1 (NADP+) is an enzyme that in humans is encoded by the IDH1 gene on chromosome 2. Isocitrate dehydrogenases catalyze the oxidative decarboxylation of isocitrate to 2-oxoglutarate. These enzymes belong to two distinct subclasses, one of which uses NAD⁺ as the electron acceptor and the other NADP⁺. Five isocitrate dehydrogenases have been reported: three NAD⁺-dependent isocitrate dehydrogenases, which localize to the mitochondrial matrix, and two NADP⁺-dependent isocitrate dehydrogenases, one of which is mitochondrial and the other predominantly cytosolic. Each NADP⁺-dependent isozyme is a homodimer. The protein encoded by this gene is the NADP⁺-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2,4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production. Alternatively, spliced transcript variants encoding the same protein have been found for this gene.

IDH1 is one of three isocitrate dehydrogenase isozymes, the other two being IDH2 and IDH3, and encoded by one of five isocitrate dehydrogenase genes, which are IDH1, IDH2, IDH3A, IDH3B, and IDH3G.

IDH1 forms an asymmetric homodimer in the cytoplasm and carries out its function through two hydrophilic active sites formed by both protein subunits. Each subunit or monomer is composed of three domains: a large domain (residues 1-103 and 286-414), a small domain (residues 104-136 and 186-285), and a clasp domain (residues 137 to 185). The large domain contains a Rossmann fold, while the small domain forms an a/β sandwich structure, and the clasp domain folds as two stacked double-stranded anti-parallel β-sheets. A β-sheet joins the large and small domains and is flanked by two clefts on opposite sides. The deep cleft, also known as the active site, is formed by the large and small domains of one subunit and a small domain of the other subunit. This active site includes the NADP-binding site and the isocitrate-metal ion-binding site. The shallow cleft, also referred to as the back cleft, is formed by both domains of one subunit and participates in the conformational changes of homodimeric IDH1. Finally, the clasp domains of both subunits intertwine to form a double layer of four-stranded anti-parallel β-sheets linking together the two subunits and the two active sites.

Furthermore, conformational changes to the subunits and a conserved structure at the active site affect the activity of the enzyme. In its open, inactive form, the active site structure forms a loop while one subunit adopts an asymmetric open conformation and the other adopts a quasi-open conformation. This conformation enables isocitrate to bind the active site, inducing a closed conformation that also activates IDH1. In its closed, inactive form, the active site structure becomes an α-helix that can chelate metal ions. An intermediate, semi-open form features this active site structure as a partially unraveled α-helix. There is also a type 1 peroxisomal targeting sequence at its C-terminal that targets the protein to the peroxisome.

As an isocitrate dehydrogenase, IDH1 catalyzes the reversible oxidative decarboxylation of isocitrate to yield α-ketoglutarate (α-KG) as part of the TCA cycle in glucose metabolism. This step also allows for the concomitant reduction of nicotinamide adenine dinucleotide phosphate (NADP+) to reduced nicotinamide adenine dinucleotide phosphate (NADPH). Since NADPH and α-KG function in cellular detoxification processes in response to oxidative stress, IDH1 also indirectly participates in mitigating oxidative damage. In addition, IDH1 is key to β-oxidation of unsaturated fatty acids in the peroxisomes of liver cells. IDH1 also participates in the regulation of glucose-induced insulin secretion. Notably, IDH1 is the primary producer of NADPH in most tissues, especially in brain. Within cells, IDH1 has been observed to localize to the cytoplasm, peroxisome, and endoplasmic reticulum. Under hypoxic conditions, IDH1 catalyzes the reverse reaction of α-KG to isocitrate, which contributes to citrate production via glutaminolysis. Isocitrate can also be converted into acetyl-CoA for lipid metabolism.

IDH1 mutations are heterozygous, typically involving an amino acid substitution in the active site of the enzyme in codon 132. The mutation results in a loss of normal enzymatic function and the abnormal production of 2-hydroxyglutarate (2-HG). It has been considered to take place due to a change in the binding site of the enzyme. 2-HG has been found to inhibit enzymatic function of many alpha-ketoglutarate dependent dioxygenases, including histone and DNA demethylases, causing widespread changes in histone and DNA methylation and potentially promoting tumorigenesis.

Mutations in this gene have been shown to cause metaphyseal chondromatosis with aciduria. Mutations in IDH1 are also implicated in cancer. Originally, mutations in IDH1 were detected in an integrated genomic analysis of human glioblastoma multiforme. Since then it has become clear that mutations in IDH1 and its homologue IDH2 are among the most frequent mutations in diffuse gliomas, including diffuse astrocytoma, anaplastic astrocytoma, oligodendroglioma, anaplastic oligodendroglioma, oligoastrocytoma, anaplastic oligoastrocytoma, and secondary glioblastoma. Mutations in IDH1 are often the first hit in the development of diffuse gliomas, suggesting IDH1 mutations as key events in the formation of these brain tumors. Glioblastomas with a wild-type IDH1 gene have a median overall survival of only 1 year, whereas IDH1-mutated glioblastoma patients have a median overall survival of over 2 years. In addition to being mutated in diffuse gliomas, IDH1 has also been shown to harbor mutations in human acute myeloid leukemia.

The IDH1 mutation is considered a driver alteration and occurs early during tumorigenesis, in specific in glioma and glioblastoma multiforme, its possible use as a new tumor-specific antigen to induce antitumor immunity for the cancer treatment has recently been prompted. A tumor vaccine can stimulate the body's immune system, upon exposure to a tumor-specific peptide antigen, by activation or amplification of a humoral and cytotoxic immune response targeted at the specific cancer cells.

Schumacher et al. has been shown that this attractive target (the mutation in the isocitrate dehydrogenase 1) from an immunological perspective represents a potential tumor-specific neoantigen with high uniformity and penetrance and could be exploited by immunotherapy through vaccination. Accordingly, some patients with IDH1-mutated gliomas demonstrated spontaneous peripheral CD4+ T-cell responses against the mutated IDH1 region with generation B-cell producing antibodies. Vaccination of MHC-humanized transgenic mice with mutant IDH1 peptide induced an IFN-γ CD4+T-helper 1 cell response, indicating an endogenous processing through MHC class II, and production of antibodies targeting mutant IDH1. Tumor vaccination, both prophylactic and therapeutic, resulted in growth suppression of transplanted IDH1-expressing sarcomas in MHC-humanized mice. These in vivo data show a specific and potent immunologic response in both transplanted and existing tumors.

Mutated and normal forms of IDH1 had been studied for drug inhibition both in silico and in vitro, and drugs are being developed (e.g., Ivosidenib). Ivosidenib was approved by the FDA in July 2018 for relapsed or refractory acute myeloid leukemia (AML) with an IDH1 mutation.

### II. Cancers

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, the tubulysin analogs described herein may be used to lead to decreased cell counts and as such can potentially be used to treat a variety of types of cancer lines. In some aspects, it is anticipated that the tubulysin analogs described herein may be used to treat virtually any malignancy. Here, the only requirement is the presence of LILRBs on the surface of the cancer cell, and in particular on the surface of cancer stem cells.

Cancer cells that may be treated according to the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

Particular cancers of interest are discussed in detail below.

### 1. Acute Myeloid Leukemia

Acute myeloid leukemia (AML), also known as acute myelogenous leukemia or acute nonlymphocytic leukemia (ANLL), is a cancer of the myeloid line of blood cells, characterized by the rapid growth of abnormal white blood cells that accumulate in the bone marrow and interfere with the production of normal blood cells. AML is the most common acute leukemia affecting adults, and its incidence increases with age. Although AML is a relatively rare disease, accounting for approximately 1.2% of cancer deaths in the United States, its incidence is expected to increase as the population ages.

The symptoms of AML are caused by replacement of normal bone marrow with leukemic cells, which causes a drop in red blood cells, platelets, and normal white blood cells. These symptoms include fatigue, shortness of breath, easy bruising and bleeding, and increased risk of infection. Several risk factors and chromosomal abnormalities have been identified, but the specific cause is not clear. As an acute leukemia, AML progresses rapidly and is typically fatal within weeks or months if left untreated.

AML has several subtypes; treatment and prognosis varies among subtypes. Five-year survival varies from 15-70%, and relapse rate varies from 33-78%, depending on subtype. AML is treated initially with chemotherapy aimed at inducing a remission; patients may go on to receive additional chemotherapy or a hematopoietic stem cell transplant. Recent research into the genetics of AML has resulted in the availability of tests that can predict which drug or drugs may work best for a particular patient, as well as how long that patient is likely to survive.

Most signs and symptoms of AML are caused by the replacement of normal blood cells with leukemic cells. A lack of normal white blood cell production makes the patient susceptible to infections; while the leukemic cells themselves are derived from white blood cell precursors, they have no infection-fighting capacity. A drop in red blood cell count (anemia) can cause fatigue, paleness, and shortness of breath. A lack of platelets can lead to easy bruising or bleeding with minor trauma.

The early signs of AML are often vague and nonspecific and may be similar to those of influenza or other common illnesses. Some generalized symptoms include fever, fatigue, weight loss or loss of appetite, shortness of breath, anemia, easy bruising or bleeding, petechiae (flat, pin-head sized spots under the skin caused by bleeding), bone and joint pain, and persistent or frequent infections.

Enlargement of the spleen may occur in AML, but it is typically mild and asymptomatic. Lymph node swelling is rare in AML, in contrast to acute lymphoblastic leukemia. The skin is involved about 10% of the time in the form of leukemia cutis. Rarely, Sweet's syndrome, a paraneoplastic inflammation of the skin, can occur with AML.

Some patients with AML may experience swelling of the gums because of infiltration of leukemic cells into the gum tissue. Rarely, the first sign of leukemia may be the development of a solid leukemic mass or tumor outside of the bone marrow, called a chloroma. Occasionally, a person may show no symptoms, and the leukemia may be discovered incidentally during a routine blood test.

A number of risk factors for developing AML have been identified, including: other blood disorders, chemical exposures, ionizing radiation, and genetics.

“Preleukemic” blood disorders, such as myelodysplastic syndrome or myeloproliferative disease, can evolve into AML; the exact risk depends on the type of MDS/MPS. Exposure to anticancer chemotherapy, in particular alkylating agents, can increase the risk of subsequently developing AML. The risk is highest about three to five years after chemotherapy. Other chemotherapy agents, specifically epipodophyllotoxins and anthracyclines, have also been associated with treatment-related leukemia. These treatment-related leukemias are often associated with specific chromosomal abnormalities in the leukemic cells. Occupational chemical exposure to benzene and other aromatic organic solvents is controversial as a cause of AML. Benzene and many of its derivatives are known to be carcinogenic in vitro. While some studies have suggested a link between occupational exposure to benzene and increased risk of AML, others have suggested the attributable risk, if any, is slight. High amounts of ionizing radiation exposure can increase the risk of AML. A hereditary risk for AML appears to exist. Multiple cases of AML developing in a family at a rate higher than predicted by chance alone have been reported. Several congenital conditions may increase the risk of leukemia; the most common is probably Down syndrome, which is associated with a 10- to 18-fold increase in the risk of AML.

The first clue to a diagnosis of AML is typically an abnormal result on a complete blood count. While an excess of abnormal white blood cells (leukocytosis) is a common finding, and leukemic blasts are sometimes seen, AML can also present with isolated decreases in platelets, red blood cells, or even with a low white blood cell count (leukopenia). While a presumptive diagnosis of AML can be made via examination of the peripheral blood smear when there are circulating leukemic blasts, a definitive diagnosis usually requires an adequate bone marrow aspiration and biopsy.

Marrow or blood is examined via light microscopy, as well as flow cytometry, to diagnose the presence of leukemia, to differentiate AML from other types of leukemia (e.g., acute lymphoblastic leukemia—ALL), and to classify the subtype of disease (see below). A sample of marrow or blood is typically also tested for chromosomal abnormalities by routine cytogenetics or fluorescent in situ hybridization. Genetic studies may also be performed to look for specific mutations in genes such as FMS-like tyrosine kinase 3 (FLT3), nucleophosmin, and KIT, which may influence the outcome of the disease.

Cytochemical stains on blood and bone marrow smears are helpful in the distinction of AML from ALL, and in subclassification of AML. The combination of a myeloperoxidase or Sudan black stain and a nonspecific esterase stain will provide the desired information in most cases. The myeloperoxidase or Sudan black reactions are most useful in establishing the identity of AML and distinguishing it from ALL. The nonspecific esterase stain is used to identify a monocytic component in AMLs and to distinguish a poorly differentiated monoblastic leukemia from ALL.

The diagnosis and classification of AML can be challenging and should be performed by a qualified hematopathologist or hematologist. In straightforward cases, the presence of certain morphologic features (such as Auer rods) or specific flow cytometry results can distinguish AML from other leukemias; however, in the absence of such features, diagnosis may be more difficult.

According to the widely used WHO criteria, the diagnosis of AML is established by demonstrating involvement of more than 20% of the blood and/or bone marrow by leukemic myeloblasts. The French-American-British (FAB) classification is a bit more stringent, requiring a blast percentage of at least 30% in bone marrow (BM) or peripheral blood (PB) for the diagnosis of AML. AML must be carefully differentiated from “preleukemic” conditions such as myelodysplastic or myeloproliferative syndromes, which are treated differently.

Because acute promyelocytic leukemia (APL) has the highest curability and requires a unique form of treatment, it is important to quickly establish or exclude the diagnosis of this subtype of leukemia. Fluorescent in situ hybridization performed on blood or bone marrow is often used for this purpose, as it readily identifies the chromosomal translocation [t(15;17)(g22;q12);] that characterizes APL. There is also a need to molecularly detect the presence of PML/RARA fusion protein, which is an oncogenic product of that translocation.

First-line treatment of AML consists primarily of chemotherapy and is divided into two phases: induction and post-remission (or consolidation) therapy. The goal of induction therapy is to achieve a complete remission by reducing the number of leukemic cells to an undetectable level; the goal of consolidation therapy is to eliminate any residual undetectable disease and achieve a cure. Hematopoietic stem cell transplantation is usually considered if induction chemotherapy fails or after a patient relapses, although transplantation is also sometimes used as front-line therapy for patients with high-risk disease.

All FAB subtypes except M3 are usually given induction chemotherapy with cytarabine (ara-C) and an anthracycline (most often daunorubicin). This induction chemotherapy regimen is known as “7+3” (or “3+7”), because the cytarabine is given as a continuous IV infusion for seven consecutive days while the anthracycline is given for three consecutive days as an IV push. Up to 70% of patients will achieve a remission with this protocol. Other alternative induction regimens, including high-dose cytarabine alone, FLAG-like regimens or investigational agents, may also be used. Because of the toxic effects of therapy, including myelosuppression and an increased risk of infection, induction chemotherapy may not be offered to the very elderly, and the options may include less intense chemotherapy or palliative care.

The M3 subtype of AML, also known as acute promyelocytic leukemia (APL), is almost universally treated with the drug all-trans-retinoic acid (ATRA) in addition to induction chemotherapy, usually an anthracycline. Care must be taken to prevent disseminated intravascular coagulation (DIC), complicating the treatment of APL when the promyelocytes release the contents of their granules into the peripheral circulation. APL is eminently curable, with well-documented treatment protocols.

The goal of the induction phase is to reach a complete remission. Complete remission does not mean the disease has been cured; rather, it signifies no disease can be detected with available diagnostic methods. Complete remission is obtained in about 50% 75% of newly diagnosed adults, although this may vary based on the prognostic factors described above. The length of remission depends on the prognostic features of the original leukemia. In general, all remissions will fail without additional consolidation therapy.

Even after complete remission is achieved, leukemic cells likely remain in numbers too small to be detected with current diagnostic techniques. If no further post-remission or consolidation therapy is given, almost all patients will eventually relapse. Therefore, more therapy is necessary to eliminate non-detectable disease and prevent relapse—that is, to achieve a cure.

The specific type of post-remission therapy is individualized based on a patient's prognostic factors (see above) and general health. For good-prognosis leukemias (i.e., inv(16), t(8;21), and t(15;17)), patients will typically undergo an additional three to five courses of intensive chemotherapy, known as consolidation chemotherapy. For patients at high risk of relapse (e.g., those with high-risk cytogenetics, underlying MDS, or therapy-related AML), allogeneic stem cell transplantation is usually recommended if the patient is able to tolerate a transplant and has a suitable donor. The best post-remission therapy for intermediate-risk AML (normal cytogenetics or cytogenetic changes not falling into good-risk or high-risk groups) is less clear and depends on the specific situation, including the age and overall health of the patient, the patient's personal values, and whether a suitable stem cell donor is available.

For patients who are not eligible for a stem cell transplant, immunotherapy with a combination of histamine dihydrochloride (Ceplene) and interleukin 2 (Proleukin) after the completion of consolidation has been shown to reduce the absolute relapse risk by 14%, translating to a 50% increase in the likelihood of maintained remission.

For patients with relapsed AML, the only proven potentially curative therapy is a hematopoietic stem cell transplant, if one has not already been performed. In 2000, the monoclonal antibody-linked cytotoxic agent gemtuzumab ozogamicin (Mylotarg) was approved in the United States for patients aged more than 60 years with relapsed AML who are not candidates for high-dose chemotherapy. This drug was voluntarily withdrawn from the market by its manufacturer, Pfizer in 2010. Since treatment options for relapsed AML are so limited, palliative care may be offered.

Patients with relapsed AML who are not candidates for stem cell transplantation, or who have relapsed after a stem cell transplant, may be offered treatment in a clinical trial, as conventional treatment options are limited. Agents under investigation include cytotoxic drugs such as clofarabine, as well as targeted therapies, such as farnesyl transferase inhibitors, decitabine, and inhibitors of MDR1 (multidrug-resistance protein). For relapsed acute promyelocytic leukemia (APL), arsenic trioxide has been tested in trials and approved by the U.S. FDA. Like ATRA, arsenic trioxide does not work with other subtypes of AML.

While acute myeloid leukemia is a curable disease, the chance of cure for a specific patient depends on a number of prognostic factors. The single most important prognostic factor in AML is cytogenetics, or the chromosomal structure of the leukemic cell. Certain cytogenetic abnormalities are associated with very good outcomes (for example, the (15:17) translocation in acute promyelocytic leukemia). About half of AML patients have “normal” cytogenetics; they fall into an intermediate risk group. A number of other cytogenetic abnormalities are known to associate with a poor prognosis and a high risk of relapse after treatment.

AML which arises from a pre-existing myelodysplastic syndrome (MDS) or myeloproliferative disease (so-called secondary AML) has a worse prognosis, as does treatment-related AML arising after chemotherapy for another previous malignancy. Both of these entities are associated with a high rate of unfavorable cytogenetic abnormalities.

In some studies, age >60 years and elevated lactate dehydrogenase level were also associated with poorer outcomes. As with most forms of cancer, performance status (i.e., the general physical condition and activity level of the patient) plays a major role in prognosis as well.

FLT3 internal tandem duplications (ITDs) have been shown to confer a poorer prognosis in AML. Treating these patients with more aggressive therapy, such as stem-cell transplantation in first remission, has not been shown to enhance long-term survival. ITDs of FLT3 may be associated with leukostasis. In 2012, the FLT3 inhibitor quizartinib showed positive phase II trial results in AML patients with FLT3-ITD mutations. In 2017, the FLT3 inhibitor Rydapt® (midostaurin, formerly PKC412) was approved by FDA for the treatment of newly diagnosed AML patients who are FLT3 mutation-positive (FLT3+), as detected by an FDA-approved test, in combination with chemotherapy.

Researchers are investigating the clinical significance of c-KIT mutations in AML. These are prevalent, and clinically relevant because of the availability of tyrosine kinase inhibitors, such as imatinib and sunitinib that can block the activity of c-KIT pharmacologically. Other genes being investigated as prognostic factors or therapeutic targets include CEBPA, BAALC, ERG, and NPM1.

### 2. Brain Cancers

In one aspect, the present disclosure addresses the treatment of brain cancers. A brain tumor occurs when abnormal cells form within the brain. There are two main types of tumors: malignant or cancerous tumors and benign tumors. Cancerous tumors can be divided into primary tumors, which start within the brain, and secondary tumors, which have spread from elsewhere, known as brain metastasis tumors. All types of brain tumors may produce symptoms that vary depending on the part of the brain involved. These symptoms may include headaches, seizures, problems with vision, vomiting and mental changes. The headache is classically worse in the morning and goes away with vomiting. Other symptoms may include difficulty walking, speaking or with sensations. As the disease progresses, unconsciousness may occur.

The cause of most brain tumors is unknown. Uncommon risk factors include inherited neurofibromatosis, exposure to vinyl chloride, Epstein-Barr virus and ionizing radiation. The evidence for mobile phone exposure is not clear. The most common types of primary tumors in adults are meningiomas (usually benign) and astrocytomas such as glioblastomas. In children, the most common type is a malignant medulloblastoma. Diagnosis is usually by medical examination along with computed tomography or magnetic resonance imaging. The result is then often confirmed by a biopsy. Based on the findings, the tumors are divided into different grades of severity.

Treatment may include some combination of surgery, radiation therapy and chemotherapy. Anticonvulsant medication may be needed if seizures occur. Dexamethasone and furosemide may be used to decrease swelling around the tumor. Some tumors grow gradually, requiring only monitoring and possibly needing no further intervention. Treatments that use a person's immune system are being studied. Outcome varies considerably depending on the type of tumor and how far it has spread at diagnosis. Glioblastomas usually have poor outcomes, while meningiomas usually have good outcomes. The average five-year survival rate for all brain cancers in the United States is 33%.

Secondary, or metastatic, brain tumors are more common than primary brain tumors, with about half of metastases coming from lung cancer. Primary brain tumors occur in around 250,000 people a year globally, making up less than 2% of cancers. In children younger than 15, brain tumors are second only to acute lymphoblastic leukemia as the most common form of cancer. In Australia, the average lifetime economic cost of a case of brain cancer is $1.9 million, the greatest of any type of cancer.

The brain is divided into four lobes and each lobe or area has its own function. A tumor in any of these lobes may affect the area's performance. The location of the tumor is often linked to the symptoms experienced but each person may experience something different.

Frontal lobe tumors may contribute to poor reasoning, inappropriate social behavior, personality changes, poor planning, lower inhibition, and decreased production of speech (Broca's area).

Temporal lobe tumors may contribute to poor memory, loss of hearing, difficulty in language comprehension (Wernicke's area).

Parietal lobe tumors may result in poor interpretation of languages and difficulty speaking, difficulty writing, drawing, naming, and recognizing, and poor spatial and visual perception.

Occipital lobe tumors may result in poor or loss of vision.

Cerebellum tumors may cause poor balance, muscle movement, and posture.

Brain stem tumors can cause seizures, induce endocrine problems, respiratory changes, visual changes, headaches and partial paralysis.

Human brains are surrounded by a system of connective tissue membranes called meninges that separate the brain from the skull. This three-layered covering is composed of (from the outside in) the dura mater (“hard mother”), arachnoid mater (“spidery mother”), and pia mater (“tender mother”). The arachnoid and pia are physically connected and thus often considered as a single layer, the pia-arachnoid, or leptomeninges. Between the arachnoid mater and the pia mater is the subarachnoid space which contains cerebrospinal fluid (CSF). This fluid circulates in the narrow spaces between cells and through the cavities in the brain called ventricles, to nourish, support, and protect the brain tissue. Blood vessels enter the central nervous system through the perivascular space above the pia mater. The cells in the blood vessel walls are joined tightly, forming the blood-brain barrier which protects the brain from toxins that might enter through the blood. Tumors of the meninges are meningiomas and are often benign.

The brains of humans and other vertebrates are composed of very soft tissue and have a gelatin-like texture. Living brain tissue has a pink tint in color on the outside (gray matter), and nearly complete white on the inside (white matter), with subtle variations in color. Three separate brain areas make up most of the brain's volume:
telencephalon (cerebral hemispheres or cerebrum)
mesencephalon (midbrain)
cerebellum.

These areas are composed of two broad classes of cells: neurons and glia. These two types are equally numerous in the brain as a whole, although glial cells outnumber neurons roughly 4 to 1 in the cerebral cortex. Glia come in several types, which perform a number of critical functions, including structural support, metabolic support, insulation, and guidance of development. Primary tumors of the glial cells are called gliomas and often are malignant by the time they are diagnosed.

The pons in the brainstem is a specific region that consists of myelinated axons much like the spinal cord. The thalamus and hypothalamus of the diencephalon also consist of neuron and glial cell tissue with the hypophysis (pituitary gland) and pineal gland (which is glandular tissue) attached at the bottom; tumors of the pituitary and pineal gland are often benign. The medulla oblongata is at the start of the spinal cord and is composed mainly of neuron tissue enveloped in oligodendrocytes and meninges tissue. The spinal cord is made up of bundles of these axons. Glial cells such as Schwann cells in the periphery or, within the cord itself, oligodendrocytes, wrap themselves around the axon, thus promoting faster transmission of electrical signals and also providing for general maintenance of the environment surrounding the cord, in part by shuttling different compounds around in response to injury or other stimulus.

Although there is no specific or singular symptom or sign, the presence of a combination of symptoms and the lack of corresponding indications of other causes can be an indicator for investigation towards the possibility of a brain tumor. Brain tumors have similar characteristics and obstacles when it comes to diagnosis and therapy with tumors located elsewhere in the body. However, they create specific issues that follow closely to the properties of the organ they are in.

The diagnosis will often start by taking a medical history noting medical antecedents, and current symptoms. Clinical and laboratory investigations will serve to exclude infections as the cause of the symptoms. Examinations in this stage may include the eyes, otolaryngological (or ENT) and electrophysiological exams. The use of electroencephalography (EEG) often plays a role in the diagnosis of brain tumors.

Brain tumors, when compared to tumors in other areas of the body, pose a challenge for diagnosis. Commonly, radioactive tracers are taken up in large volumes in tumors due to the high activity of tumor cells, allowing for radioactive imaging of the tumor. However, most of the brain is separated from the blood by the blood-brain barrier (BBB), a membrane which exerts a strict control over what substances are allowed to pass into the brain. Therefore, many tracers that may reach tumors in other areas of the body easily would be unable to reach brain tumors until there was a disruption of the BBB by the tumor. Disruption of the BBB is well imaged via MRI or CT scan and is therefore regarded as the main diagnostic indicator for malignant gliomas, meningiomas, and brain metastases.

Swelling or obstruction of the passage of cerebrospinal fluid (CSF) from the brain may cause (early) signs of increased intracranial pressure which translates clinically into headaches, vomiting, or an altered state of consciousness, and in children changes to the diameter of the skull and bulging of the fontanelles. More complex symptoms such as endocrine dysfunctions should alarm doctors not to exclude brain tumors.

A bilateral temporal visual field defect (due to compression of the optic chiasm) or dilation of the pupil, and the occurrence of either slowly evolving or the sudden onset of focal neurologic symptoms, such as cognitive and behavioral impairment (including impaired judgment, memory loss, lack of recognition, spatial orientation disorders), personality or emotional changes, hemiparesis, hypoesthesia, aphasia, ataxia, visual field impairment, impaired sense of smell, impaired hearing, facial paralysis, double vision, or more severe symptoms such as tremors, paralysis on one side of the body hemiplegia, or (epileptic) seizures in a patient with a negative history for epilepsy, should raise the possibility of a brain tumor.

Tumors can be benign or malignant, can occur in different parts of the brain, and may be primary or secondary. A primary tumor is one that has started in the brain, as opposed to a metastatic tumor, which is something that has spread to the brain from another part of the body. The incidence of metastatic tumors are more prevalent than primary tumors by 4:1. Tumors may or may not be symptomatic: some tumors are discovered because the patient has symptoms, others show up incidentally on an imaging scan, or at an autopsy.

The most common primary brain tumors are:
Gliomas (50.4%)
Meningiomas (20.8%)
Pituitary adenomas (15%)
Nerve sheath tumors (8%).

Other types include Anaplastic astrocytoma, Astrocytoma, Central neurocytoma, Choroid plexus carcinoma, Choroid plexus papilloma, Choroid plexus tumor, Dysembryoplastic neuroepithelial tumour, Ependymal tumor, Fibrillary astrocytoma, Giant-cell glioblastoma, Glioblastoma multiforme, Gliomatosis cerebri, Gliosarcoma, Hemangiopericytoma, Medulloblastoma, Medulloepithelioma, Meningeal carcinomatosis, Neuroblastoma, Neurocytoma, Oligoastrocytoma, Oligodendroglioma, Optic nerve sheath meningioma, Pediatric ependymoma, Pilocytic astrocytoma, Pinealoblastoma, Pineocytoma, Pleomorphic anaplastic neuroblastoma, Pleomorphic xanthoastrocytoma, Primary central nervous system lymphoma, Sphenoid wing meningioma, Subependymal giant cell astrocytoma, Subependymoma, Trilateral retinoblastoma.

A medical team generally assesses the treatment options and presented to the person affect and their family. Various types of treatment are available depending on neoplasm type and location and may be combined to give the best chances of survival (discussed in greater detail in Section IV on Combination Therapies):

Surgery: complete or partial resection of the tumor with the objective of removing as many tumor cells as possible.

Radiotherapy: the most commonly used treatment for brain tumors; the tumor is irradiated with beta, x rays or gamma rays.

Chemotherapy: is a treatment option for cancer, however, it is not always used to treat brain tumors as the blood-brain barrier can prevent some drugs from reaching the cancerous cells.

A variety of experimental therapies are available through clinical trials. Survival rates in primary brain tumors depend on the type of tumor, age, functional status of the patient, the extent of surgical tumor removal and other factors specific to each case.

Anaplastic Astrocytoma. The histologic features of anaplastic astrocytomas are similar to those of low-grade astrocytomas but these features are more abundant and exaggerated. These tumors are WHO grade III. Cellularity is more increased, as are nuclear and cellular pleomorphism. These features may be extreme, with back-to-back cells and bizarre, hyperchromatic nuclei. Cytoplasm may be scanty, with nuclear lobation and enlargement indicating anaplasia. Mitotic activity is easily recognized in most anaplastic astrocytomas but inexplicably may be absent in areas with gemistocytes.

The range of anaplasia in this grade is broad, with some examples showing low cellularity and pleomorphism with a few mitotic figures and others being highly cellular and pleomorphic with frequent mitoses, lacking only the necrosis required for a histologic diagnosis of glioblastoma. For this reason, it is useful to have a more objective indicator of behavior, and some markers of cell proliferation have been used in an attempt to predict prognosis more accurately. The most used markers in this area have been antibodies to bromodeoxyuridine (BrdU) and Ki-67. The cellular incorporation of BrdU is a specific marker of the DNA synthesis phase of the cell cycle, whereas the Ki-67 antibody labels an antigen that is present in all phases of the cell cycle except GO. Both antibodies can be identified by immunohistochemical staining in paraffin-embedded tissue sections. As a generalization, higher labeling rates for anaplastic astrocytomas is associated with poor prognosis.

Glioblastoma multiforme. Glioblastoma, also known as glioblastoma multiforme, is the glioma with the highest grade of malignancy, WHO grade IV. It represents 15% to 23% of intracranial tumors and about 50%-60% of astrocytomas. Most examples are generally considered to arise from astrocytes because glial fibrillary acidic protein can be identified in the cell cytoplasm. Some examples, however, apparently arise from other glial lineages, such as oligodendrocytes. Glioblastoma is the most frequently occurring astrocytoma. Autopsy and serial biopsy studies have shown that some astrocytomas progress through the grades of malignancy with transformation from low-grade to anaplastic astrocytoma to glioblastoma. But, because some examples of glioblastoma appear to arise rapidly in otherwise normal patients and are recognized when they are small, it is thought that this variety of glioblastoma can also arise directly from malignant transformation of astrocyte precursor cells without passing through the lower grades of malignancy.

Tumor necrosis is the characteristic gross feature that distinguishes glioblastoma from anaplastic astrocytoma. Another microscopic feature that is distinctive and diagnostic is the presence of proliferative vascular changes within the tumor. These changes may occur in the endothelial cells (vascular endothelial hyperplasia or proliferation) or in the cells of the vessel wall itself (vascular mural cell proliferation). Both types of change are sometimes considered together as microvascular proliferation. Glioblastomas cellularity is usually extremely high. The individual cells may be small, with a high nuclear:cytoplasmic ratio, or very large and bizarre, with abundant eosinophilic cytoplasm. These same small cells may appear to condense in rows around areas of tumor necrosis, forming the characteristic pseudopalisades. Glioblastoma tumors have a propensity to infiltrate the brain extensively, spreading even to distant locations and giving the appearance of a multifocal glioma. Some examples are truly multifocal (i.e., arising in multiple simultaneous primary sites) while many of these multifocal tumors show a histologic connection when the whole brain is examined at autopsy.

Oligodendrogliomas. Like astrocytomas, oligodendrogliomas mimic the histology of their presumed cell of origin. They also arise primarily in the white matter but tend to infiltrate the cerebral cortex more than do astrocytomas of a similar grade of malignancy. Like astrocytomas, grading schemes of histologic malignancy have been used for oligodendrogliomas, but these correlate less well with prognosis than those used for astrocytomas. Many of the histologic features used to grade oligodendrogliomas are similar to those used for astrocytomas: cellularity, pleomorphism, mitotic activity, vascular changes, and necrosis. Lower-grade oligodendrogliomas may have microcysts. Oligodendrogliomas of all histologic grades tend to infiltrate the cortex readily and to form clusters of neoplastic cells in the subpial region, around neurons, and around blood vessels. In general, the cells of oligodendrogliomas have round, regular nuclei and distinct cytoplasmic borders with clearing of the cytoplasm. Another fairly distinctive and diagnostically helpful feature is the vascular pattern of oligodendrogliomas, referred to as “chicken-wire” vessels that can divide the tumor into discrete lobules. With increasing anaplasia, oligodendrogliomas can become highly cellular and pleomorphic, approaching an appearance of glioblastoma multiforme with the presence of necrosis. Although it is correct to classify these as anaplastic oligodendrogliomas, some would use the term glioblastoma once necrosis is identified in any high-grade glial neoplasm. One justification for separating anaplastic oliogdendrogliomas from astrocytic glioblastomas is the slightly better prognosis of the former, even in this highest grade of malignancy. Some authors have reported that a MIB-1 labeling index of >3%-5% predicts a worse prognosis in oligodendrogliomas.

Oligoastrocytomas. Many, if not most, oligodendrogliomas occur with a regional or intimate cellular mixture of astrocytoma. For the diagnosis of mixed glioma, the proportion of each should be substantial, but authors have differing opinions with respect to exact numbers; usually a mixture with a range from 10% to 25% of the minor element is used to diagnose a mixed glioma. Oligoastrocytomas and anaplastic oligoastrocytomas correspond to WHO grade II or grade III, respectively. Histologic features of anaplasia may be present in either component and will affect the prognosis adversely. Such features include marked cellular pleomorphism, high cellularity, and a high mitotic rate. Microvascular proliferation and necrosis may also be seen. Prognosis and response to therapy have not been shown to depend on the proportion of the oligodendroglial versus the astrocytic component, although paradoxically, the BrdU LI of the oligodendroglial component is more predictive for survival than the astrocytic component and far advanced tumor progressions are dominated by the astrocytic component.

### III. Compounds of the Present Disclosure

The compounds of the present invention (also referred to as “compounds of the present disclosure”) are shown, for example, above, in the summary of the invention section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present invention may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present invention function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present invention exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

TABLE 1
Compounds of the Present Disclosure

| Compound ID | Structure |
|---|---|
| FSM-206 (i.e., GSK321) | 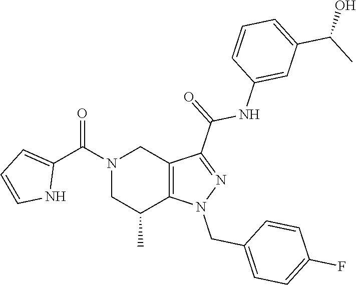 [SMILES: C[C@@H]1CN(C(=O)c2ccc[nH]2)Cc2c(C(=O)Nc3cccc([C@@H](C)O)c3)nn(Cc3ccc(F)cc3)c21] |
| FSM-207 | 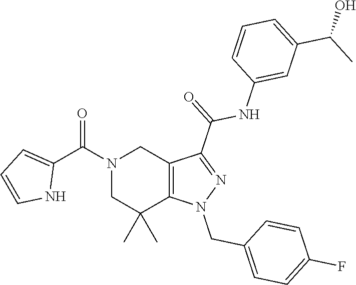 [SMILES: C[C@@H](O)c1cccc(NC(=O)c2nn(Cc3ccc(F)cc3)c3c2CN(C(=O)c2ccc[nH]2)CC3(C)C)c1] |
| FSM-2-302 | 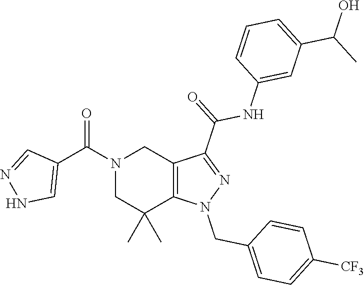 [SMILES: CC(O)c1cccc(NC(=O)c2nn(Cc3ccc(C(F)(F)F)cc3)c3c2CN(C(=O)c2cn[nH]c2)CC3(C)C)c1] |
| FSM-3-02 | 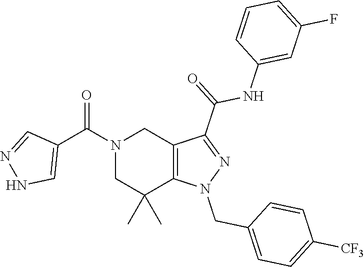 [SMILES: CC1(C)CN(C(=O)c2cn[nH]c2)Cc2c(C(=O)Nc3cccc(F)c3)nn(Cc3ccc(C(F)(F)F)cc3)c21] |
| FSM-2-301 | 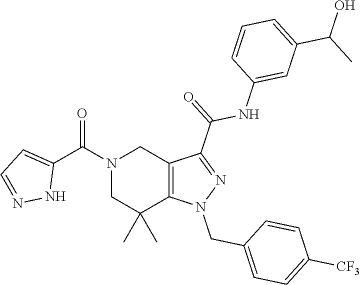 [SMILES: CC(O)c1cccc(NC(=O)c2nn(Cc3ccc(C(F)(F)F)cc3)c3c2CN(C(=O)c2ccn[nH]2)CC3(C)C)c1] |
| FSM-3-01 | 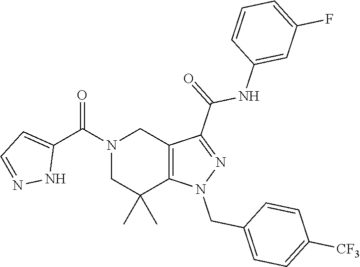 [SMILES: CC1(C)CN(C(=O)c2ccn[nH]2)Cc2c(C(=O)Nc3cccc(F)c3)nn(Cc3ccc(C(F)(F)F)cc3)c21] |
| FSM-2-275 | 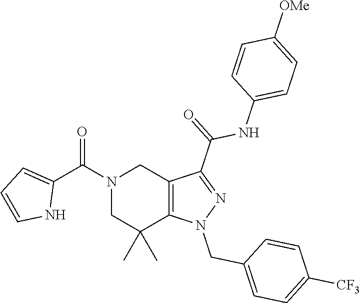 [SMILES: COc1ccc(NC(=O)c2nn(Cc3ccc(C(F)(F)F)cc3)c3c2CN(C(=O)c2ccc[nH]2)CC3(C)C)cc1] |
| FSM-2-276 | 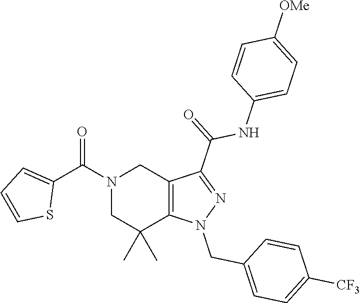 [SMILES: COc1ccc(NC(=O)c2nn(Cc3ccc(C(F)(F)F)cc3)c3c2CN(C(=O)c2cccs2)CC3(C)C)cc1] |
| FSM-2-277 | 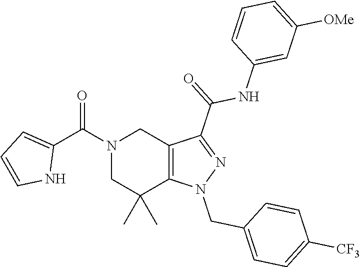 [SMILES: COc1cccc(NC(=O)c2nn(Cc3ccc(C(F)(F)F)cc3)c3c2CN(C(=O)c2ccc[nH]2)CC3(C)C)c1] |
| FSM-2-278 | 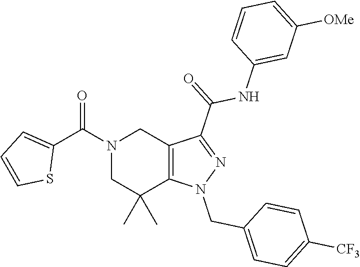 [SMILES: COc1cccc(NC(=O)c2nn(Cc3ccc(C(F)(F)F)cc3)c3c2CN(C(=O)c2cccs2)CC3(C)C)c1] |
| FSM-3-002 | 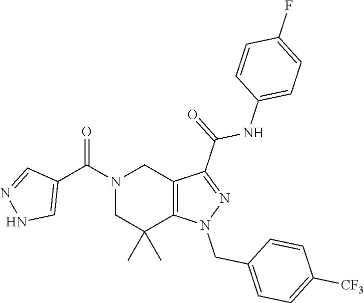 [SMILES: CC1(C)CN(C(=O)c2cn[nH]c2)Cc2c(C(=O)Nc3ccc(F)cc3)nn(Cc3ccc(C(F)(F)F)cc3)c21] |
| AC-2-069 (i.e., AC-1-277) | 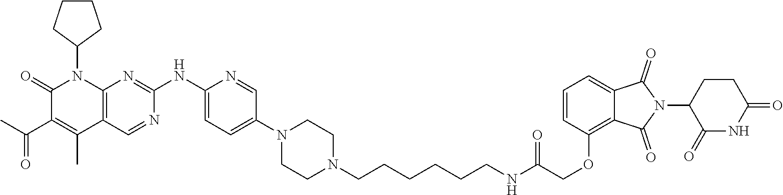 [SMILES: CC(=O)c1c(C)c2cnc(Nc3ccc(N4CCN(CCCCCCNC(=O)COc5cccc6c5C(=O)N(C5CCC(=O)NC5=O)C6=O)CC4)cn3)nc2n(C2CCCC2)c1=O] |
| FSM-3-17 | 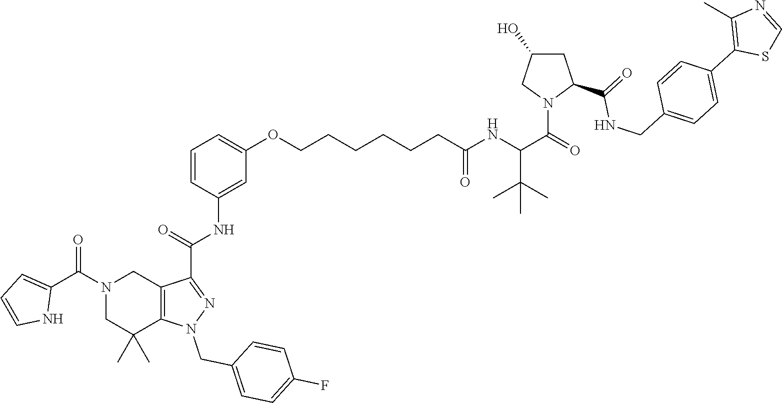 [SMILES: Cc1ncsc1-c1ccc(CNC(=O)[C@@H]2C[C@@H](O)CN2C(=O)C(NC(=O)CCCCCCOc2cccc(NC(=O)c3nn(Cc4ccc(F)cc4)c4c3CN(C(=O)c3ccc[nH]3)CC4(C)C)c2)C(C)(C)C)cc1] |
| FSM-2-95 | 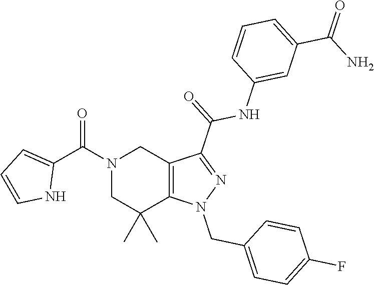 [SMILES: CC1(C)CN(C(=O)c2ccc[nH]2)Cc2c(C(=O)Nc3cccc(C(N)=O)c3)nn(Cc3ccc(F)cc3)c21] |
| FSM-2-96 | 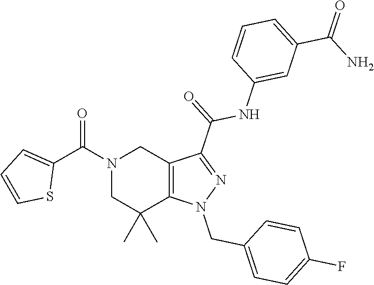 [SMILES: CC1(C)CN(C(=O)c2cccs2)Cc2c(C(=O)Nc3cccc(C(N)=O)c3)nn(Cc3ccc(F)cc3)c21] |
| FSM-2-97 | 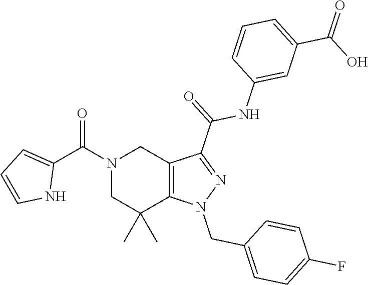 [SMILES: CC1(C)CN(C(=O)c2ccc[nH]2)Cc2c(C(=O)Nc3cccc(C(=O)O)c3)nn(Cc3ccc(F)cc3)c21] |
| FSM-2-98 | 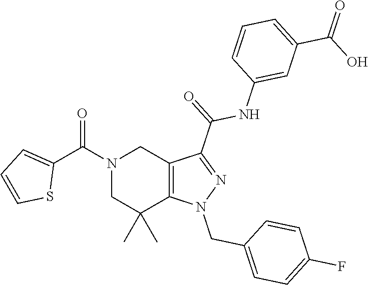 [SMILES: CC1(C)CN(C(=O)c2cccs2)Cc2c(C(=O)Nc3cccc(C(=O)O)c3)nn(Cc3ccc(F)cc3)c21] |
| FSM-2-113 | 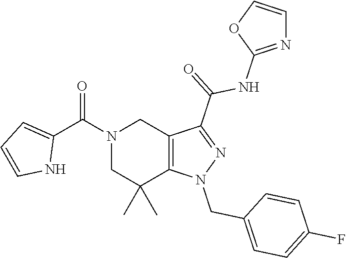 [SMILES: CC1(C)CN(C(=O)c2ccc[nH]2)Cc2c(C(=O)Nc3ncco3)nn(Cc3ccc(F)cc3)c21] |
| FSM-2-114 | 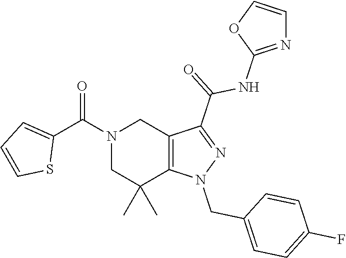 [SMILES: CC1(C)CN(C(=O)c2cccs2)Cc2c(C(=O)Nc3ncco3)nn(Cc3ccc(F)cc3)c21] |
| FSM-2-115 | 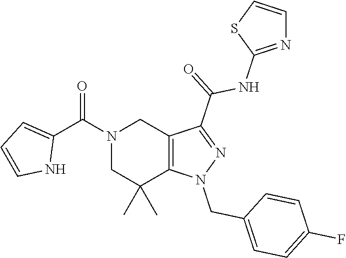 [SMILES: CC1(C)CN(C(=O)c2ccc[nH]2)Cc2c(C(=O)Nc3nccs3)nn(Cc3ccc(F)cc3)c21] |
| FSM-2-116 | 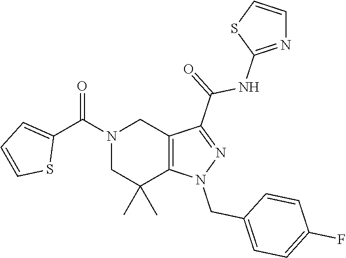 [SMILES: CC1(C)CN(C(=O)c2cccs2)Cc2c(C(=O)Nc3nccs3)nn(Cc3ccc(F)cc3)c21] |
| FSM-5-141 | 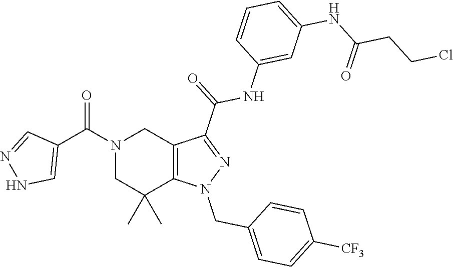 [SMILES: CC1(C)CN(C(=O)c2cn[nH]c2)Cc2c(C(=O)Nc3cccc(NC(=O)CCCl)c3)nn(Cc3ccc(C(F)(F)F)cc3)c21] |
| FSM-5-157 | 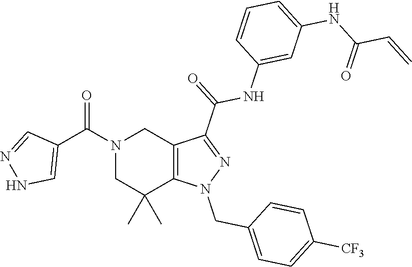 [SMILES: C=CC(=O)Nc1cccc(NC(=O)c2nn(Cc3ccc(C(F)(F)F)cc3)c3c2CN(C(=O)c2cn[nH]c2)CC3(C)C)c1] |
| IAP-1-23 | 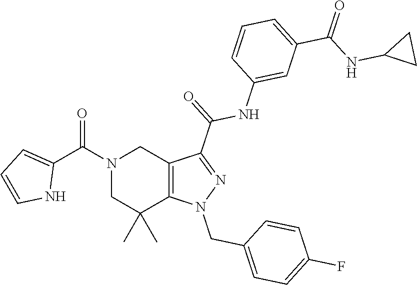 [SMILES: CC1(C)CN(C(=O)c2ccc[nH]2)Cc2c(C(=O)Nc3cccc(C(=O)NC4CC4)c3)nn(Cc3ccc(F)cc3)c21] |
| IAP-1-34 | 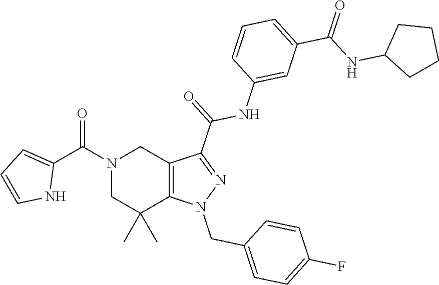 [SMILES: CC1(C)CN(C(=O)c2ccc[nH]2)Cc2c(C(=O)Nc3cccc(C(=O)NC4CCCC4)c3)nn(Cc3ccc(F)cc3)c21] |
| IAP-1-35 | 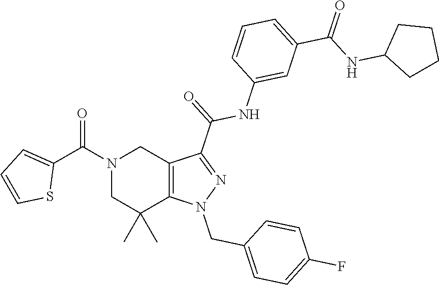 [SMILES: CC1(C)CN(C(=O)c2cccs2)Cc2c(C(=O)Nc3cccc(C(=O)NC4CCCC4)c3)nn(Cc3ccc(F)cc3)c21] |
| IAP-1-37 | 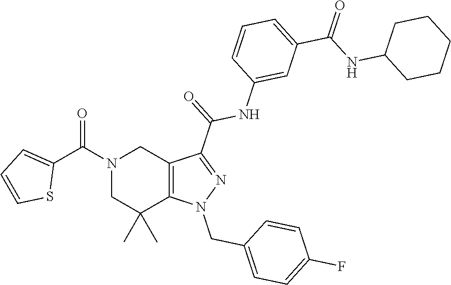 [SMILES: CC1(C)CN(C(=O)c2cccs2)Cc2c(C(=O)Nc3cccc(C(=O)NC4CCCCC4)c3)nn(Cc3ccc(F)cc3)c21] |
| IAP-1-39 | 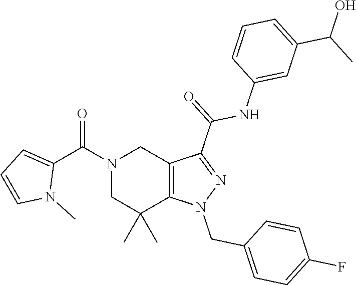 [SMILES: CC(O)c1cccc(NC(=O)c2nn(Cc3ccc(F)cc3)c3c2CN(C(=O)c2cccn2C)CC3(C)C)c1] |
| IAP-1-40 | 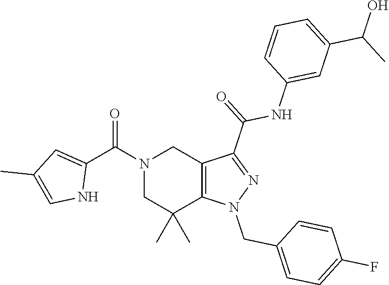 [SMILES: Cc1c[nH]c(C(=O)N2Cc3c(C(=O)Nc4cccc(C(C)O)c4)nn(Cc4ccc(F)cc4)c3C(C)(C)C2)c1] |
| AP-09-271 | 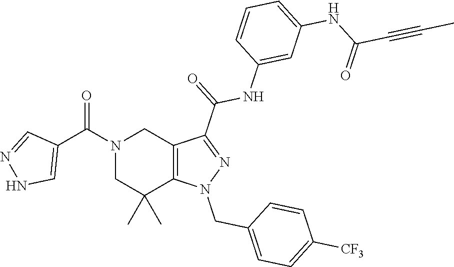 [SMILES: CC#CC(=O)Nc1cccc(NC(=O)c2nn(Cc3ccc(C(F)(F)F)cc3)c3c2CN(C(=O)c2cn[nH]c2)CC3(C)C)c1] |
| VV-1-19 | 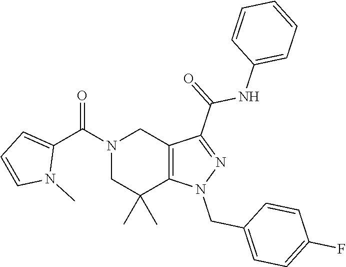 [SMILES: Cn1cccc1C(=O)N1Cc2c(C(=O)Nc3ccccc3)nn(Cc3ccc(F)cc3)c2C(C)(C)C1] |

### IV. Pharmaceutical Compositions and Methods of Treatment

### A. Pharmaceutical Compositions and Routes of Administration

The exact formulation, route of administration and dosage for the compositions disclosed herein can be chosen by an individual physician or clinician in view of a patient's condition (see e.g., Fingl et al., in The Pharmacological Basis of Therapeutics, 1975, Ch. 1). It should be noted that the attending physician would know how to and when to terminate, interrupt, or adjust administration due to toxicity, or to organ dysfunctions, etc. Conversely, the attending physician would also know to adjust treatment to higher levels if the clinical response were not adequate (in light of or precluding toxicity aspects). The magnitude of an administered dose in the management of the disorder of interest can vary with the severity of the condition to be treated and to the route of administration. The severity of the condition may, for example, be evaluated, in part, by standard prognostic evaluation methods. Further, the dose and perhaps dose frequency, can also vary according to circumstances, e.g., the age, body weight, and response of the individual patient. A program comparable to that discussed above also may be used in veterinary medicine.

Depending on the specific cancer being treated and the targeting method selected, such agents may be formulated and administered systemically or locally. Techniques for formulation and administration may be found in the art.

The compounds can be administered to a patient in combination with a pharmaceutically acceptable carrier, diluent, or excipient. The phrase “pharmaceutically acceptable” refers to those ligands, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” includes any and all solvents, dispersion media, diluents, coatings, surfactants, antioxidants, preservatives (e.g., antibacterial agents, antifungal agents), isotonic agents, absorption delaying agents, salts, buffers, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the chemotherapeutic or pharmaceutical compositions is contemplated.

An IDH1 inhibitor may be combined with different types of carriers depending on whether it is to be administered in solid, liquid or aerosol form, and whether it need to be sterile for such routes of administration as injection. The present disclosure can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, injection, infusion, continuous infusion, localized perfusion bathing target cells directly, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

When administered to a subject, effective amounts will depend, of course, on the particular cancer being treated; the genotype of the specific cancer; the severity of the cancer; individual patient parameters including age, physical condition, size and weight, concurrent treatment, frequency of treatment, and the mode of administration. These factors are well known to the physician and can be addressed with no more than routine experimentation. In some embodiments, it is preferred to use the highest safe dose according to sound medical judgment.

In certain embodiments, pharmaceutical compositions may comprise, for example, at least about 0.1% of an isocitrate dehydrogenase 1 inhibitor. In other embodiments, an isocitrate dehydrogenase 1 inhibitor may comprise between about 2% to about 75% of the weight of the unit, or between about 25% to about 60%, for example, and any range derivable therein. In other non-limiting examples, a dose may also comprise from about 0.1 mg/kg/body weight, 0.5 mg/kg/body weight, 1 mg/kg/body weight, about 5 mg/kg/body weight, about 10 mg/kg/body weight, about 20 mg/kg/body weight, about 30 mg/kg/body weight, about 40 mg/kg/body weight, about 50 mg/kg/body weight, about 75 mg/kg/body weight, about 100 mg/kg/body weight, about 200 mg/kg/body weight, about 350 mg/kg/body weight, about 500 mg/kg/body weight, about 750 mg/kg/body weight, to about 1000 mg/kg/body weight or more per administration, and any range derivable therein. In non-limiting examples of a derivable range from the numbers listed herein, a range of about 10 mg/kg/body weight to about 100 mg/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof.

The isocitrate dehydrogenase 1 inhibitors as described herein may be formulated into a composition in a free base, neutral or salt form. Pharmaceutically acceptable salts include the salts formed with the free carboxyl groups derived from inorganic bases such as for example, sodium, potassium, ammonium, calcium or ferric hydroxides; or such organic bases as isopropylamine, triethylamine, histidine or procaine.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are optionally provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose (HPC); or combinations thereof such methods. In many cases, it will be preferable to include isotonic agents, such as, for example, sugars, sodium chloride or combinations thereof.

Sterile injectable solutions are prepared by incorporating the active compounds in the required amount of the appropriate solvent with various other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose.

The composition should be stable under the conditions of manufacture and storage, and preserved against the contaminating action of microorganisms, such as bacteria and fungi. Thus, preferred compositions have a pH greater than about 5, preferably from about 5 to about 8, more preferably from about 5 to about 7. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin or combinations thereof.

### B. Combined Therapy

In the context of the present disclosure, it also is contemplated the IDH1 inhibitor could be used in conjunction with chemo- or radiotherapeutic intervention, or other treatments. It also may prove effective, in particular, to combine the IDH1 inhibitor with other therapies that target different aspects of cancer cell function.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with the IDH1 inhibitor and at least one other agent. These compositions would be provided in a sequential or combined amount effective to kill or inhibit proliferation of the cell. This process may involve contacting the cells with the IDH1 inhibitor and the other agent(s) or factor(s) at the same time. This may be achieved by contacting the cell with a single composition or pharmacological formulation that includes both agents, or by contacting the cell with two distinct compositions or formulations, at the same time, wherein one composition includes the interferon prodrugs according to the present disclosure and the other includes the other agent.

Alternatively, the IDH1 inhibitor therapy may precede or follow the other agent treatment by intervals ranging from minutes to weeks. In embodiments where the other agent and the interferon prodrugs are applied separately to the cell, one would generally ensure that a significant period of time did not expire between each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either interferon prodrugs or the other agent will be desired. Various combinations may be employed, where the IDH1 inhibitor therapy is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve cell killing, both agents are delivered to a cell in a combined amount effective to kill the cell.

Administration of the IDH1 inhibitors of the present disclosure to a patient will follow general protocols for the administration of chemotherapeutics, taking into account the toxicity, if any. It is expected that the treatment cycles would be repeated as necessary. It also is contemplated that various standard therapies or adjunct cancer therapies, as well as surgical intervention, may be applied in combination with the described active agent(s). These therapies include but are not limited to chemotherapy, radiotherapy, immunotherapy, gene therapy and surgery.

### 1. Chemotherapy

Cancer therapies can also include a variety of combination therapies with both chemical and radiation-based treatments. Combination chemotherapies include the use of chemotherapeutic agents such as, cisplatin, etoposide, irinotecan, camptostar, topotecan, paclitaxel, docetaxel, epothilones, taxotere, tamoxifen, 5-fluorouracil, methoxtrexate, temozolomide, cyclophosphamide, SCH 66336, R115777, L778,123, BMS 214662, IRESSA™ (gefitinib), TARCEVA™ (erlotinib hydrochloride), antibodies to EGFR, GLEEVEC™ (imatinib), intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, pentostatine, vinblastine, vincristine, vindesine, bleomycin, doxorubicin, dactinomycin, daunorubicin, epirubicin, idarubicin, mithramycin, deoxycoformycin, Mitomycin-C, L-Asparaginase, teniposide, 17α-Ethinylestradiol, Diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesterone acetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, Avastin, herceptin, Bexxar, Velcade, Zevalin, Trisenox, Xeloda, Vinorelbine, Porfimer, Erbitux™ (cetuximab), Liposomal, Thiotepa, Altretamine, Melphalan, Trastuzumab, Lerozole, Fulvestrant, Exemestane, Fulvestrant, Ifosfomide, Rituximab, C225, Campath, carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, paclitaxel, gemcitabine, navelbine, farnesyl-protein transferase inhibitors, transplatinum, 5-fluorouracil, vincristine, vinblastine and methotrexate, or any analog or derivative variant of the foregoing.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (e.g., 3 to 4 wks), to single doses of 2000 to 6000 roentgens. Dosage ranges for radioisotopes vary widely, and depend on the half-life of the isotope, the strength and type of radiation emitted, and the uptake by the neoplastic cells. The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### 3. Immunotherapy

In the context of cancer treatment, immunotherapeutics, generally, rely on the use of immune effector cells and molecules to target and destroy cancer cells. Trastuzumab (Herceptin®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, —IFN, chemokines such as MIP-1, MCP-1, IL-8, CCL18 and growth factors such as FLT3 ligand. Combining immune stimulating molecules, either as proteins or using gene delivery in combination with a tumor suppressor has been shown to enhance anti-tumor effects. Moreover, antibodies against any of these compounds can be used to target the anti-cancer agents discussed herein.

Examples of immunotherapies currently under investigation or in use are immune adjuvants, e.g., Mycobacterium bovis, Plasmodium falciparum, dinitrochlorobenzene and aromatic compounds, cytokine therapy, e.g., interferons α and β, IL-1, IL-2, GM-CSF and TNF, and monoclonal antibodies, e.g., anti-ganglioside GM2, anti-HER-2, anti-p185. It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant. In adoptive immunotherapy, the patient's circulating lymphocytes, or tumor infiltrated lymphocytes, are isolated in vitro, activated by lymphokines such as IL-2 or transduced with genes for tumor necrosis, and readministered.

Two emerging immunotherapies are immune checkpoint blockage therapy and adoptive T cell therapy, have resulted in promising results in certain types of cancer patients, but the overall effective rates are still varied among the tumor types. One of the key determinants for the therapeutic efficacy and immune responses is functional state of the transferred/preexisting T cells in the tumor suppressive microenvironment. It is now well-recognized that T cells are exhausted with expression of inhibitory receptors in the tumor microenvironment in cancer patients, which is also accompanied with the loss of effector functions and proliferation. However, the current checkpoint blockage therapy using antibodies to target PD1/PDL1 or/and CTLA4 only have limited success rates from 15% to 35%, suggesting that combining these therapies with other cancer therapies may be advantageous.

### 4. Gene Therapy

In yet another embodiment, the secondary treatment is a secondary gene therapy in which a therapeutic polynucleotide is administered before, after, or at the same time a first chemotherapeutic agent. Delivery of the chemotherapeutic agent in conjunction with a vector encoding a gene product will have a combined anti-hyperproliferative effect on target tissues.

### 5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue. The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### V. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof, in a divalent context “phosphate” means —OP(O)(OH)O— or a deprotonated form thereof, “mercapto” means —SH; and “thin” means ═S; “thiocarbonyl” means —C(═S)—; “sulfonyl” means —S(O)₂⁻; and “sulfinyl” means —S(O)—.

In the context of chemical formulas, the symbol “—” means a single bond, “═” means a double bond, and “—” means triple bond. The symbol “ [symbol] ” represents an optional bond, which if present is either single or double. The symbol “ [symbol] ” represents a single bond or a double bond. Thus, the formula

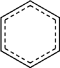

covers, for example,

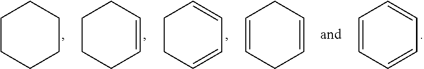

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g., [symbol] for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

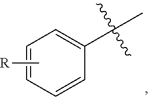

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

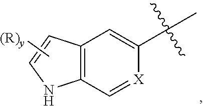

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C═n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl(C≤8)”, “heteroaryl_{(C≤8)}”, and acyl_{(C≤8)} is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic it system. An aromatic compound or chemical group may be depicted as a single resonance structure; however, depiction of one resonance structure is taken to also refer to any other resonance structure. For example:

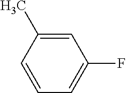

is also taken to refer to

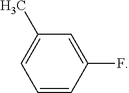

Aromatic compounds may also be depicted using a circle to represent the delocalized nature of the electrons in the fully conjugated cyclic 71 system, two non-limiting examples of which are shown below:

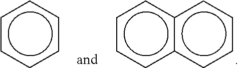

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, =CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

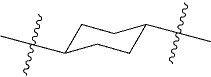 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. The term “alkynediyl” refers to a divalent unsaturated aliphatic group, with two carbon atoms as points of attachment, a linear or branched acyclic structure, at least one nonaromatic carbon-carbon triple bond, no carbon-carbon double bonds, and no atoms other than carbon and hydrogen. The groups —C≡C—, —C≡CCH₂—, and —CH₂C═CCH₂— are non-limiting examples of alkynediyl groups. It is noted that while the alkynediyl group is aliphatic, once connected at both ends, this group is not precluded from forming part of an aromatic structure. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

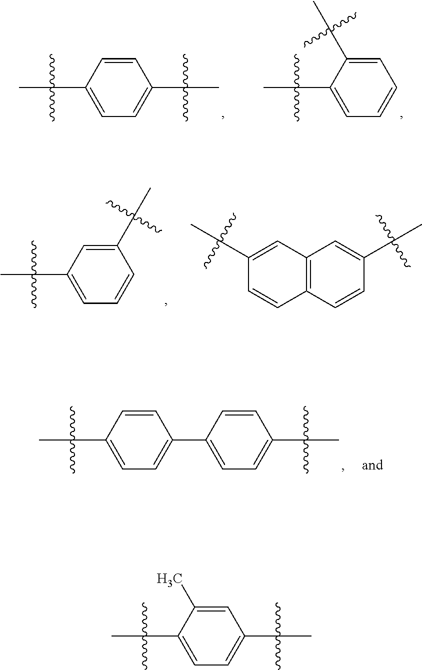

An “arene” refers to the class of compounds having the formula H—R, wherein R is aryl as that term is defined above. Benzene and toluene are non-limiting examples of arenes.

The term “aralkyl” refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The terms “dicycloalkylamino”, “dialkenylamino”, “dialkynylamino”, “diarylamino”, “diaralkylamino”, “diheteroaryl amino”, “diheterocycloalkylamino”, and “dialkoxyamino”, refers to groups, defined as —NRR′, in which R and R′ are both cycloalkyl, alkenyl, alkynyl, aryl, aralkyl, heteroaryl, heterocycloalkyl, and alkoxy, respectively. Similarly, the term alkyl(cycloalkyl)amino refers to a group defined as —NRR′, in which R is alkyl and R′ is cycloalkyl. The term “amido” (acylamino), when used without the “substituted” modifier, refers to the group —NHR, in which R is acyl, as that term is defined above. A non-limiting example of an amido group is —NHC(O)CH₃.

An “amine protecting group” or “amino protecting group” is well understood in the art. An amine protecting group is a group which prevents the reactivity of the amine group during a reaction which modifies some other portion of the molecule and can be easily removed to generate the desired amine. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxy-benzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxy-benzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH— or PG_{DA}N- wherein PG_{MA} is a monovalent amine protecting group, which may also be described as a “monovalently protected amino group” and PG_{DA} is a divalent amine protecting group as described above, which may also be described as a “divalently protected amino group”.

When a chemical group is used with the “substituted” modifier, one or more hydrogen atom has been replaced, independently at each instance, by —OH, —F, —C₁, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂C₁, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C₁. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “ubiquitin ligase ligand” refers to a chemical group capable of binding ubiquitin ligase. Ubiquitin ligase (also called an E3 ubiquitin ligase or simply an E3 ligase) is a protein that recruits an E2 ubiquitin-conjugating enzyme that has been loaded with ubiquitin, recognizes a protein substrate, and assists or directly catalyzes the transfer of ubiquitin from the E2 to the protein substrate. The ubiquitin is attached to a lysine on the target protein by an isopeptide bond. E3 ligases interact with both the target protein and the E2 enzyme, and so impart substrate specificity to the E2. Commonly, E3s polyubiquitinate their substrate with Lys48-linked chains of ubiquitin, targeting the substrate for destruction by the proteasome. However, one of skill in the art recognizes that many other types of linkages are possible and that each may alter a protein's activity, interactions, or localization. Ubiquitination by E3 ligases regulates diverse areas such as cell trafficking, DNA repair, and signaling and is of profound importance in cell biology. E3 ligases are also key players in cell cycle control, mediating the degradation of cyclins, as well as cyclin dependent kinase inhibitor proteins. The human genome encodes over 600 putative E3 ligases, allowing for tremendous diversity in substrates. Non-limiting examples of ubiquitin ligase ligands include the von Hippel-Lindau (VHL) ligand, a cIAP1 ligand, a MDM2 ligand, a CRBN ligand, a CUL2 ligand, or other ligand which binds to one or more of the proteins of the ubiquitin protein complex especially the E3 component of this complex.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### II. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Biological Data

| | Microsomal Stability |
|---|---|
| | (Mouse, [Compound] = 0.5 μM, n = 1) |
| Control | Midazolam |
| Test compound | Half-life (t_{1/2}) (min) |
| FSM-2-302 | 56.6 |
| FSM-3-17 | 19.5 |
| FSM-3-002 | 147 |
| Midazolam | 1.76 |

| | Compound | | | | | | |
|---|---|---|---|---|---|---|---|
| | FSM-2- | FSM-3- | FSM- | FSM-2- | FSM-2- | FSM-2- | FSM-2- |
| | 302 | 02 | 206 | 275 | 276 | 277 | 278 |
| Fit | #Ok | #Ok | #Ok | #Ok | #Ok | #Ok | #Ok |
| Bottom | 4.0 | −0.9 | −0.8 | 3.6 | 15.8 | 11.8 | 4.6 |
| Top | 97.9 | 95.0 | 98.0 | 99.1 | 98.9 | 99.3 | 98.0 |
| IC₅₀, nM | 36 | 113 | 22 | 3.3 | 2.8 | 2.9 | 2.9 |
| Slope | 1.0 | 1.0 | 1.0 | 1.3 | 1.4 | 1.0 | 1.0 |
| Chart | #Ok | #Ok | #Ok | #Ok | #Ok | #Ok | #Ok |
| Comments | | | | | | | |

| S. No | Compound ID | M/W | Mass (mg) |
|---|---|---|---|
| 1 | AC-2-069 | 860.97 | 1.51 |
| | (AC-1-277) | | |
| 2 | FSM-2-302 | 566.23 | 1.20 |
| 3 | FSM-3-17 | 1027.48 | 1.20 |
| 4 | FSM-3-002 | 540.19 | 1.35 |
| S. No | Compound ID | M/W | Mass (mg) |
| 1 | FSM-2-95 | 514.51 | 1.20 |
| 2 | FSM-2-96 | 531.61 | 2.90 |
| 3 | FSM-2-97 | 515.55 | 1.01 |
| 4 | FSM-2-98 | 532.59 | 1.50 |
| 5 | FSM-2-113 | 462.49 | 0.46 |
| 6 | FSM-2-114 | 479.53 | 1.47 |
| 7 | FSM-2-115 | 478.55 | 2.36 |
| 8 | FSM-2-116 | 495.59 | 1.09 |
| 9 | IAP-1-23 | 554.63 | 1.20 |
| 10 | IAP-1-34 | 582.68 | 2.50 |
| 11 | IAP-1-35 | 599.73 | 2.10 |
| 12 | IAP-1-37 | 613.75 | 2.46 |
| 13 | IAP-1-39 | 529.62 | 2.60 |
| 14 | IAP-1-40 | 529.62 | 2.30 |
| 15 | VV-1-19 | 485.56 | 2.06 |

| Compound | IC₅₀, Trial 1 | IC₅₀, Trial 2 | Mean IC₅₀ |
|---|---|---|---|
| FSM-206 | 25 | 27 | 26 |
| FSM-207 | 38 | 43 | 40 |
| FSM-103 | 608 | 695 | 650 |
| FSM-203 | 102 | 106 | 104 |
| FSM-104 | 255 | 264 | 260 |
| FSM-102 | 495 | 502 | 499 |
| FSM-1-131 | 4675 | 5328 | 4991 |
| FSM-1-132 | 5660 | 5750 | 5705 |
| FSM-1-133 | 2434 | 2584 | 2508 |
| FSM-1-195 | 167 | 178 | 172 |
| FSM-1-196 | 85 | 84 | 85 |
| FSM-1-197 | 175 | 183 | 179 |
| FSM-1-199 | 655 | 658 | 657 |
| FSM-1-266 | 23 | 20 | 22 |
| FSM-1-267 | 21 | 18 | 20 |
| FSM-1-268 | 145 | 137 | 141 |
| FSM-1-287 | 96 | 93 | 95 |
| FSM-1-288 | 38 | 35 | 37 |
| FSM-2-20 | 127 | 135 | 131 |
| FSM-2-21 | 142 | 149 | 146 |
| FSM-2-23 | >10000 | >10000 | >10000 |
| FSM-2-24 | 1940 | 3618 | 2649 |
| FSM-2-30 | 379 | 327 | 352 |
| FSM-2-31 | 207 | 204 | 205 |
| FSM-2-33 | >10000 | >10000 | >10000 |
| FSM-2-34 | >10000 | >10000 | >10000 |
| VV-1-8 | 131 | 127 | 129 |
| VV-1-9 | 1388 | 1470 | 1428 |
| FSM-2-75 | 390 | 408 | 399 |
| FSM-2-68 | 9047 | 9554 | 9297 |
| FSM-2-69 | 4324 | 5151 | 4720 |
| FSM-2-70 | 8929 | 8949 | 8939 |
| FSM-2-53 | >10000 | >10000 | >10000 |
| FSM-2-54 | >10000 | >10000 | >10000 |
| FSM-2-55 | 8473 | 9620 | 9028 |
| FSM-2-65 | 229 | 222 | 225 |
| FSM-2-66 | 74 | 86 | 80 |
| FSM-2-67 | 188 | 209 | 198 |
| VV-1-19 | 73 | 73 | 73 |
| IAP-1-23 | 1031 | 961 | 996 |
| IAP-1-34 | 88 | 95 | 91 |
| IAP-1-35 | 69 | 70 | 69 |
| IAP-1-37 | 19 | 18 | 19 |
| IAP-1-39 | 45 | 47 | 46 |
| IAP-1-40 | 520 | 475 | 497 |
| FSM-2-95 | 309 | 294 | 301 |
| FSM-2-96 | 265 | 253 | 259 |
| FSM-2-97 | >10000 | 7829 | 7829 |
| FSM-2-98 | 6256 | 5066 | 5630 |
| FSM-2-113 | 1272 | 1051 | 1156 |
| FSM-2-114 | 1378 | 1231 | 1302 |
| FSM-2-115 | >10000 | >10000 | >10000 |
| FSM-2-116 | >10000 | 8111 | 8111 |
| BAY-1436032 | 21 | 22 | 21 |
| Vorasidenib | 6.2 | 6.4 | 6.3 |
| IDH-305 | 65 | 64 | 64 |

| | FSM-05-141 | FSM-05-157 |
|---|---|---|
| k6 (off rate for 2nd step), | 0.002 | 0.001 |
| min−1 | | |
| k5 (on rate for 2nd step, | 0.257 | 0.306 |
| min−1 | | |
| KiAPP (affinity for 1st | 12456 | 23591 |
| step), nM | | |
| KiAPP* (affinity for 2nd | 82.2 | 64.8 |
| step), nM | | |

Note:
The k6 values are very close to 0, especially for 157, which is consistent with irreversible binding.

### Example 2—Materials and Methods

Cell lines, cultures, and reagents. All cell lines were obtained from ATCC, except murine PC cells (KPC K8484: Kras^{G12D/+};Trp53^{R172H/+}; Pdx1-Cre). KPC cells were provided by the Darren Carpizo lab. Mycoplasma screening was performed using a MycoAlert detection kit (Lonza). Cell lines were maintained at 37° C. and 5% CO₂. For standard cell culture, cells were grown in DMEM containing 25 mM glucose and 4 mM glutamine, and supplemented with 10% FBS, 1% penicillin/streptomycin, and prophylactic doses of plasmocin (Life Technologies, MPP-01-03) to prevent mycoplasma infection. Glucose withdrawal was performed to simulate low glucose conditions in the PC microenvironment. For low-glucose experiments, glucose-free DMEM (Life Technologies, 21013-024) was supplemented with 2.5 mM glucose, 10% FBS, and penicillin-streptomycin. For experiments with low magnesium and glucose, magnesium sulphate-depleted DMEM (Cell Culture Technologies, 964DME-0619) was supplemented with the indicated concentrations of MgSO₄ and glucose. Standard or high Mg²⁺ refers to levels that approximate serum (0.8 to 1.5 mM); low levels approximate the tumor microenvironment and are generally below 0.4 mM. For rescue experiments, Sodium citrate dihydrate (BP327, Fisher Scientific), GSH (G6013, Sigma), αKG (Sigma-Aldrich, 75890), and NAC (N-Acetyl-L-cysteine, Sigma, A9165) were used.

CRISPR/Cas9 editing of IDH1 in pancreatic cancer cells. IDH1 knockout was performed using a gRNA: GTAGATCCAATTCCACGTAGGG (Sigma, target ID, HS0000323225 (SEQ ID NO: 1)). A negative control plasmid (CRISPR06-1EA) was used in isogenic cells. Plasmid transfections were performed with lipofectamine 2000 (Life technology, 11668-027). After 48 h, EGFP-expressing cells were sorted by flow cytometry. Clones from parental cell lines (MiaPaCa-2 and Hs766T) were expanded, and genomic DNA and protein were extracted for verification of IDH1 deletion. Herein, cells with IDH1 deletion and isogenic controls are referred to as IDH1−/− and IDH1+/+, respectively.

Cell viability assays. Cell viability was estimated through cell counting using Trypan blue reagent (Life Technologies, 15250061) or by DNA quantitation via PicoGreen dsDNA assay (Life Technologies, P7589).

Clonogenic assay. 1000-2000 cells per well were plated in six-well plates. Media was not changed during experiments unless indicated. For AG-120 experiments, cells were first cultured with indicated MgSO₄ media for 24 hours, followed by AG-120 treatment under 4 mM glutamine, 5% FBS, and indicated glucose levels. Upon completion of the experiments, colonies were fixed in a reagent containing 80% methanol and stained with 0.5% crystal violet. To determine relative growth, dye was extracted from stained colonies with 10% acetic acid and the associated absorbance measured at 600 nm using a Microplate Reader (GloMax Explorer system, Promega).

ROS and 8-OHdG quantification. Cells were incubated in a 96-well plate with 10 μM H₂-DCFDA (Life Technology, D399) for 45 min in serum-free media to detect total intracellular ROS. For mitochondrial-specific ROS, cells were incubated with 5 μM MitoSOX Red (Life Technologies, M36008) for 30 min in serum-free media. Cells were washed with PBS, and fluorescence measured according to manufacturer instructions using a Microplate Reader (GloMax Explorer system, Promega). 8-OHdG was measured according to the manufacturer's instructions (Abcam, AB201734). Readouts were normalized to cell number.

Quantitative RT-PCR. Total RNA was extracted using PureLink RNA isolation (Life Technologies, 12183025) and treated with DNase (Life Technologies, AM2222). cDNA was synthesized using 1 μg of total RNA, oligo-dT and MMLV HP reverse transcriptase (Applied Biosystems, 4387406). All PCR reactions were performed in triplicate, and primer sequences are provided in the supplementary section. RT-qPCR acquisition was captured using Bio-Rad CFX96 and analyzed using Bio-Rad CFX Manager 2.0 software.

RNA-sequencing and analyses. RNA quality was assessed via the Agilent 2100 Bioanalyzer (Agilent Technologies). Strand-specific RNA-seq library was prepared using NEBNext Ultra II Directional RNA Library Prep Kit (NEB, Ipswich, MA) according to the manufacturer's protocols. RNA-sequencing was performed using 150-bp paired-end format on a NovaSeq 6000 (Illumina) sequencer. FastQC was used to assess RNA-seq quality and TrimGalore was used for adapter and quality trimming. RNA-seq reads were mapped against hg38 using STAR (v 2.7.0e) aligner with default parameters. DESeq2 analysis generated a list of differentially expressed genes, using an FDR value <0.05. Gene set enrichment analysis was performed using GSEA (v 4.1.0) with Hallmark gene sets (v 7.2).

DNA sequencing. DNA was isolated using the DNeasy blood and tissue kit (Qiagen) according to the manufacturer's protocol. To assess for wild-type IDH1 sequence, a portion of the IDH1 gene exon 4 containing the Arg132 was amplified using either of two pairs of primers (for human IDH1, F: 5′-ACCAAATGGCACCATACGA-3′ (SEQ ID NO: 2); R: 5′-TTCATACCTTGCTTAATGGGTGT-3′ (SEQ ID NO: 3); for mouse IDH1, F: 5′-ATTCTGGGTGGCACTGTCTT-3′ (SEQ ID NO: 4); R: 5′-CTCTCTTAAGGGTGTAGATGCC-3′ (SEQ ID NO: 5)), and sequenced using one of the amplification primers. Gene segments corresponding to the targeted regions were amplified to validate knockout efficacy (F: 5′-GAGGGCTAGCTCAGAAAC-3′ (SEQ ID NO: 6), R: 5′-CATTGTACTATTCTTAGCCACTG-3′ (SEQ ID NO: 7)). Sanger sequencing was performed using the following primer: 5′-TGGCGGTTCTGTGGTAGAG-3′(SEQ ID NO: 8). PCR reactions were generally carried out as follows: 95° C. for 30 sec, 60° C. for 30 sec, and 72° C. for 40 sec for a total of 40 cycles.

Small RNA interference. For siRNA transfections, oligos were obtained from Life technology and transfections were performed using Lipofectamine 2000. siRNA Gene knockdown validation was determined 72 hours after siRNA transfections via qPCR.

Western blot analysis. Total protein was extracted with RIPA buffer (Pierce, 89900) supplemented with protease inhibitor (Life Technologies, 1861280) and quantified using the BCA Protein Assay (Thermo Scientific). Proteins were separated on 4-12% Bis-Tris gels (Life Technologies, MW04125) and transferred to PVDF membranes. Membranes were probed with antibodies against IDH1 (Invitrogen, GT1521) overnight at 4° C. and alpha-Tubulin (Invitrogen, 11224-1-AP) for 1-2 hours at room temperature. Blots were probed with secondary antibodies customized for the Odyssey Imaging system (32106, Thermo Fisher Scientific).

IDH activity assay. Cell-based wtIDH activity is measured by quantitation of IDH-dependent NADPH synthesis (Abcam, ab 102528). The reaction measures the activity of both IDH1 and IDH2 isoenzymes. IDH1 activity is specifically measured using the same assay after transient siRNA silencing of IDH2 (Extended Data FIG. 6i). The opposite approach is used to estimate IDH2 activity. All readouts were normalized to cell number. Cell-free wtIDH1 activity was performed by a fluorometric assay measuring NADPH levels at 355/460 nm. The assay contained 0.25 nM human recombinant IDH1 (RD systems), 30 μM isocitrate, and 30 μM NADP⁺ in 20 μL of 100 mM Tris-HCL, pH8, 0.2 mM DTT, 0.05% CHAPS, and MgCl₂ at the indicated levels. Formation of NADPH was measured over 40 min at 5 min intervals.

IDH binding assay. IDH1 binding was assessed using HTRF technology with 2.5 nM N-terminal HIS tagged IDH1 (ActiveMotif) prebound to an anti-HIS terbium conjugated antibody (PerkinElmer) and 200 nM FITC labeled IDH1 probe, IK-1-012, in a final volume of 10 μL of IDH1 assay buffer (100 mM Tris-HCL, pH8, 0.2 mM DTT, 0.05% CHAPS, and MgCl₂ at the indicated levels). After an incubation time of 60 min at room temperature, the time-resolved fluorescence at excitation of 340 nm and emission at 520 nm and 620 nm was measured using a ClarioStar plate reader (BMG Lab Tech) and the HTRF ratio at 520/620 emission values was calculated. Data are expressed as % inhibition, where 0% is equal to the HTRF ratio in the presence of HIS-IDH11, and 100% is equal to the HTRF ratio in the absence of HIS-IDH1

Cellular Thermal Shift assay. Thermal shift experiments were performed as previously described (72). Briefly, cells were cultured in media containing the indicated experimental concentrations of MgSO₄ under standard conditions (25 mM glucose, 4 mM glutamine and 10% FBS) for 24 hours. Then, cells were treated with either vehicle (DMSO) or AG-120 (1 μM) for 6-8 hours under the indicated Mg²⁺ levels. Cells were then trypsinized, washed with PBS, suspended in PBS (containing protease inhibitors), lysed through three freeze-thaw cycles at liquid nitrogen−37° C., and then heated at the indicated temperatures for three minutes. Preparations were spun down at 13,000 rpm for 10 min at 4° C. to remove denatured proteins, and supernatants were loaded on a Western blot gel. Western blots were performed as described.

Cell bioenergetic assays. Oxygen consumption rates (OCR) and extracellular acidification rates (ECAR) were measured using the Seahorse XFp Extracellular Flux Analyzer (Seahorse Bioscience) according to the manufacturer's instructions. For OCR experiments, cells were cultured in 4 mM glutamine, 5% FBS, and the indicated glucose levels for 24-36 hours. Mitochondrial membrane potential was measured with TMRE staining (Invitrogen, T669) according to the manufacturer's instructions. For AG-120 experiments, cells were treated with AG-120 (2 μM) under 4 mM glutamine, 5% FBS, and indicated glucose levels. NADPH (abcam, ab65349), GSH/GSSG ratio (Promega, V6611), and ATP (abcam, 83355) measurements were performed according to the manufacturer's instructions. All readouts were normalized to cell number.

Immunohistochemistry staining. Samples were prepared with formalin and embedded in paraffin followed by Ki-67 (Cell Signaling, #9027S, rabbit monoclonal) and Cleaved Caspase-3 (Asp175) (Cell Signaling, #9579S, rabbit monoclonal) staining.

Magnesium measurements in tissues from mice. Once animals were euthanized, tissues were collected, washed with 250 mM sucrose and quickly snap frozen in liquid nitrogen. 50 mg of tissues were homogenized in 10% sucrose via sonication on ice, followed by centrifugation at 12,000 rpm for 10 min at 4° C. Supernatants were removed and examined for free Mg²⁺ content by atomic absorbance spectrophotometry (AAS; Agilent Technologies), adjusting for dilutions. Readouts were normalized according to homogenate volume and protein content.

Metabolic profiling. Experiments were performed in biological triplicates and cells were grown in complete growth media in T25 flasks. For metabolic profiling of IDH1⁻¹⁻ and IDH1^{+/+} cells, processing began when cells reached 50% confluency. To prime cells for low glucose condition, standard culture media was changed to low glucose media (2.5 mM glucose, 4 mM glutamine supplemented with 10% dialyzed FBS) for a 38-hour incubation. Next, for metabolic flux profiling, cells were incubated in the media containing 2.5 mM [U-¹³C]glucose (Cambridge Isotope Laboratories, CLM-1396-1) or 4 mM [U-¹³C]glutamine (Cambridge Isotope Laboratories, CLM-1822-H-0.1) for 10 hours. For rescue experiments, once cells reached a 38-hour incubation under low (2.5 mM) glucose, cells were incubated with either unlabeled αKG (4 mM) or sodium citrate (4 mM) for 10 hours.

After incubation period, cells were placed on ice, washed 3× with cold PBS, and lysed using in ice-cold 80:20 methanol:water. For animal tissues and xenograft tumors, fragments weighing 15-25 mg were homogenized in ice-cold 80:20 methanol:water by sonication at 4° C. Unlabeled γ-hydroxybutyrate (50 μM) was added to homogenized lysate as an internal standard. Cell extracts were centrifuged at 14,000 rpm for 15 min at 4° C. Supernatants were then dried using nitrogen gas. Next, to prepare oxime derivatives, samples were incubated with 30 μl of freshly made MOX solution (40 mg methoxyamine hydrochloride in 1 ml of pyridine) for 45 min at 45° C. This was followed by incubation with 70 μl of MTBSTFA (N-(tert-butyldimethylsilyl)-N-methyltrifluoroacetamide) for 45 min at 45° C. to make silylated derivatives.

For glucose measurements, after sample drying with nitrogen gas, dried lysates were mixed with pyridine:acetic anhydride (1:2) solution and incubated at 60° C. for 30 min to convert glucose to its pentaacetate derivative. Samples were allowed to air dry at room temperature, and then reconstituted in 100 μl of ethyl acetate. Results were normalized to m+6 glucose, as internal standard. GC-MS analyses were performed using a Hewlett Packard 5973 Turbo Pump Mass Selective Detector and a Hewlett Packard 6980 Gas Chromatograph equipped with a DB-5 ms GC Column (60 m×0.25 mm×0.25 um, Agilent Technologies). Samples were injected in splitless mode. The column temperature was initially set at 100° C. and held for one minute, then ramped 8° C./min until 170° C. and held for 5 min. Samples were then ramped 5° C./min until 200° C., and held for 5 min. Finally, samples were ramped 10° C./min until 300° C., and held 10 min. Masses were monitored via the Scan acquisition mode. Metabolomics data were analyzed using the MSD ChemStation Software, version: F.01.03.2357 (Agilent Technologies). Metabolite counts were normalized using cell number from a parallel culture flask plated at an equivalent cell density. Isotopologue abundances were corrected for natural abundance using correction matrix (REF). Metabolite pool sizes were determined using internal standard. Reported absolute metabolic flux was determined as fractional flux multiplied by the pool size and normalized per number of cells and experimental time (reported metabolic rate is expressed as nmols/hour 10⁶ cells).

In vivo studies. All experiments involving mice were approved by the CWRU Institutional Animal Care Regulations and Use Committee (IACUC, protocol 2018-0063). Mice were maintained under pathogen-free conditions in the animal facility. No additional food or nutrient-contained bedding provided to the animals during these studies. Six-to-eight-week-old, female, athymic nude mice (Foxn1 nu/nu) were purchased from Harlan Laboratories (#6903M) through the ARC CWRU. Mice with subcutaneous PDX were purchased from The Jackson Laboratory (#TM01212). KPC (Kras^{G12D/+};Trp53^{R172H/+}; Pdx1-Cre) mice have been described previously (73). Mice on a mixed background were bred in-house at the CRUK Beatson Institute and maintained in conventional caging with environmental enrichment, access to standard chow and water ad libitum. Genotyping was performed by Transnetyx (Cordoba, TN, USA). Mice were monitored 3 times weekly and when a diagnosis of pancreatic cancer was made by abdominal palpation, this was confirmed by high-resolution ultrasound imaging, using the VisualSonics Vevo 3100 preclinical imaging platform (FUJIFILM VisualSonics, Toronto, Canada). Anesthesia was induced and maintained with a mixture of isoflurane and medical air. Mice were imaged weekly to monitor tumor progression and tumor volume was assessed for each mouse and plotted longitudinally. Mice of both sexes were recruited onto study and culled by Schedule 1 method, as per Institutional guidelines, when exhibiting moderate symptoms of PDAC (swollen abdomen, loss of body conditioning resembling cachexia, reduced mobility). All experiments with KPC mice were performed under a UK Home Office license and approved by the University of Glasgow Animal Welfare and Ethical Review Board. Tamoxifen-inducible KP^{lox/lox}C mice were purchased from The Jackson Laboratory (#032429). Tamoxifen dissolved in corn oil at 20 mg/ml, and animals received tamoxifen (75 mg/kg, IP administration, once daily) for six consecutive days. For flank xenograft experiments, 1×10⁶ cells were suspended in 200 μL of a PBS:matrigel solution (1:1) and injected subcutaneously into the right flank of mice. For orthotopic experiments, 4×10⁴ Luciferase-expressing KPC K8484 cells were suspended in 50 μL of a PBS:matrigel solution (1:1) and injected into the pancreas of C57BL/6 mice at 12 weeks of age. Equal number of male and female mice were used. Mice were anesthetized using isoflurane gas. After ensuring appropriate depth of anesthesia, a 0.5 cm left subcostal incision was made to gain access into the peritoneal cavity. The tail of the pancreas was externalized and the mixture was carefully injected into the pancreas. The pancreatic tail was left alone for one minute to limit tumor cell leakage, and then returned to the peritoneal cavity. The incision was then closed with a combination of permanent suture and skin clips. On postoperative day 6-7, the presence of pancreatic tumors was confirmed with bioluminescence imaging, IVIS, using 100 μl intraperitoneal Luciferin injection (50 mg/mL). Mice with confirmed tumors were then randomized to the indicated treatment arms. For the indicated experiments, hyperglycemia was induced by allowing mice to drink high glucose content water (30% dextrose; abbreviated as D30 water) starting two weeks before cancer cell implantation. 3DNA nanocarriers were prepared as described previously (74). Briefly, reagents (containing 0.0625 μg/μL 3DNA, 0.057 μg/μL IgG, 0.758 μM siRNA) were diluted in PBS and injected intraperitoneally every 3 days for the indicated period of time. For the indicated experiments, AG-120 (Asta Tech, 40817) was suspended in 10% PEG-400, 4% Tween-80, and 86% saline for maximal dissolution of drug. After tumors became palpable (0.6 cm in diameter or 120-150 mm³), the AG-120-containing cocktail was administrated twice daily at 150 mg/kg, with at least 8 hours between doses, unless indicated. For experiments with GSK321 and FSM-3-002, mice were received intraperitoneally at 75 mg/kg, once daily. An equal volume of vehicle was administrated to control mice. NAC water (1.2 g/L) was refreshed twice weekly for the indicated experiments. A high magnesium diet, magnesium sulfate water (MgSO₄, 1.5 g/L) was used for the indicated experiments. For all flank xenograft experiments, tumor volumes were measured twice per week using a caliper (volume=length×width²/2). Upon termination of animal experiments, mice were euthanized using carbon dioxide inhalation followed by cervical dislocation, and tumors were harvested. Body weights were measured once weekly for the indicated time.

¹⁸F-FDG PET/CT Imaging. Mice were fasted five hours prior to ¹⁸F-FDG injection, but had access to water. Subsequently, mice were injected with ¹⁸F-FDG (150-200 uCi/animal). For dynamic scans, mice were scanned for 90 minutes. For static scans, a 30-minute scan was performed 40 minutes after ¹⁸F-FDG injection. Quantitative image analysis of ¹⁸F-FDG uptake in the tumor was performed using Carimas software. The radioactivity data were decay-corrected and normalized to the body weight of the mice, and the amount of ¹⁸F-FDG injected. Radioactivity concentration in the tumor is expressed in terms of standardized uptake value (SUV) as a function of time.

MRI. High resolution T2-weighted MRI images were acquired on a 7T Bruker Biospec MRI scanner. All mice were first anesthetized with isoflurane (2-3% in 100% oxygen gas). The mice were then placed within the MRI scanner and maintained at 35+/−1C and continuously supplied with isoflurane to maintain the respiration rate at 40-60 breaths per minute. Following initial localizer scans, a RARE MRI acquisition was used to acquire high resolution, coronal T2-weighted images (TRITE=4298/35 ms, 37 slices, resolution=500×117×117 microns, matrix=512×256, 2 signal averages). A region-of-interest (ROI) analysis was used to assess tumor volumes in each animal at each timepoint.

Statistical analysis. Data are provided as mean±s.d. from three independent experiments, unless indicated. Median survival was analyzed using the Kaplan-Meier estimate and compared by the log-rank test. P values were calculated using two-tailed, unpaired Student's t-tests using GraphPad Prism 9. *, P<0.05; **, P<0.01; ***, P<0.001.

### Example 3—Results

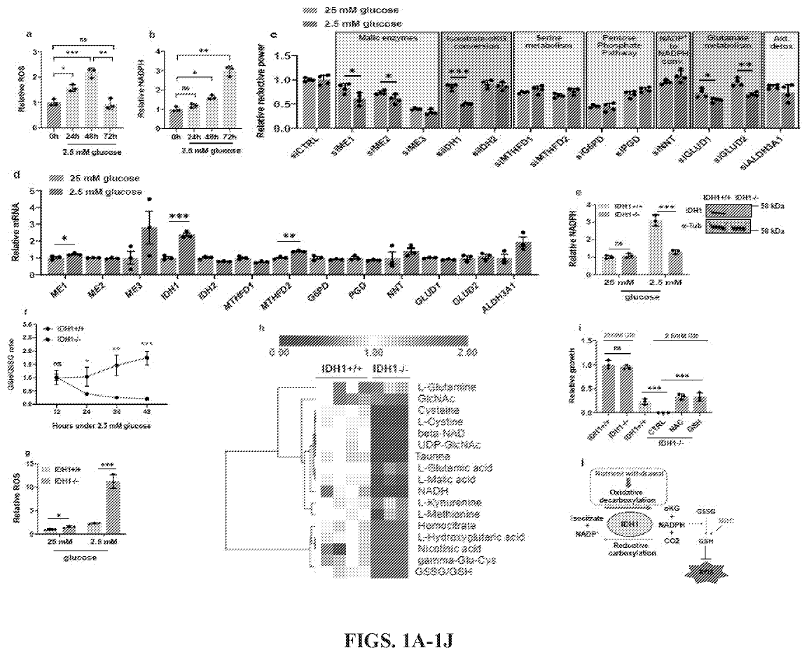

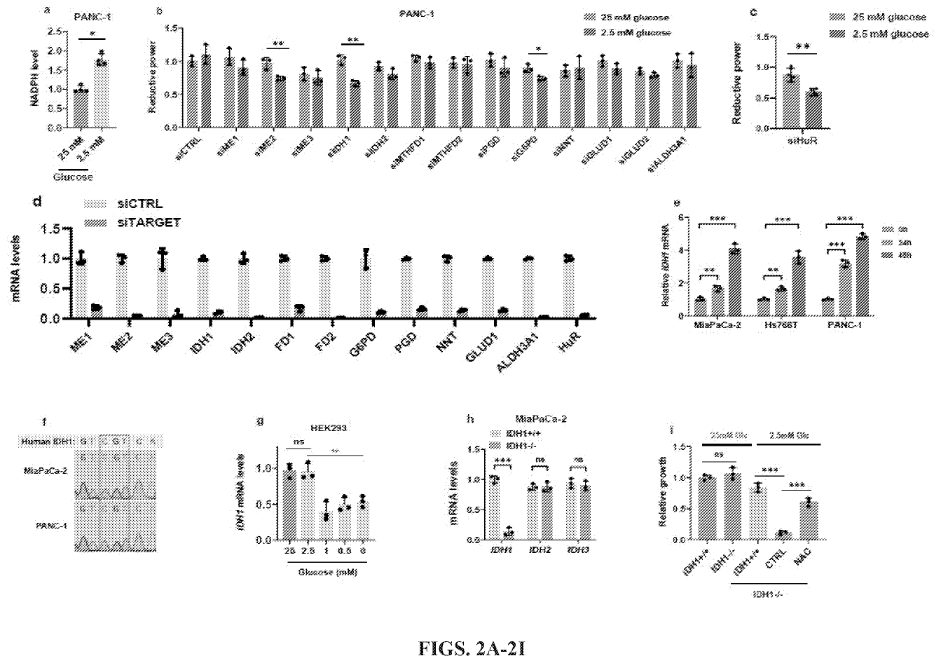

PC cells were cultured under low glucose conditions (2.5 mM) to simulate low levels present in the PC microenvironment (3). A surge in reactive oxygen species (ROS) levels (FIG. 1A) occurred over 48 hours. In multiple cell lines, PC cells quickly compensated with a rise in NADPH (FIG. 1B and FIG. 2A). Consequently, ROS levels returned to baseline the following day (FIG. 1A), revealing maintaining of redox homeostasis under glucose withdrawal. To investigate the role of NADPH-generating enzymes in this adaptive response, an siRNA screen of all 13 NADPH-generating enzymes was performed. Only IDH1 silencing reproducibly lowered reductive power under glucose withdrawal (2.5 mM for 72 hours) in two different PC cell lines (MTT assay normalized to cell number, FIG. 1C, FIG. 2B, and FIG. 2C). Of note, HuR silencing was used as a positive control for ROS induction (6) (FIG. 2D). This unbiased siRNA screen nicely complements the aforementioned studies of HuR's regulation of IDH1 to support antioxidant defense (6, 26). The wild-type sequence was validated in MiaPaCa-2 and PANC-1 human pancreatic cancer cells (FIG. 2F). The role of IDH1 as an acute stress response and survival protein was further demonstrated by the rapid rise in IDH1 mRNA expression upon glucose withdrawal, across multiple PC cell lines (FIG. 2E). These data confirm prior findings by the inventors (6, 28). Moreover, the enzyme proved to be the only NADPH-generating enzyme that responded acutely in this way (FIG. 1D) and highlights why IDH1 is likely to be so crucial under acute metabolic stress. In contrast, IDH1 upregulation did not occur in a non-cancer cell line under glucose withdrawal (FIG. 2G). The observation that cancer cells (but nor non-cancer cells) are especially reliant on IDH1 under nutrient limitation, is an important and recurring theme throughout the present work. Two different IDH1-knockout PC cell lines (IDH1−/−) were generated via CRISPR-Cas9 gene editing. IDH2 and IDH3 mRNA expression were similar between IDH1−/− and isogenic controls (IDH1+/+) (FIG. 2G), revealing a lack of any compensation by other isoforms of the enzyme. Initial studies tested the effects of IDH1 deletion on redox homeostasis. Each set of experiments arrived at IDH1 (FIG. 2F) as an important adaptation under nutrient limitation, through independent approaches. As observed with parental PC cells (FIG. 1A), IDH1+/+ cells adapted well to oxidative stress from glucose withdrawal by upregulation of NADPH and reduced glutathione, however IDH1−/− cells failed to compensate effectively. Impaired redox balance in IDH1−/− cells was corroborated in NADPH (FIG. 1E), GSH/GSSG (FIG. 1F), and ROS levels detected with DCFDA assay (FIG. 1G) assays. The results from an unbiased metabolomics mimicked the findings with isogenic cell lines (FIG. 1H). These experiments revealed redox imbalance in IDH1−/− cells under glucose withdrawal, but not under glucose abundance. Similarly, IDH1-knockout had no effect on cell viability in an in vitro clonogenic assay performed under glucose abundance (e.g., 25 mM). However, IDH1−/− cells derived from two different PC cell lines were crippled under glucose withdrawal. N-acetyl-cysteine (NAC, a glutathione precursor) and reduced glutathione (GSH) rescued IDH1−/− cells under glucose withdrawal in these studies, validating the connection between IDH1 activity and antioxidant defense (FIG. 1I and FIG. 2I). These results collectively indicated that PC cells rely heavily on IDH1-dependent oxidative decarboxylation of IDH1 to maintain redox balance, especially under metabolic stress (FIG. 1J).

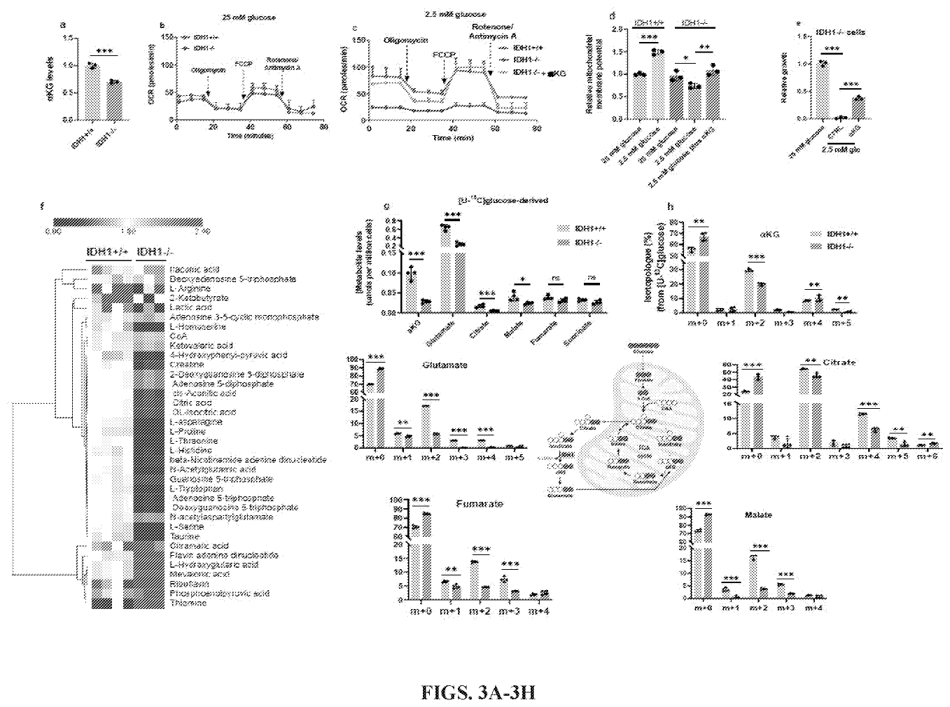

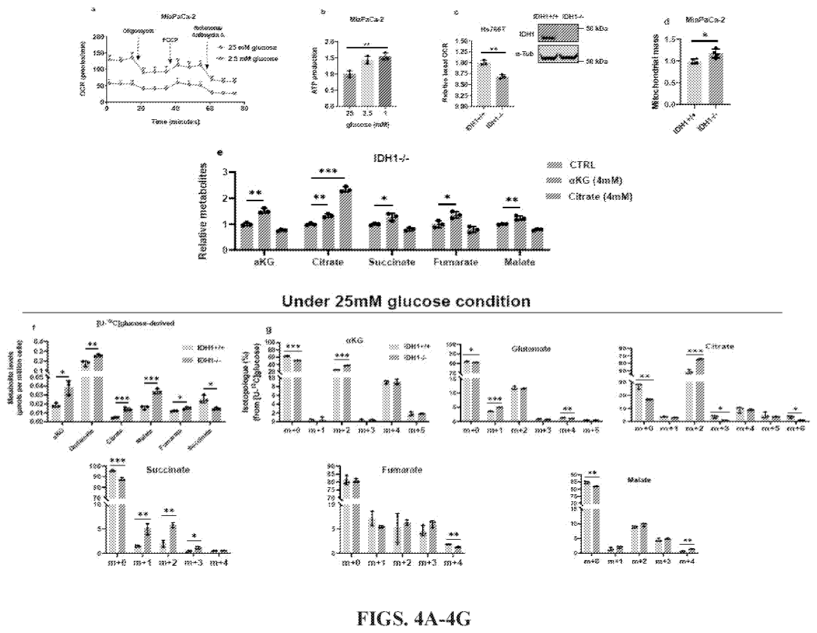

While the impact of IDH1 on redox homeostasis was easy to conceptually connect to changes in NADPH (the established reductive currency in cells), altered alpha-ketoglutarate (αKG) levels (the other product of IDH1-dependent oxidative decarboxylation, FIG. 1J) proved to be equally important, and principally supported mitochondrial metabolism. Metabolic profiling further revealed a reduction in αKG in IDH1−/− cells under glucose withdrawal (FIG. 3A). Numerous prior studies show that cancer cells benefit from increased mitochondrial capacity under nutrient limitation to maximize energy production (15, 16). As expected, parental PC cells (with normal levels of IDH1), adapted to glucose withdrawal by compensatorily increasing oxygen consumption and ATP production (FIGS. 4A & 4B). This finding is consistent with a shift from glycolysis to mitochondrial metabolism under low nutrient conditions to maximize energy production when nutrients are scarce. Similar to previous results from redox assays, there were no differences in oxygen consumption rates (OCR) between IDH1−/− and IDH1+/+PC cells under nutrient replete conditions (FIG. 3B). However, mitochondria in IDH1-knockout cells failed to adapt under low glucose conditions, across diverse PC cell lines (FIGS. 3C, 3D, and 4C). However, these changes were not due to reduction in mitochondrial mass (FIG. 4D). Exogenous αKG nicely rescued mitochondrial oxygen consumption (OCR), mitochondrial membrane potential, and rescue cell growth in these cells (FIGS. 3C-3E). Mitochondrial dysfunction was further validated with metabolic profiling in IDH1−/− cells. A reduction in metabolites related to mitochondrial function was detected in these cells compared to IDH1+/+ cells under nutrient withdrawal (FIG. 3F). In addition, mitochondrial TCA cycle-related metabolites were reduced in IDH1−/− cells under low glucose conditions, as measured through total pool metabolites abundance (all isotopomers) and more detailed ¹³C-enrichment studies, respectively (FIGS. 3G & 3H), indicating carbon flux from [U-¹³C]glucose to mitochondrial TCA metabolites is reduced in IDH1−/− cells, as compared to IDH1+/+ cells. In addition, exogenous αKG rescued metabolite levels of TCA metabolites in IDH1−/− PC cells cultured under low glucose conditions. In contrast, citrate (positioned upstream of the cytosolic wtIDH1 reaction in the cytosol) failed to rescue TCA metabolite levels (FIG. 4E). Consistently, providing IDH1−/− cells with exogenous αKG restored cell viability under the metabolic stress (FIG. 311). These metabolic data spotlight IDH1 as a bona fide metabolic node that heavily influences carbon entry into the TCA cycle by anaplerosis of αKG under low glucose conditions. Through this mechanism, IDH1 supports mitochondrial function to overcome metabolic stress.

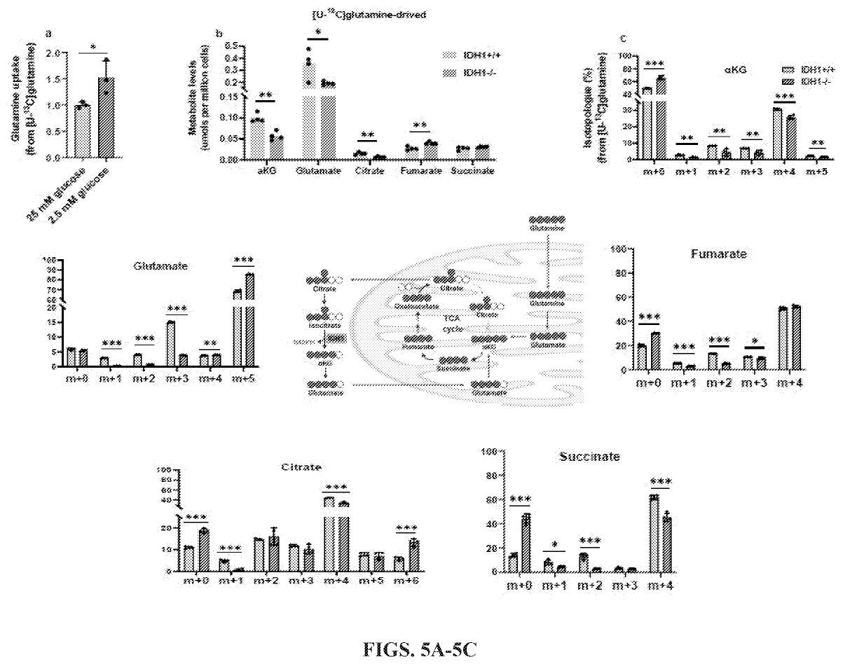

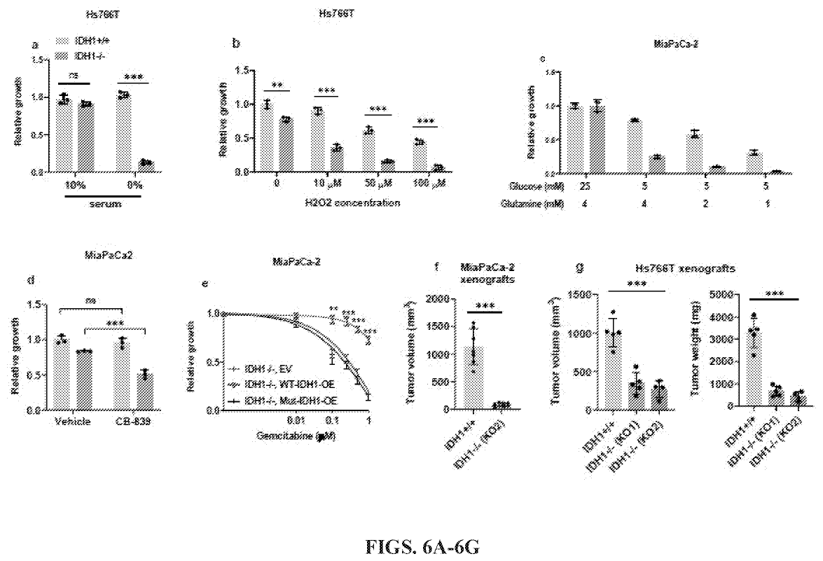

The notion that IDH1 is a key regulator and access point of carbon through central metabolic pathways, and between cellular compartments (mitochondria and cytosol), was further demonstrated through studies of glutamine utilization. Notably, glutamine is a key substrate for αKG through glutaminolysis. This avenue of αKG entry into mitochondrial is likely critical under glucose withdrawal (29-32). Consistent with this concept, the inventors observed increased glutamine uptake in parental PC cells under low glucose conditions (FIG. 5A). Since glutamine is not directly metabolized by IDH1, they did not anticipate a dramatic reduction in ¹³C-flux into the TCA cycle from [U-¹³C]glutamine in IDH1−/− PC cells. However, when IDH1-deficient PC cells were cultured under low glucose conditions, a significant reduction of glutamine-derived carbon was in fact observed in TCA-associated metabolites (FIGS. 5B & 5C). This was most likely attributable to a reduction in TCA cycling, resulting in reduced entry of glutamine-derived carbon to the mitochondria under low glucose conditions. IDH1−/− cells cultured under low glucose conditions were also sensitive to other oxidative insults, such as serum starvation, hydrogen peroxide, glutamine deprivation, a glutaminase inhibitor CB-839, and chemotherapy (FIG. 6A-6E). In the latter experiment, stable re-expression of wtIDH1 (6) rescued IDH1−/− PC cells (i.e., promoted chemotherapy resistance), while the catalytically-altered mtIDH1 did not. These data mirrored a prior published experiment by the inventors where stable reintroduction of the wtIDH1 isoenzyme, after depletion of both IDH1 genes in a heterozygous mtIDH1 tumor, had a far greater impact on tumor proliferation than restored expression of the mutant isoenzyme (26).

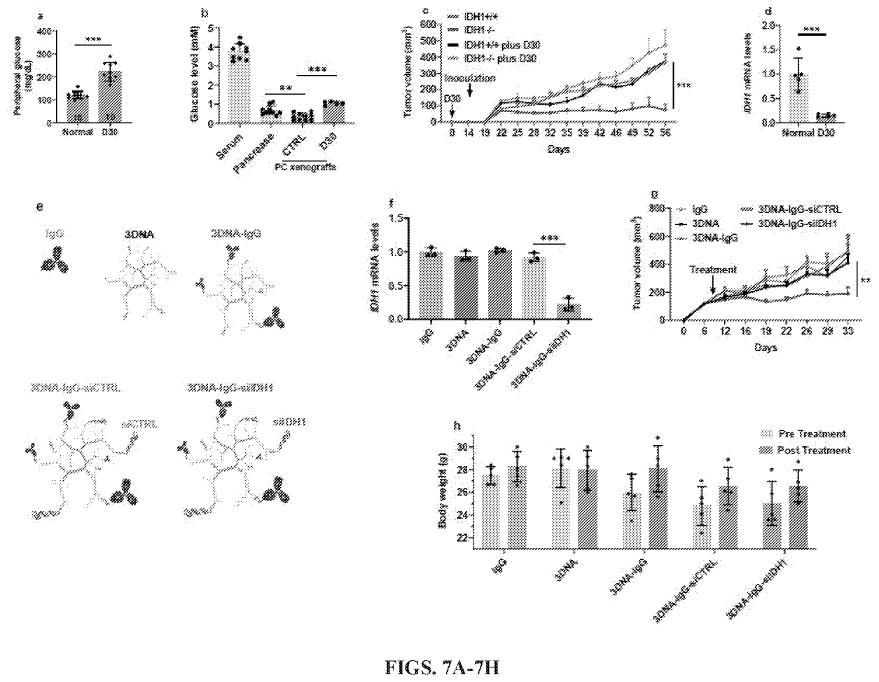

The inventors next speculated that proliferating tumors in vivo would be dependent on wtIDH1, since nutrient gradients developed with greater tumor size (3, 4, 33, 34) Indeed, IDH1−/− xenografts derived from two separate PC cell lines failed to proliferate, as compared to isogenic control xenografts (FIGS. 4F & 4G). In other words, wtIDH1 was required for normal xenograft growth. Next, the inventors tested the hypothesis that the growth of pancreatic tumors is rely on IDH1 under nutrient-derived microenvironment of pancreatic cancer. Forced elevation of peripheral glucose levels by adding glucose to water supply (ad lib consumption of 30% dextrose water; abbreviated as D30 water) resulted in an even greater increase in intra-tumoral glucose levels (FIGS. 7A & 7B). Notably, peripheral glucose levels were only moderately elevated to around 200 mg/dL. Just as with in vitro studies (FIG. 1I), increased intra-tumoral glucose levels rescued IDH1−/− xenograft growth, reinforcing the notion that IDH1 is particularly critical under low glucose conditions (FIG. 7C). Tumors in hyperglycemic mice exhibited a reduction in IDH1 expression (FIG. 7D), presumably related to a diminished dependence on the enzyme under high glucose conditions. In an independent mouse experiment, The inventors also silenced IDH1 using 3DNA nanocarriers. 3DNA is a nanoscale, biodegradable DNA dendrimer with numerous single-stranded oligonucleotide extensions at the periphery of a globular 3DNA structure. These single-stranded oligos are available to hybridize diverse effector molecules through complementary oligonucleotide linker moieties (35). In the present study, 3DNA was derivatized with siRNAs that target the IDH1 transcript (FIG. 7E). Derivatized IgG served as a non-specific ligand molecule that could eventually be replaced with cancer-specific ligands or antibodies for improved cancer cell targeting. When delivered systemically (i.p.), the nanoparticle reduced IDH1 mRNA expression in subcutaneous xenografts, compared to experimental control arms (FIG. 7F). Just as the inventors observed in xenograft studies with IDH1-knockout PC cells, systemic siIDH1 therapy slowed parental PC xenograft growth in mice, as compared to the siRNA control arms (FIG. 7G). Similar to prior studies of a whole-body wtIDH1 knockout mouse (36, 37), the mice showed no adverse physical effects of systemic siIDH1 therapy. Body weights remained stable throughout the experiment (FIG. 7H).

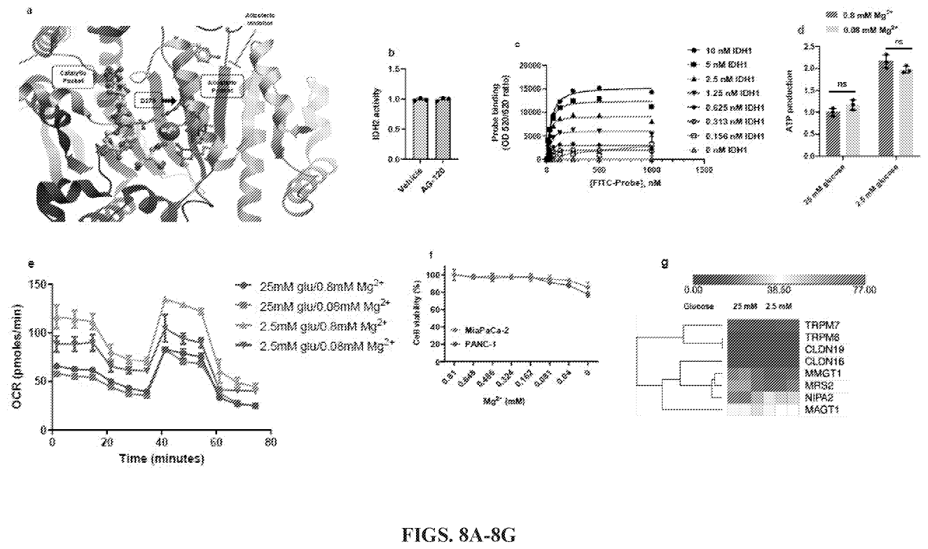

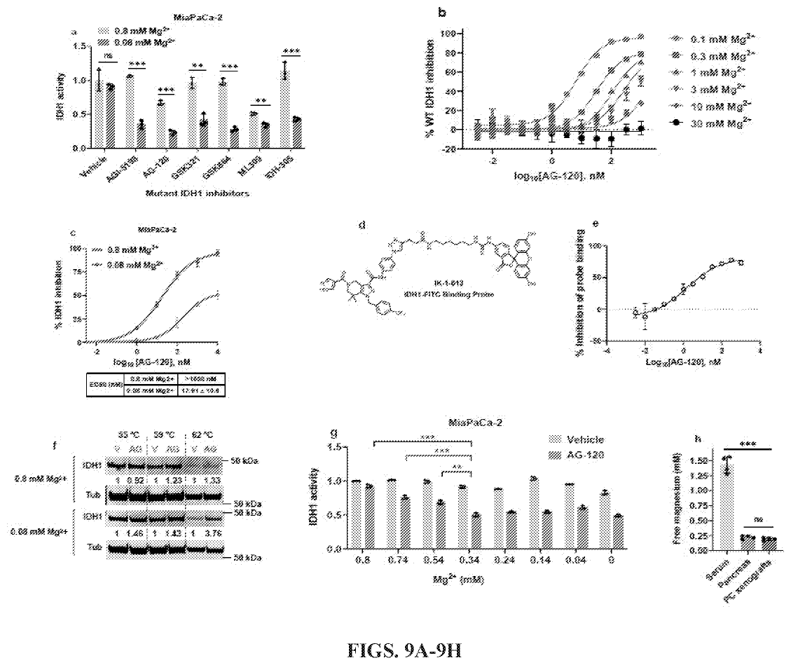

Small molecule inhibitors of mtIDH1 bind the protein at an allosteric site, separate from the catalytic pocket. Under normal conditions, Mg²⁺ interacts with Asp²⁷⁹ in the allosteric pocket to interfere with inhibitor binding at the negatively charged amino acid residue (FIG. 8A) (38). While both isoenzymes (mutant and wild-type) have the same allosteric pocket, there is a widely held belief that Mg²⁺ outcompetes allosteric inhibitors for Asp²⁷⁹ in wtIDH1 due to a lower Km for Mg²⁺ with respect to the wild-type isoenzyme (38, 39). Thus, most allosteric inhibitors of IDH1 are believed to be only weak binders of wtIDH1, but highly selective for the mutant protein (40). However, there has not been a rigorous analysis of the interaction between these compounds in cell-based or in vivo studies. Mg²⁺ levels in tumors are also not well characterized. The possibility remains that these drugs actually target wtIDH1 under physiologic conditions. The magnesium status of tumors is likely much more complex than current considerations suggest (41-47). For instance, studies of Mg²⁺ transporters revealed wide variability in expression and Mg²⁺ transport across different cancers (44). Additionally, much of the intracellular magnesium is protein-bound (48), leaving only a portion available for allosteric modulation of metabolic enzymes (49). In light of this gap in understanding, the inventors examined the efficacy of allosteric IDH1 inhibitors against wtIDH1 in both cell-free and cell-based models, and under varied Mg²⁺ and glucose concentrations. As the literature already suggests (40), conventional allosteric IDH1 inhibitors were generally ineffective against wtIDH1 under standard Mg²⁺ concentrations in cell culture (FIG. 9A). However, every single tested allosteric IDH1 inhibitors turned out to potently inhibit wtIDH1 activity at lower Mg²⁺ concentrations (FIG. 9A). The cell-based assay directly measured NADPH production from isocitrate and NADP+, and therefore provided an assessment of on-target wtIDH1 activity. AG-120 (ivosidenib) was selected for downstream experiments since the drug is very well tolerated in patients and already FDA-approved for medically refractory, IDH1-mutant acute myeloid leukemia (AML) (50). Additionally, AG-120 displayed activity against IDH1-mutant cholangiocarcinoma in a phase III trial (51). The drug was also highly potent in a cell-free wtIDH1 activity assay when Mg²⁺ concentrations were reduced down to 0.1 mM, as compared to higher Mg²⁺ concentrations (FIG. 9B). Importantly, the drug was effective even though Mg²⁺ concentrations used in these experiments were two orders of magnitude above previously reported Km levels of Mg²⁺ for IDH1 (20 μM) (38). WtIDH1 activity was measured in a dose-response experiment of AG-120 in MiaPaCa-2 PC cells cultured in different Mg²⁺ levels. The estimated EC₅₀ after 24 h was >1000 nM and 17.6 nM when cells cultured in 0.8 mM and 0.08 mM, respectively (FIG. 9C). However, AG-120 had no inhibitory activity against the mitochondrial homolog of IDH1, wtIDH2 (FIG. 8B). To further validate whether AG-120 binds wtIDH1, the inventors synthesized the fluorescent probe, IK-1-012, which is structurally similar to GSK321, which has been confirmed to bind in the IDH1 allosteric pocket by X-ray crystallography (52). They determined that IK-1-012 binds to wtIDH1 with a Kd=87 nM and then established an HTRF binding assay to wtIDH1 (FIGS. 9D, 9E, and 8C). Using this assay, they confirmed that AG-120 can competitively displace IK-1-012 and bind to wtIDH1 with an IC₅₀=1.5 nM. Of note, GSK321 binds wtIDH1 with an IC₅₀=1.9 nM. This competitive displacement assay further confirms that AG-120 binds to wtIDH1. A cellular thermal shift assay provided indirect evidence of binding between AG-120 and wtIDH1 (FIG. 9F). At higher temperatures, the compound stabilized the protein under low Mg²⁺ concentrations but had no effect at higher Mg²⁺ levels. In a cell-based wtIDH1 assay with variable Mg²⁺ levels, AG-120 efficacy improved at lower Mg²⁺ concentrations and plateaued at Mg²⁺ levels below 0.4 mM (FIG. 9G). Importantly, these lower concentrations recapitulate conditions in the TME in the in vivo mouse model. To characterize the effects of magnesium levels in cancer cell metabolism, the cells were incubated under various concentration of glucose and magnesium. Reduction in magnesium levels did not cause significant changes in ATP production and OCR (FIG. 8D & 8E), and as baseline control experiment, low Mg²⁺ alone (in the absence of AG-120) had a negligible effect on PC cell growth (FIG. 8F). Notably, pancreatic cancer cells thrive in a microenvironment where both conditions exist. Additionally, incubating cells under low glucose condition did not change the expression of genes involved in magnesium transportation and homeostasis (FIG. 8G). Most important, the concentrations used in the present studies fit nicely within a physiologically relevant Mg²⁺ concentration range observed in tumors, as shown (FIG. 911). Circulating Mg²⁺ levels are roughly 1 mM, and on par with levels in both human sera (53-55) and standard tissue culture media. However, levels in subcutaneous pancreatic tumors were just 20% of these levels (FIG. 911). Of note, Mg²⁺ levels were also reduced in normal tissues in the mouse, which implies that these inhibitors may very well inhibit wtIDH1 throughout the animal, even if the drugs are well-tolerated by the animal. This does not refute clinical experience, which shows a favorable safety profile of these drugs in patients (50). Rather, these data are in lockstep with published reports which reveal that whole-body wtIDH1 knockout mice display a negligible phenotype at baseline (36). Moreover, the observation illustrates a key principle established herein, that wtIDH1 inhibition is highly deleterious to cancer cells which depend on the enzyme for survival under conditions of cancer-associated stress. In contrast, normal cells tolerate wtIDH1 loss or blockade much better, even under low nutrient conditions.

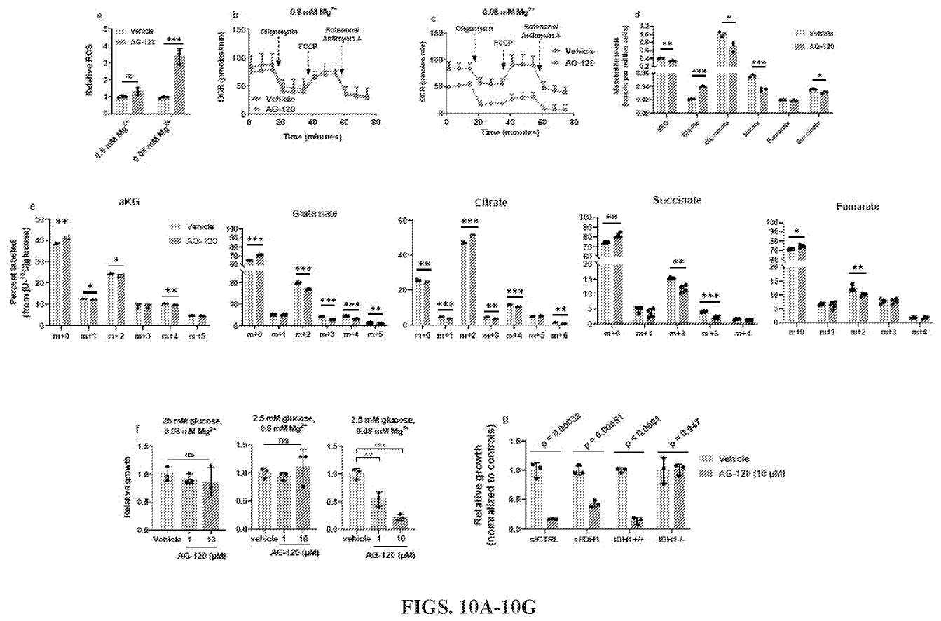

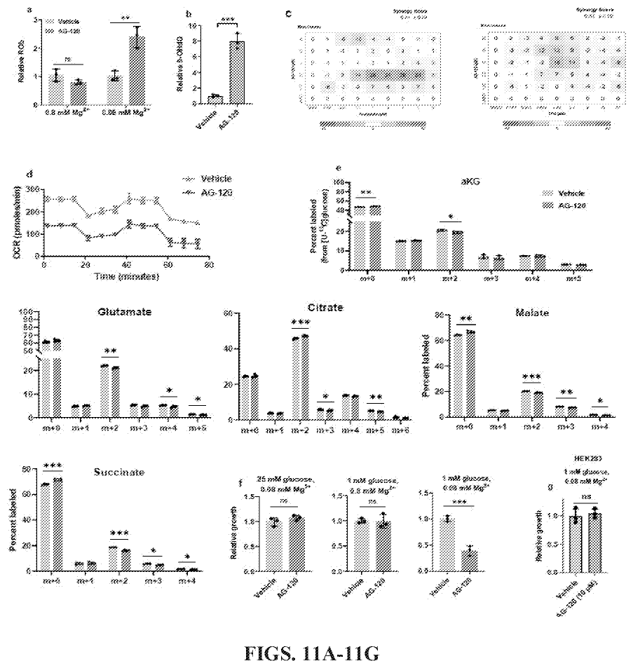

Beyond the inhibitory effects on wtIDH1 activity, allosteric IDH inhibitors exhibit substantial anti-cancer activity under nutrient withdrawal since wtIDH1 is so crucial for survival under these conditions. The inventors first confirmed this through a series of metabolic assays that revealed on-target functional effects of the study drug that mimicked genetic wtIDH1 suppression or ablation. AG-120 treatment under low Mg²⁺, combined with low glucose, resulted in a surge in intracellular ROS. No such effect was observed at higher Mg²⁺ levels (FIG. 10A & FIG. 1A). A similar pattern of oxidative stress under low Mg²⁺ and glucose was even apparent within the nucleus of PC cells, which reflected the broad impact of IDH1 inhibition across cellular compartments (FIG. 11B). Combination index studies revealed that AG-120 enhanced the efficacy of chemotherapeutic agents in PC cells (FIG. 11C).

AG-120 also reduced αKG levels under low glucose conditions (FIG. 10B, similar to IDH1−/− PC cells in FIG. 3B). AG-120 had no effect on oxygen consumption when PC cells were cultured under standard Mg²⁺ concentrations and low glucose (FIG. 10C), but severely impaired oxygen consumption when PC cells were cultured in low Mg²⁺ and glucose (FIGS. 10D & 11D). AG-120 also reduced TCA metabolites levels and enrichment of glucose-derived mitochondrial TCA isotopomers under low glucose and Mg²⁺ concentrations, revealing metabolic impact of pharmacologic IDH1 inhibition previously observed with IDH1−/− PC cells (FIGS. 10D, 10E, & 11E).

The phenotypic and metabolic sequelae of AG-120 treatment translated into a strong therapeutic effect. Human PC cells cultured under low Mg²⁺ and glucose had markedly impaired survival with AG-120 exposure (FIGS. 10F & 11F), but not in non-cancer cells (FIG. 11G). This finding stood in stark contrast to the drug's effect when either Mg²⁺ or glucose levels were maintained at levels typical of standard tissue culture media. Interestingly, cancer cells were resistant to AG-120 when IDH1 expression levels altered. A reduction in AG-120 efficacy was observed when IDH1 expression reduced using siRNA, and the compound was quite ineffective in IDH1−/− cells (FIG. 10G), indicating on-target effects of AG-120 in these cells. A similar result was observed in non-cancer cells, even under low Mg²⁺ and glucose conditions, which highlights the potential therapeutic window for this treatment approach (FIG. 8L).

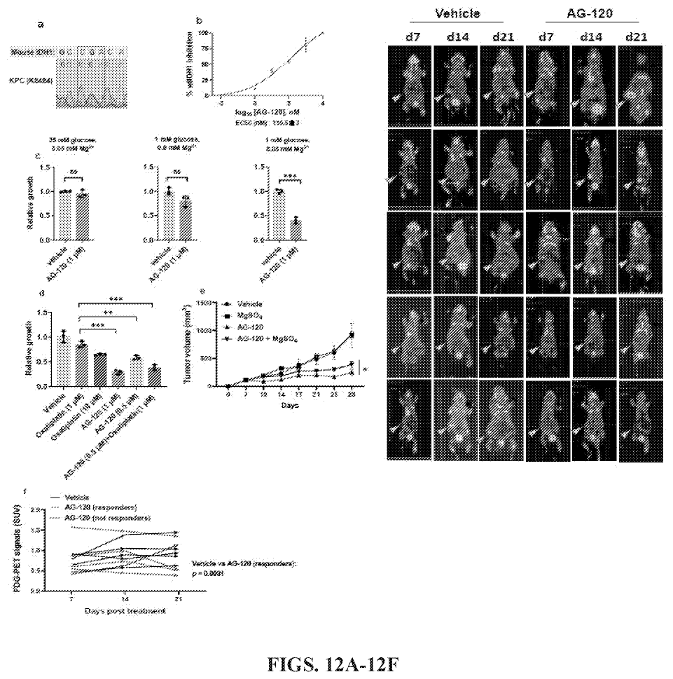

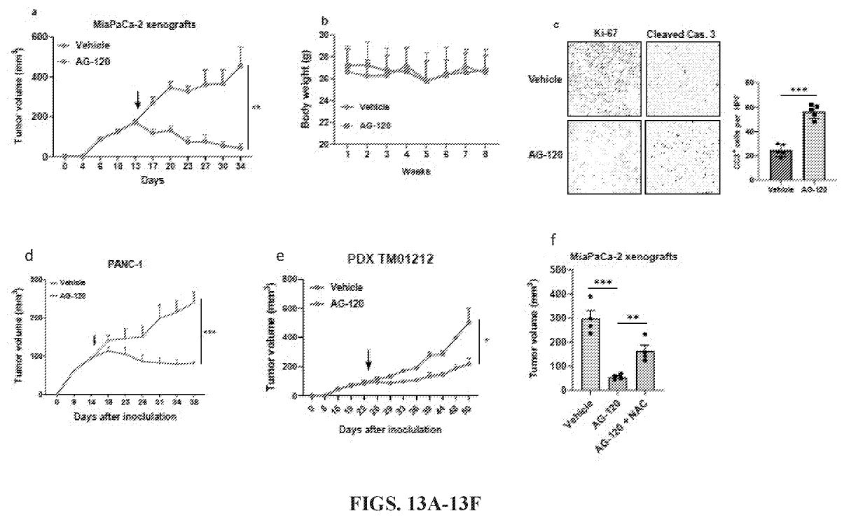

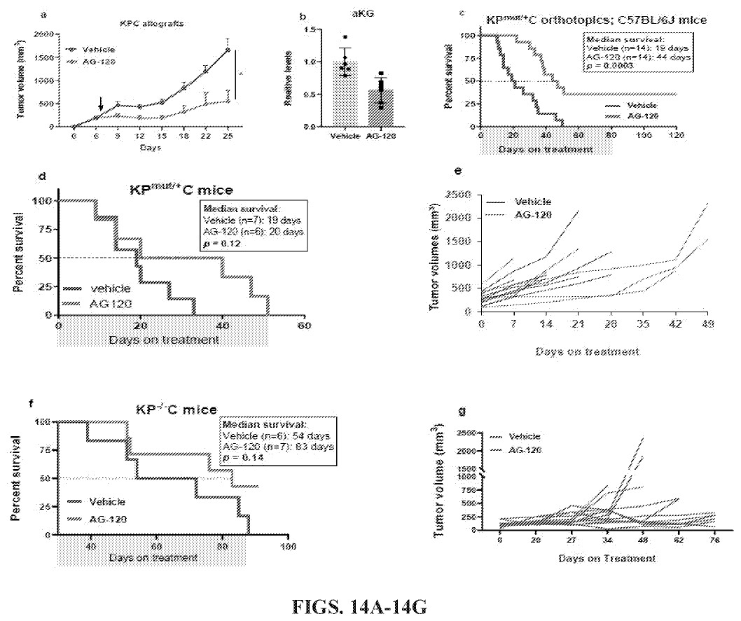

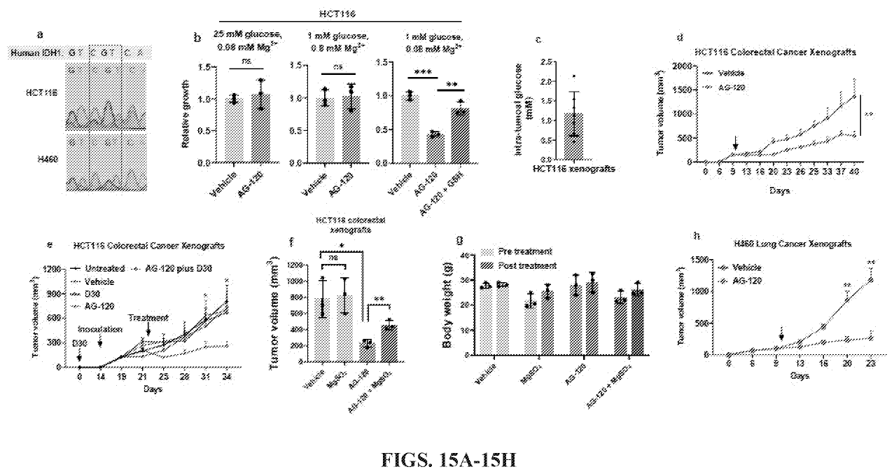

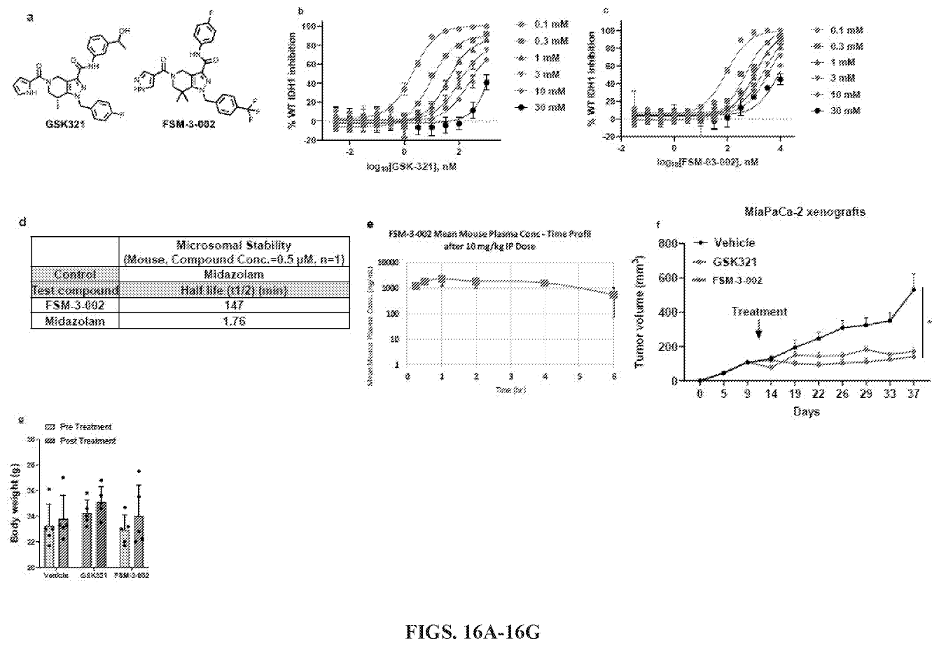

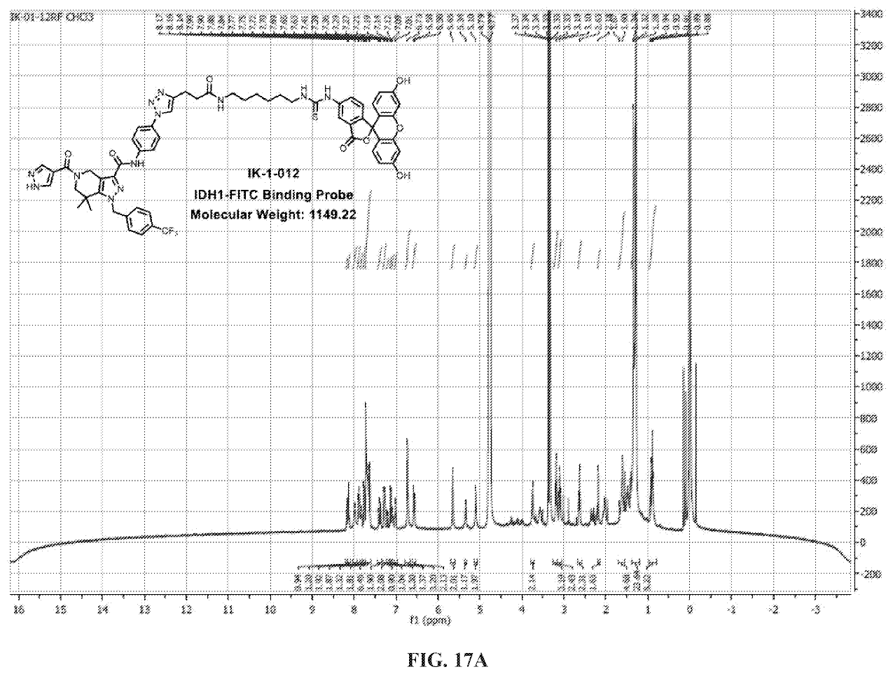

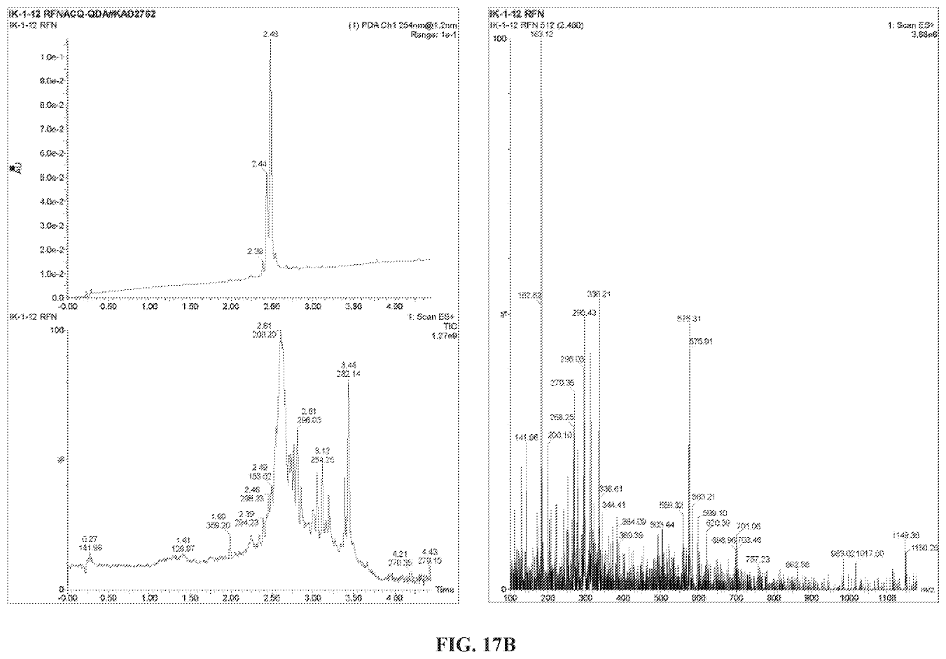

The amino acid sequence is 95.7% conserved across species, accounting for the cross-species activity of the drug. WtIDH1 sequence was confirmed in murine PC cells (KPC K8484) (FIG. 12A). In these cells, AG-120 reduced enzyme activity and cell viability in a similar manner as observed in human PC cells (FIGS. 12B & 12C). These findings distinguish anti-wtIDH1 therapy from conventional chemotherapy in PC models. In the latter scenario, potency unfortunately diminishes with nutrient-deprivation (6). However, AG-120 efficacy increases under these conditions. As an illustration, AG-120 was substantially more potent than the standard-of-care chemotherapeutic (oxaliplatin), when given at the same dose (FIG. 12C). As with the animal experiments earlier testing growth of IDH1−/− cells (FIG. 7A), low intra-tumoral glucose can cause xenograft tumors susceptible to pharmacologic IDH1 inhibition. The dose of AG-120 used for this and subsequent animal experiments was identical to the dose used in prior published mtIDH1 inhibitor studies (150 mg/kg orally twice per day (56, 57)). For the present experiments, human tumors were engrafted into nude mice; murine cancers were generally engrafted into immunocompetent mice (C57BL/6J), unless indicated. AG-120 was highly effective as a single agent against every wtIDH1 pancreatic cancer tested, including multiple human PC cell lines (FIGS. 13A & 13D) and in a human PDX model (FIG. 13E), and animals tolerated the treatment very well (FIG. 13B). Anti-tumor effects of AG-120 were comparable, or even superior (i.e., resulting in tumor shrinkage), to prior reports of the drug against mutant-IDH1 tumors (57). Tumor growth inhibition was confirmed at the cellular level, as shown by a Ki-67 and cleaved caspase-3 immunolabeling assay (FIG. 13C). As with previously described in vitro experiments (FIGS. 1I and 2I), NAC (1.2 g/L water) partially rescued PC growth in AG-120 treated xenografts (FIG. 13F), illustrating the importance of wtIDH1 for antioxidant defense. In addition to human PC models, AG-120 reduced the growth of KPC allografts engrafted subcutaneously into flanks of nude mice (FIG. 14A). Studies to determine AG-120 effectiveness in immunocompetent mice bearing syngeneic orthotopic KPC tumors revealed that the drug was highly effective (FIG. 12E). Moreover, a high magnesium diet (1.5 g/L water) had a partial protective effect (FIG. 12E). This experiment offers direct in vivo evidence that Mg²⁺ blocks AG-120 efficacy in tumors. Metabolic profiling showed that αKG levels are lower in AG-120 treated orthotopic tumors, compared to control animals (FIG. 14B). A survival study in these mice showed that AG-120 improved median survival by over two-fold (19 vs. 44 days) (FIG. 14C). Furthermore, findings were corroborated in a CT/FDG-PET study in mice where treatment reduced intra-tumoral glucose uptake in AG-120 treated mice, compared to control (FIG. 12F), as an independent approach to assess drug efficacy in vivo. Evaluating AG-120 efficacy in two difficult-to-treat murine PC models showed a similar trend, although the outcome did not reach statistical significance (FIGS. 14D-14G). These data raise the possibility that AG-120 may have activity in other types of cancer cells, since most cancers are wild-type for IDH1. As with PC cells, wtIDH1 colon cancer cells (FIG. 15A) responded to the drug under low Mg²⁺ and glucose in culture. In this case, AG-120 treated cancer cells were rescued by a different antioxidant, reduced glutathione (GSH) (FIG. 15B). As with the PC model, subcutaneous HCT116 xenografts also exhibited low intra-tumoral glucose levels (FIG. 15C). Oral AG-120 significantly diminished tumor growth compared to the control arm (FIGS. 15D & 15E). Hyperglycemia induction, by oral D30 water consumption, and providing animals with a high Mg water rescued xenograft growth in AG-120 treated mice, as previously shown for PC IDH1−/− xenografts (FIGS. 7C and 12E). These data reinforced once again that IDH1 represents a metabolic vulnerability under low glucose (i.e., normal intra-tumoral) conditions. All mice tolerated AG-120 treatment extremely well and maintained their weights over time (FIG. 15G).

The inventors also developed and tested a novel wtIDH1 inhibitor by modifying a different mtIDH1 inhibitor, GSK321. GSK321 was reported to have poor bioavailability (52). Therefore, using GSK321 as a lead structure, they initiated a lead optimization study to identify FSM-3-002 (FIG. 16A). As observed with AG-120, the compounds are potent wtIDH1 inhibitors under low Mg levels (FIGS. 16B & 16C). The inventors evaluated FSM-3-002 in mouse liver microsomes as a measure of metabolic stability which is predictive of in vivo bioavailability. A similar analog to GSK321 containing the metabolically labile pyrrole ring showed poor metabolic stability in the mouse liver microsome assay with a short half-life (t_{1/2}˜4 min). FSM-3-002 is an analog which was identified using a bio-isosteric approach to replace the pyrrole with a more stable motif and was found to have superior metabolic stability, compared to pyrrole containing GSK321 analogs, in the mouse liver microsome assay with a t_{1/2}=147 min (FIG. 16D). The inventors then evaluated FSM-3-002 in a mouse pharmacokinetics study in CD-1 mice by intraperitoneal (IP) administration at a 10 mg/kg dose at 6 time points (0.25, 0.5, 1, 2, 4, and 6 hours). FSM-3-002 was shown to have good overall plasma exposure with a Cmax=2325 ng/mL at 1 hour (FIG. 16E). The compound was well-tolerated in mice and caused a marked reduction in MiaPaCa2 xenograft growth (FIGS. 16F & 16G).

### Example 4—Discussion

Wild-type IDH1 was introduced as a potential therapeutic target in cancer during the last decade. Metallo et al., demonstrated that reductive carboxylation of glutamine-derived αKG by wtIDH1 supported lipogenesis in melanoma cells cultured under hypoxia (23). The authors showed that suppression of the enzyme reduced cell growth. Similar biochemistry was observed under normoxia in cancer types with deficient mitochondria, including an osteosarcoma and a renal cell carcinoma cell line (22, 58). The same group also showed the importance of reductive carboxylation by wtIDH1 for spheroid growth in an in vitro lung cancer model (21). More recently, Calvert et al. determined that the reverse wtIDH1 reaction, oxidative decarboxylation, supported glioma growth through the production of NADPH, and that this biology served to control ROS. Some of these studies employed mtIDH1 inhibitors (GSK321 and the related GSK864 which have mild wtIDH1 activity) as pharmacologic ‘tools’ to test the impact of wtIDH1 inhibition in vitro and other pre-clinical models (21, 24, 52). Studies of whole-body wtIDH1 knockout revealed that wtIDH1 deletion did not affect mouse wellness at baseline, but mice were vulnerable to oxidative liver injury with sub-lethal doses of LPS (36, 37). This very small, but illuminating group of studies, establish several important principles related to wtIDH1: 1) the enzyme is a promising therapeutic target worthy of further study, and 2) inhibition of the wtIDH1 target should have a reasonable safety profile against non-cancer tissues.

The present study builds on these prior works by offering several novel insights. First, the inventors show that the cancer microenvironment raises the importance of wtIDH1 for cancer cell survival, particularly in comparison to the enzyme's role in normal cells. In numerous cell culture and animal experiments, wtIDH1 proved expendable to cancer cells under nutrient-replete conditions. Neither genetic deletion, nor pharmacologic inhibition affected PC viability or metabolism when ambient glucose concentrations were high. This finding raises the probability that glycemic status predicts response to anti-wtIDH1 therapy. This in turn would provide a rationale for tight glucose control in diabetic patients receiving anti-wtIDH1 therapy. Furthermore, the enzyme was expendable to non-cancer cells, even under austere conditions. This was not only true in cell culture experiments, but also in mouse tissues where the inventors unexpectedly observed both low glucose and Mg²⁺ levels compared to serum. Yet, no drug toxicity was observed, either in these experiments or prior reports (50, 57, 59). Perhaps increased wtIDH1 dependence in cancer cells (vs. normal tissues) is attributable to the incessant exposure of cancer cells to numerous oxidative threats beyond low glucose, including low levels of other nutrients (60-62), hypoxia (21, 23, 25, 63, 64), and cytotoxic immune cells (20, 65-67). Even further, cancer cells are likely more fragile and susceptible to oxidative stress than normal cells due to their aneuploid state (68, 69). Regardless, wtIDH1 is overexpressed in cancer tissues (6, 24) and the enzyme appears to represent a bona fide metabolic vulnerability to cancer cells that exist in low nutrient conditions, with a promising therapeutic window.

Second, the inventors elucidate mechanistically that IDH1 supports PC cell fitness under nutrient limitation by enhancing antioxidant defense and mitochondrial function. These adaptive survival strategies are mediated by the two products of wtIDH1 oxidative decarboxylation: NADPH and αKG. While prior studies demonstrate that wtIDH1 is protective against ROS (6, 21, 24), the present work also introduces the notion that the cytosolic enzyme regulates TCA cycle activity through αKG-mediated anaplerosis, and in so doing, wtIDH1 inhibition adversely impacts mitochondrial function. These mechanistic insights lay the foundation for rational combination therapies that cooperate to impose lethal metabolic stress on cancer cells in the context of nutrient limitation. Combined wtIDH1 and glutaminase inhibition in this study offers just one proof-of-concept example.

Third and most importantly, the inventors discovered that allosteric IDH1 inhibitors, which were designed specifically to target mtIDH1, hold promise as therapeutics against the 99% of cancers that lack the intended therapeutic target. They demonstrated that under low ambient Mg²⁺ levels, wtIDH1 is highly susceptible to inhibitory effects of these drugs. Indeed, off-the-shelf allosteric inhibitors exhibited tremendous potency at nanomolar concentrations under low Mg²⁺ conditions. In the context of low nutrient levels present in the TME, these drugs therefore exhibit strong anti-cancer activity by blocking wtIDH1 activity required for cancer cell survival. Oral administration of the FDA-approved mtIDH1 inhibitor, AG-120, effectively treated numerous and diverse wtIDH1 cancers in mice. The drug even increased survival in an orthotopic PC model by an enormous amount compared to historical experiments evaluating other promising compounds (4, 70, 71). Importantly, drug dosing used here were the same as previous studies of mtIDH1 tumors, yet the outcomes in the present study were more favorable (50, 51, 56, 57).

Taken together, these data reveal that two principal conditions are required for AG-120 efficacy against wtIDH1 cancer. First, reduced Mg²⁺ ensures drug activity against wtIDH1. Second, nutrient limitation (ubiquitous in tumors like PC) elevates cancer cell reliance on wtIDH1, and renders tumors vulnerable to the drug. If either condition is absent, the drug is not effective. However, when both conditions are present, cancer cells are highly susceptible to IDH inhibitors. In contrast, wtIDH1 appears to be expendable in normal tissues, where the drug is ineffective, even under harsh metabolic conditions (summarized in FIG. 14I). This work provides a foundation for the immediate implementation of a clinical trial testing AG-120 as a monotherapy or in combination with chemotherapy against wtIDH1 cancers. Additionally, optimization of anti-wtIDH1 therapy is now possible based on mechanistic insights gained from this work.

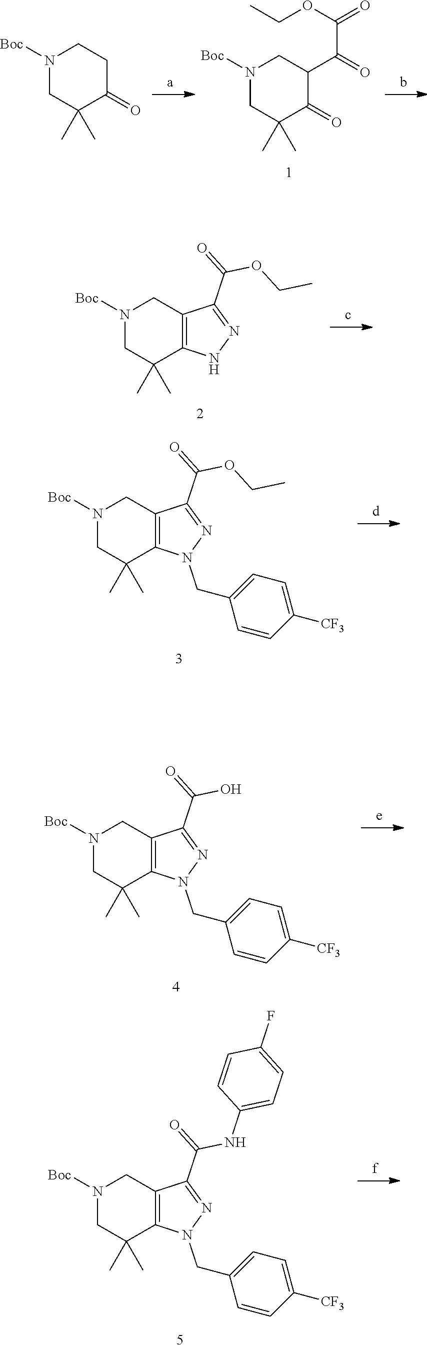 [SMILES: CC(C)(C)OC(=O)N1CCC(=O)C(C)(C)C1 ; CCOC(=O)C(=O)C1CN(C(=O)OC(C)(C)C)CC(C)(C)C1=O ; CCOC(=O)c1n[nH]c2c1CN(C(=O)OC(C)(C)C)CC2(C)C ; CCOC(=O)c1nn(Cc2ccc(C(F)(F)F)cc2)c2c1CN(C(=O)OC(C)(C)C)CC2(C)C ; CC(C)(C)OC(=O)N1Cc2c(C(=O)O)nn(Cc3ccc(C(F)(F)F)cc3)c2C(C)(C)C1 ; CC(C)(C)OC(=O)N1Cc2c(C(=O)Nc3ccc(F)cc3)nn(Cc3ccc(C(F)(F)F)cc3)c2C(C)(C)C1]

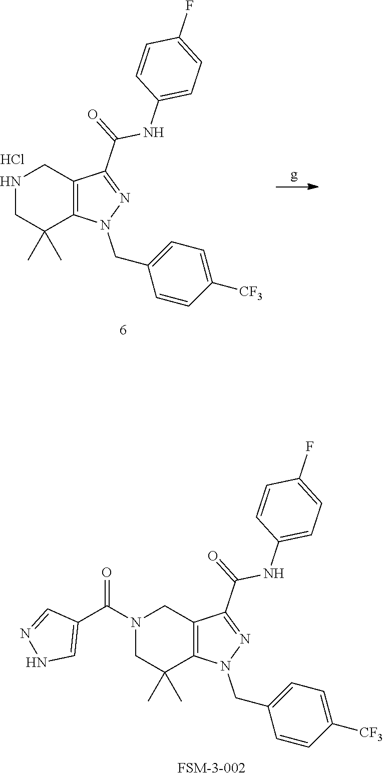 [SMILES: Cl ; CC1(C)CNCc2c(C(=O)Nc3ccc(F)cc3)nn(Cc3ccc(C(F)(F)F)cc3)c21 ; CC1(C)CN(C(=O)c2cn[nH]c2)Cc2c(C(=O)Nc3ccc(F)cc3)nn(Cc3ccc(C(F)(F)F)cc3)c21]

Reagents and conditions: (a) Diethyl oxalate, LDA, anhydrous THF, −78° C. -r.t; 8 h (b) hydrazine hydrate, Acetic acid, rt, 8 h; (c) 1-(Bromomethyl)-4-(trifluoromethyl)benzene, NaH, anhydrous THF, 0° C. -rt, 8 h; (d) NaOH, Ethanol:H₂O (3:1), rt; 4 h; (e) 4-Fluoroaniline, HATU, DIPEA, DMF, rt, 1 h; (f) 4N HCl in dioxane, dcm, 0° C.-rt, 2 h; (g) 1H-Pyrazole-4-carboxylic acid, EDCI, HOBt, DIPEA, THF, rt, 6 h.

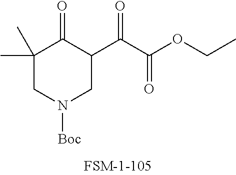 [SMILES: CCOC(=O)C(=O)C1CN(C(=O)OC(C)(C)C)CC(C)(C)C1=O]

tert-Butyl 5-(2-ethoxy-2-oxoacetyl)-3,3-dimethyl-4-oxopiperidine-1-carboxylate: To a stirred solution of tert-butyl 3,3-dimethyl-4-oxopiperidine-1-carboxylate (20 g, 88 mmol) in anhydrous THE (250 mL) was added LDA (2.0 M THE 50.6 mL, 101.2 mmol) at −78° C. dropwise. The reaction mixture was stirred at −78° C. for one hour and a solution of diethyl oxalate (15.57 g, 106.48 mmol) in anhydrous THE (50 mL) was added. The resulting reaction mixture was warmed to room temperature and stirred overnight. After completion of the reaction as indicated by TLC, water (500 mL) was added. The aqueous phase was neutralized with 1 N HCl and extracted with ethyl acetate (4×150 mL). The organic layer was washed with brine (500 mL), dried over Na₂SO₄, concentrated under reduced pressure and the crude residue was purified by flash chromatography using (0-20% ethyl acetate in hexanes). Roto evaporation yielded the pure compound 1 (16.97 g, 51.9 mmol, 51.8% yield) as a yellow oil. ¹H NMR (400 MHz, CDCl₃) δ 4.46 (s, 2H), 4.36 (d, J 6.9 Hz, 2H), 3.40 (s, 1H), 1.59 (s, 2H), 1.49 (d, J=9.9 Hz, 9H), 1.39 (t, J=7.0 Hz, 3H), 1.21 (s, 6H). MS (ESI): m/z 350.32 [M+23]⁺.

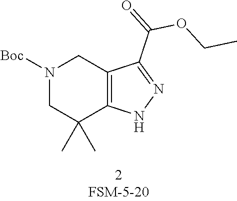 [SMILES: CCOC(=O)c1n[nH]c2c1CN(C(=O)OC(C)(C)C)CC2(C)C]

5-tert-Butyl 3-ethyl 7,7-dimethyl-6,7-dihydro-1H-pyrazolo[4,3-c]pyridine-3,5(4H)-dicarboxylate: To a solution of tert-butyl 5-(2-ethoxy-2-oxoacetyl)-3,3-dimethyl-4-oxopiperidine-1-carboxylate (16.97 g, 51.9 mmol) in acetic acid (40 mL) was added hydrazine hydrate (4 mL, 124 mmol) portion wise at room temperature. The mixture was stirred at room temperature overnight then poured into ice cold saturated aqueous sodium bicarbonate and extracted with ethyl acetate (3×150 mL). The combined organic layer was washed with brine (300 mL) and dried over Na₂SO₄ and evaporated under vacuum. The crude residue was purified by flash chromatography using (0-25% ethyl acetate in hexanes) to provide the desired compound 2 (14.6 g, 46.4 mmol, 89.3% yield) as a white solid. ¹H NMR (400 MHz, CDCl₃) δ 4.66 (d, J=17.8 Hz, 2H), 4.37 (d, J=6.3 Hz, 2H), 3.42 (s, 2H), 1.50 (s, 9H), 1.38 (s, 3H), 1.29 (d, J=10.2 Hz, 6H). MS (ES): m/z 324.22 [M+1]⁺.

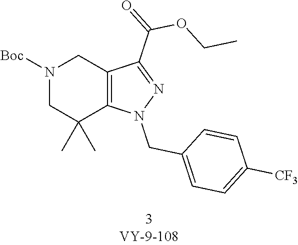 [SMILES: CCOC(=O)c1nn(Cc2ccc(C(F)(F)F)cc2)c2c1CN(C(=O)OC(C)(C)C)CC2(C)C]

5-tert-Butyl 3-ethyl 7,7-dimethyl-1-(4-(trifluoromethyl)benzyl)-6,7-dihydro-1H-pyrazolo[4,3-c]pyridine-3,5(4H)-dicarboxylate: To a solution of 5-tert-butyl 3-ethyl 7,7-dimethyl-6,7-dihydro-1H-pyrazolo[4,3-c]pyridine-3,5(4H)-dicarboxylate (10.94 g, 33.9 mmol) in anhydrous THF (150 mL) was slowly added 60% sodium hydride (1.63 g, 40.6 mmol) at room temperature. After 1 hour, a solution of 1-(bromomethyl)-4-trifluoromethylbenzene (6.72 g, 35.6 mmol) in anhydrous THF (5 mL) was added and the mixture was stirred at room temperature overnight. The reaction mixture was quenched with water (100 mL) and the aqueous layer was extracted with ethyl acetate (3×100 mL). The combined organic layer was washed with brine (200 mL), dried over sodium sulfate, filtered, and concentrated under reduced pressure. The crude residue was purified by flash chromatography using (0-25% ethyl acetate in hexanes) to provide the desired compound 3 (7.2 g, 16.8 mmol, 50.0% yield) as a yellow oil. ¹H NMR (400 MHz, CDCl₃) δ 7.58 (d, J=8.2 Hz, 22H), 7.16 (d, J=3.4 Hz, 2H), 5.55 (s, 2H), 4.67 (d, J=21.3 Hz, 2H), 4.12 (q, J=7.1 Hz, 2H), 3.39 (s, 2H), 1.48 (s, 9H), 1.17 (s, 6H). MS (ES): m/z 482.25 [M+1]⁺.

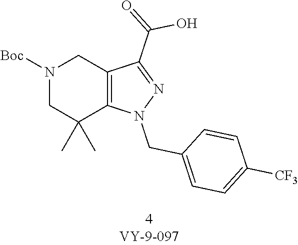 [SMILES: CC(C)(C)OC(=O)N1Cc2c(C(=O)O)nn(Cc3ccc(C(F)(F)F)cc3)c2C(C)(C)C1]

5-(tert-butoxycarbonyl)-7,7-dimethyl-1-(4-(trifluoromethyl)benzyl)-4,5,6,7-tetrahydro-1H-pyrazolo[4,3-c]pyridine-3-carboxylic acid: To a solution of 5-tert-butyl 3-ethyl 7,7-dimethyl-1-(4-(trifluoromethyl)benzyl)-6,7-dihydro-1H-pyrazolo[4,3-c]pyridine-3,5(4H)-dicarboxylate (4.3 g, 10 mmol) in EtOH (40 mL) was added sodium hydroxide (0.8 g, 20 mmol) in water (20 mL). The resulting mixture was stirred at room temperature for 4 hours, concentrated under reduced pressure, diluted with water (40 mL) and washed with ethyl acetate (100 mL). The pH of the aqueous layer was adjusted to 6 with 1 N HCl and the resulting precipitate was collected by filtration and dried to give desired compound 4 (3.03 g, 7.52 mmol, 75% yield) as a white solid. VY-10-097: ¹H NMR (400 MHz, DMSO) δ 7.70 (d, J=8.2 Hz, 2H), 7.25 (d, J=7.8 Hz, 2H), 5.58 (s, 2H), 4.51 (s, 2H), 3.30 (s, 2H), 1.41 (s, 9H), 1.11 (s, 6H MS (ESI): m/z 454.25 [M+1]⁺.

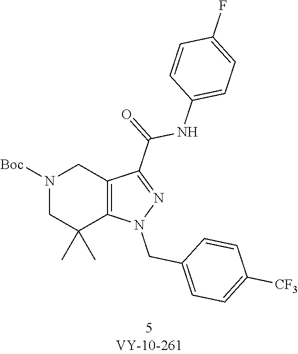 [SMILES: CC(C)(C)OC(=O)N1Cc2c(C(=O)Nc3ccc(F)cc3)nn(Cc3ccc(C(F)(F)F)cc3)c2C(C)(C)C1]

tert-butyl 3-(4-fluorophenylcarbamoyl)-7,7-dimethyl-1-(4-(trifluoromethyl)benzyl)-6,7-dihydro-1H-pyrazolo[4,3-c]pyridine-5(4H)-carboxylate: To a stirred solution of 5-(tert-butoxycarbonyl)-7,7-dimethyl-1-(4-(trifluoromethyl)benzyl)-4,5,6,7-tetrahydro-1H-pyrazolo[4,3-c]pyridine-3-carboxylic acid (0.95 g, 2.11 mmol) in anhydrous DMF (10 mL) was added HATU (1.60 g, 4.22 mmol) and after stirring the mixture for 5 min DIPEA (0.98 g, 7.59 mmol) was added. The reaction mixture was then stirred for an additional 10-15 min. Then 4-fluoroaniline (0.26 g, 2.32 mmol) was added. The reaction mixture was stirred at room temperature for an additional 2 hours. After completion of the reaction, as monitored by tlc, water was added to the reaction mixture, and the aqueous layer was extracted with ethyl acetate (3×50 mL). The combined organic layer was washed with brine, dried over sodium sulfate, and evaporated under vacuum. The crude residue was purified by flash chromatography using (0-50% EA in hexanes) to obtain the pure compound (1.01 g, 88.01% yield) as a white solid. ¹H NMR (400 MHz, CDCl₃) δ 8.56 (s, 1H), 7.65-7.57 (m, 4H), 7.14 (d, J=7.3 Hz, 2H), 7.03 (t, J=8.6 Hz, 2H), 5.51 (s, 2H), 4.75 (s, 2H), 3.41 (s, 2H), 1.48 (s, 9H), 1.20 (s, 6H).MS (ESI): m/z 569.28 [M+23]⁺.

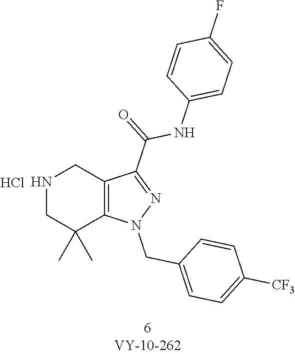 [SMILES: Cl ; CC1(C)CNCc2c(C(=O)Nc3ccc(F)cc3)nn(Cc3ccc(C(F)(F)F)cc3)c21]

N-(4-fluorophenyl)-7,7-dimethyl-1-(4-(trifluoromethyl) benzyl)-4,5,6,7-tetrahydro-1H-pyrazolo[4,3-c]pyridine-3-carboxamide hydrochloride(6): To a solution of compound 4, tert-butyl 3-(4-fluorophenylcarbamoyl)-7,7-dimethyl-1-(4-(trifluoromethyl)benzyl)-6, 7-dihydro-1H-pyrazolo[4,3-c]pyridine-5 (4H)-carboxylate (1.01 g, 1.856 mmol) in 10 ml dichloromethane, was added 10 ml of 4N HCl in dioxane slowly at 0° C. The mixture was stirred at room temperature for 2-3 h. after starting material disappeared reaction mixture concentrated under vacuum, toluene was added to precipitated white solid several times and evaporated under vacuum to remove traces of dioxane, and finally formed solid was filtered, washed with 10% ethyl acetate/hexane, and dried over vacuum to obtain desired compound 6 (890 mg, 99.5%) as HCl salt. ¹H NMR (400 MHz, DMSO) δ 10.32 (s, 1H), 9.66 (s, 2H), 7.85-7.78 (m, 2H), 7.75 (d, J=8.2 Hz, 2H), 7.33 (d, J=8.1 Hz, 2H), 7.25-7.05 (m, 2H), 5.73 (s, 2H), 4.31 (s, 2H), 3.21 (s, 2H), 1.35 (s, 6H). MS (ESI): m/z 447.34 [M+1]⁺.

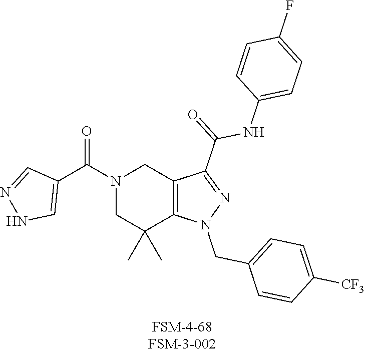 [SMILES: CC1(C)CN(C(=O)c2cn[nH]c2)Cc2c(C(=O)Nc3ccc(F)cc3)nn(Cc3ccc(C(F)(F)F)cc3)c21]

N-(4-Fluorophenyl)-7,7-dimethyl-5-(1H-pyrazole-4-carbonyl)-1-(4-(trifluoromethyl)benzyl)-4,5,6,7-tetrahydro-1H-pyrazolo[4,3-c]pyridine-3-carboxamide: To a stirred solution of 1H-pyrazole-4-carboxylic acid (0.212 g, 1.90 mmol) in anhydrous DMF (10 mL) was added EDCI (0.327 g, 2.85 mmol) and after stirring the mixture for 5 min, HOBt (384 mg, 2.85 mmol), DIPEA (0.575 g, 5.7 mmol) was added. The reaction mixture was then stirred for an additional 10-15 min. Then N-(4-fluorophenyl)-7,7-dimethyl-1-(4-(trifluoromethyl)benzyl)-4, 5,6,7-tetrahydro-1H-pyrazolo[4,3-c]pyridine-3-carboxamide hydrochloride (0.890 mg, 1.84 mmol) was added. The reaction mixture was stirred at room temperature for an additional 8 hours. After completion of the reaction, as monitored by tlc, water was added to the reaction mixture, and the aqueous layer was extracted with ethyl acetate (3×50 mL). The combined organic layer was washed with brine, dried over sodium sulfate, and evaporated under vacuum. The crude residue was purified by flash chromatography using (0-90% EA in hexanes) to obtain the pure compound (780 mg, 78.2% yield) as white solid. ¹H NMR (400 MHz, CDCl₃) δ 8.53 (s, 1H), 7.97 (s, 2H), 7.62 (d, J=8.2 Hz, 2H), 7.57 (dd, J=8.8, 4.7 Hz, 2H), 7.16 (d, J=8.2 Hz, 2H), 7.04 (t, J=8.7 Hz, 2H), 5.52 (s, 2H), 5.09 (s, 2H), 3.75 (s, 2H), 1.28 (s, 6H). ¹³C NMR (101 MHz, DMSO) δ 164.22, 160.85, 159.90, 157.52, 147.04, 142.85, 141.09, 139.66, 135.37, 135.34, 130.60, 128.98, 128.67, 128.35, 128.04, 127.58, 126.01, 125.98, 125.94, 123.30, 122.79, 122.71, 120.60, 116.40, 115.69, 115.61, 115.39, 54.13, 34.08, 25.73, 25.55, 25.13. (¹⁹F splitting present). MS (ES): m/z 541.27 [M+1]⁺.

Similarly, a fluorophore conjugated compound was also prepared using a azide substituted phenyl ring to link to fluorophore as shown in the Scheme below. These compounds were characterized in FIGS. 9A & 9B.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Anderson, Practical Process Research & Development—A Guide for Organic Chemists, 2^{nd} ed., Academic Press, New York, 2012.
Handbook of Pharmaceutical Salts: Properties, and Use, Stahl and Wermuth Eds., Verlag Helvetica Chimica Acta, 2002.
Reagan-Shaw et al., FASEB J., 22(3):659-661, 2008.
Smith, March's Advanced Organic Chemistry: Reactions, Mechanisms, and Structure, 7^{th} Ed., Wiley, 2013.
1. A. Vaziri-Gohar, M. Zarei, J. R. Brody, J. M. Winter, Metabolic Dependencies in Pancreatic Cancer. Frontiers in oncology 8, 617 (2018).
2. C. J. Whatcott et al., Desmoplasia in Primary Tumors and Metastatic Lesions of Pancreatic Cancer. Clinical cancer research: an official journal of the American Association for Cancer Research 21, 3561-3568 (2015).
3. J. J. Kamphorst et al., Human pancreatic cancer tumors are nutrient poor and tumor cells actively scavenge extracellular protein. Cancer research 75, 544-553 (2015).
4. K. P. Olive et al., Inhibition of Hedgehog signaling enhances delivery of chemotherapy in a mouse model of pancreatic cancer. Science 324, 1457-1461 (2009).
5. M. A. Badgley et al., Cysteine depletion induces pancreatic tumor ferroptosis in mice. Science 368, 85-89 (2020).
6. M. Zarei et al., Posttranscriptional Upregulation of IDH1 by HuR Establishes a Powerful Survival Phenotype in Pancreatic Cancer Cells. Cancer research 77, 4460-4471 (2017).
7. D. E. Biancur, A. C. Kimmelman, The plasticity of pancreatic cancer metabolism in tumor progression and therapeutic resistance. Biochim Biophys Acta Rev Cancer (2018).
8. C. A. Lyssiotis, L. C. Cantley, Targeting metabolic scavenging in pancreatic cancer. Clin Cancer Res 20, 6-8 (2014).
9. C. M. Sousa, A. C. Kimmelman, The complex landscape of pancreatic cancer metabolism. Carcinogenesis 35, 1441-1450 (2014).
10. A. C. Kimmelman, Metabolic Dependencies in RAS-Driven Cancers. Clin Cancer Res 21, 1828-1834 (2015).
11. D. E. Biancur et al., Functional Genomics Identifies Metabolic Vulnerabilities in Pancreatic Cancer. Cell Metab 33, 199-210.e198 (2021).
12. S. Yang et al., Pancreatic cancers require autophagy for tumor growth. Genes Dev 25, 717-729 (2011).
13. C. Commisso et al., Macropinocytosis of protein is an amino acid supply route in Ras-transformed cells. Nature 497, 633-637 (2013).
14. C. M. Sousa et al., Pancreatic stellate cells support tumour metabolism through autophagic alanine secretion. Nature 536, 479-483 (2016).
15. R. Rossignol et al., Energy substrate modulates mitochondrial structure and oxidative capacity in cancer cells. Cancer research 64, 985-993 (2004).
16. K. Birsoy et al., Metabolic determinants of cancer cell sensitivity to glucose limitation and biguanides. Nature 508, 108-112 (2014).
17. I. M. Ahmad et al., Mitochondrial 02*- and H₂O₂ mediate glucose deprivation-induced stress in human cancer cells. J Biol Chem 280, 4254-4263 (2005).
18. G. M. DeNicola et al., Oncogene-induced Nrf2 transcription promotes ROS detoxification and tumorigenesis. Nature 475, 106-109 (2011).
19. M. G. Vander Heiden, L. C. Cantley, C. B. Thompson, Understanding the Warburg effect: the metabolic requirements of cell proliferation. Science 324, 1029-1033 (2009).
20. C. Feig et al., The pancreas cancer microenvironment. Clin Cancer Res 18, 4266-4276 (2012).
21. L. Jiang et al., Reductive carboxylation supports redox homeostasis during anchorage-independent growth. Nature 532, 255-258 (2016).
22. A. R. Mullen et al., Reductive carboxylation supports growth in tumour cells with defective mitochondria. Nature 481, 385-388 (2011).
23. C. M. Metallo et al., Reductive glutamine metabolism by IDH1 mediates lipogenesis under hypoxia. Nature 481, 380-384 (2011).
24. A. E. Calvert et al., Cancer-Associated IDH1 Promotes Growth and Resistance to Targeted Therapies in the Absence of Mutation. Cell Rep 19, 1858-1873 (2017).
25. D. R. Wise et al., Hypoxia promotes isocitrate dehydrogenase-dependent carboxylation of α-ketoglutarate to citrate to support cell growth and viability. Proc Natl Acad Sci USA 108, 19611-19616 (2011).
26. M. Zarei et al., RNA-Binding Protein HuR Regulates Both Mutant and Wild-Type IDH1 in IDH1-Mutated Cancer. Mol Cancer Res 17, 508-520 (2019).
27. S. E. Weinberg, N. S. Chandel, Targeting mitochondria metabolism for cancer therapy. Nature chemical biology 11, 9-15 (2015).
28. R. A. Burkhart et al., HuR is a post-transcriptional regulator of core metabolic enzymes in pancreatic cancer. RNA biology 10, 1312-1323 (2013).
29. D. R. Wise, C. B. Thompson, Glutamine addiction: a new therapeutic target in cancer. Trends Biochem Sci 35, 427-433 (2010).
30. A. J. Bott et al., Glutamine Anabolism Plays a Critical Role in Pancreatic Cancer by Coupling Carbon and Nitrogen Metabolism. Cell Rep 29, 1287-1298.e1286 (2019).
31. R. J. DeBerardinis, T. Cheng, Q's next: the diverse functions of glutamine in metabolism, cell biology and cancer. Oncogene 29, 313-324 (2010).
32. C. T. Hensley, A. T. Wasti, R. J. DeBerardinis, Glutamine and cancer: cell biology, physiology, and clinical opportunities. J Clin Invest 123, 3678-3684 (2013).
33. R. M. Sutherland, Cell and environment interactions in tumor microregions: the multicell spheroid model. Science 240, 177-184 (1988).
34. P. P. Provenzano et al., Enzymatic targeting of the stroma ablates physical barriers to treatment of pancreatic ductal adenocarcinoma. Cancer cell 21, 418-429 (2012).
35. Y. H. Huang et al., Delivery of Therapeutics Targeting the mRNA-Binding Protein HuR Using 3DNA Nanocarriers Suppresses Ovarian Tumor Growth. Cancer research 76, 1549-1559 (2016).
36. M. Itsumi et al., Idh1 protects murine hepatocytes from endotoxin-induced oxidative stress by regulating the intracellular NADP(+)/NADPH ratio. Cell Death Differ 22, 1837-1845 (2015).
37. J. Ye et al., IDH1 deficiency attenuates gluconeogenesis in mouse liver by impairing amino acid utilization. Proceedings of the National Academy of Sciences of the United States of America 114, 292-297 (2017).
38. G. Deng et al., Selective inhibition of mutant isocitrate dehydrogenase 1 (IDH1) via disruption of a metal binding network by an allosteric small molecule. J Biol Chem 290, 762-774 (2015).
39. X. Xie et al., Allosteric Mutant IDH1 Inhibitors Reveal Mechanisms for IDH1 Mutant and Isoform Selectivity. Structure 25, 506-513 (2017).
40. D. J. Urban et al., Assessing inhibitors of mutant isocitrate dehydrogenase using a suite of pre-clinical discovery assays. Scientific reports 7, 12758 (2017).
41. A. Fuhrmann, A. Banisadr, P. Beri, T. D. Tlsty, A. J. Engler, Metastatic State of Cancer Cells May Be Indicated by Adhesion Strength. Biophys J 112, 736-745 (2017).
42. M. H. Seltzer, F. E. Rosato, M. J. Fletcher, Serum and tissue magnesium levels in human breast carcinoma. J Surg Res 10, 159-162 (1970).
43. V. Digiesi, R. Bandinelli, P. Bisceglie, E. Santoro, Magnesium in tumoral tissues, in the muscle and serum of subjects suffering from neoplasia. Biochem Med 29, 360-363 (1983).
44. F. I. Wolf, V. Trapani, Magnesium and its transporters in cancer: a novel paradigm in tumour development. Clin Sci (Loud) 123, 417-427 (2012).
45. F. I. Wolf, A. R. Cittadini, J. A. Maier, Magnesium and tumors: ally or foe? Cancer Treat Rev 35, 378-382 (2009).
46. A. Nasulewicz et al., Magnesium deficiency inhibits primary tumor growth but favors metastasis in mice. Biochim Biophys Acta 1739, 26-32 (2004).
47. W. Q. Huang et al., Direct and indirect associations between dietary magnesium intake and breast cancer risk. Sci Rep 9, 5764 (2019).
48. J. Vormann, Magnesium: Nutrition and Homoeostasis. AIMS Public Health 3, 329-340 (2016).
49. F. I. Wolf, TRPM7: channeling the future of cellular magnesium homeostasis? Sci STKE 2004, pe23 (2004).
50. C. D. DiNardo et al., Durable Remissions with Ivosidenib in IDH1-Mutated Relapsed or Refractory AML. N Engl J Med 378, 2386-2398 (2018).
51. G. K. Abou-Alfa et al., Ivosidenib in IDH1-mutant, chemotherapy-refractory cholangiocarcinoma (ClarIDHy): a multicentre, randomised, double-blind, placebo-controlled, phase 3 study. Lancet Oncol 21, 796-807 (2020).
52. U. C. Okoye-Okafor et al., New IDH1 mutant inhibitors for treatment of acute myeloid leukemia. Nature chemical biology 11, 878-886 (2015).
53. F. W. Lowenstein, M. F. Stanton, Serum magnesium levels in the United States,
1971-1974. J Am Coll Nutr 5, 399-414 (1986).
54. R. B. Costello et al., Perspective: The Case for an Evidence-Based Reference Interval for Serum Magnesium: The Time Has Come. Adv Nutr 7, 977-993 (2016).
55. J. R. Weisinger, E. Bellorin-Font, Magnesium and phosphorus. Lancet 352, 391-396 (1998).
56. J. Popovici-Muller et al., Discovery of AG-120 (Ivosidenib): A First-in-Class Mutant IDH1 Inhibitor for the Treatment of IDH1 Mutant Cancers. ACS Med Chem Lett 9, 300-305 (2018).
57. B. Nicolay, R. Narayanaswamy, E. Aguado. (Presented at the 22nd Annual Scientific Meeting and Education Day of the Society for Neuro-oncology, 2017).
58. A. R. Mullen et al., Oxidation of alpha-ketoglutarate is required for reductive carboxylation in cancer cells with mitochondrial defects. Cell Rep 7, 1679-1690 (2014).
59. S. Dhillon, Ivosidenib: First Global Approval. Drugs 78, 1509-1516 (2018).
60. J. Garcia-Bermudez, R. T. Williams, R. Guarecuco, K. Birsoy, Targeting extracellular nutrient dependencies of cancer cells. Mol Metab 33, 67-82 (2020).
61. Z. T. Schug et al., Acetyl-CoA synthetase 2 promotes acetate utilization and maintains cancer cell growth under metabolic stress. Cancer Cell 27, 57-71 (2015).
62. K. Izuishi, K. Kato, T. Ogura, T. Kinoshita, H. Esumi, Remarkable tolerance of tumor cells to nutrient deprivation: possible new biochemical target for cancer therapy. Cancer Res 60, 6201-6207 (2000).
63. A. R. Anderson, A. M. Weaver, P. T. Cummings, V. Quaranta, Tumor morphology and phenotypic evolution driven by selective pressure from the microenvironment. Cell 127, 905-915 (2006).
64. A. C. Koong et al., Pancreatic tumors show high levels of hypoxia. Int J Radiat Oncol Biol Phys 48, 919-922 (2000).
65. A. Ene-Obong et al., Activated pancreatic stellate cells sequester CD8+ T cells to reduce their infiltration of the juxtatumoral compartment of pancreatic ductal adenocarcinoma. Gastroenterology 145, 1121-1132 (2013).
66. J. L. Carstens et al., Spatial computation of intratumoral T cells correlates with survival of patients with pancreatic cancer. Nat Commun 8, 15095 (2017).
67. E. Karamitopoulou, Tumour microenvironment of pancreatic cancer: immune landscape is dictated by molecular and histopathological features. Br J Cancer 121, 5-14 (2019).
68. L. Zhou, L. J. Jilderda, F. Foijer, Exploiting aneuploidy-imposed stresses and coping mechanisms to battle cancer. Open Biol 10, 200148 (2020).
69. D. L. Newman, S. L. Gregory, Co-Operation between Aneuploidy and Metabolic Changes in Driving Tumorigenesis. Int J Mol Sci 20, (2019).
70. T. M. Nywening et al., Targeting both tumour-associated CXCR2. Gut 67, 1112-1123 (2018).
71. Z. D'Costa et al., Gemcitabine-Induced TIMIP1 Attenuates Therapy Response and Promotes Tumor Growth and Liver Metastasis in Pancreatic Cancer. Cancer Res 77, 5952-5962 (2017).
72. D. Martinez Molina et al., Monitoring drug target engagement in cells and tissues using the cellular thermal shift assay. Science 341, 84-87 (2013).
73. S. R. Hingorani et al., Trp53R172H and KrasG12D cooperate to promote chromosomal instability and widely metastatic pancreatic ductal adenocarcinoma in mice. Cancer Cell 7, 469-483 (2005).
74. L. M. Merlo, J. Bowers, T. Stefanoni, R. Getts, L. Mandik-Nayak, B-Cell-Targeted 3DNA Nanotherapy Against Indoleamine 2,3-Dioxygenase 2 (IDO2) Ameliorates Autoimmune Arthritis in a Preclinical Model. Clin Pathol 13, 2632010X20951812 (2020).

What is claimed is:

1. A compound of the formula: 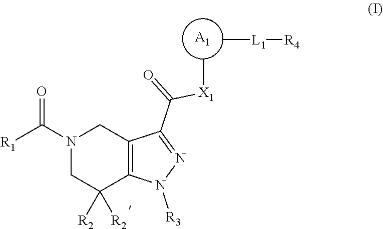
wherein:
A₁ is arenediyl_{(C≤12)}, substituted arenediyl_{(C≤12)}, heteroarenediyl_{(C≤12)}, or substituted heteroarenediyl_{(C≤12)};
L₁ is a covalent bond, —O—, —S—, —NR_{b}—, —C(O)—, —OC(O)—, —C(O)O—, —C(O)NR_{b}—, —NR_{b}C(O)—, —OC(O)NR_{b}—, —NR_{b}C(O)O—, —OC(O)O—, —NR_{b}C(O)NR_{b}—, -heteroarenediyl_{(C≤12)}-alkanediyl_{(C≤12)}-, or substituted -heteroarenediyl_{(C≤12)}-alkanediyl_{(C≤12)}-; wherein R_{b} is hydrogen, alkyl_{(C≤12)}, or substituted alkyl_{(C≤12)};
R₁ is aryl_{(C≤12)}, substituted aryl_{(C≤12)}, heteroaryl_{(C≤12)}, or substituted heteroaryl_{(C≤12)};
R₂ and R₂′ are each independently alkyl_{(C≤12)} or substituted alkyl_{(C≤12)};
R₃ is aralkyl_{(C≤12)}, substituted aralkyl_{(C≤12)}, heteroaralkyl_{(C≤12)}, or substituted heteroaralkyl_{(C≤12)};
R₄ is hydrogen, amino, cyano, halo, hydroxy, or nitro; or
alkyl_{(C≤12)}, cycloalkyl_{(C≤12)}, alkenyl_{(C≤12)}, alkynyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino_{(C≤12)}, amido_{(C≤12)}, or a substituted version of any of these groups; or
-L₂-alkanediyl_{(C≤12)}-L₃-R₅, wherein:
L₂ and L₃ is —C(O)—, —OC(O)—, —C(O)O—, —NRₐC(O)—, —NRₐC(O)—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein:
Rₐ and Rₐ′ are each independently hydrogen, alkyl_{(C≤12)}, or substituted alkyl_{(C≤12)};
R₅ is a ubiquitin ligase ligand or a fluorophore; and
X₁ is —O— or —NR_{b}—, wherein:
R_{b} is hydrogen, alkyl_{(C≤12)}, or substituted alkyl_{(C≤12)};
or a pharmaceutically acceptable salt thereof.

2. The compound of claim 1, wherein the compound is further defined as: 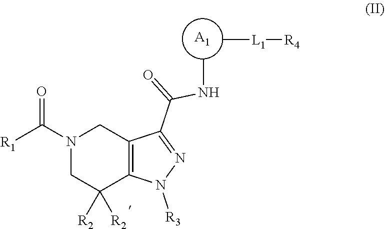
wherein:
A₁ is arenediyl_{(C≤12)}, substituted arenediyl_{(C≤12)}, heteroarenediyl_{(C≤12)}, or substituted heteroarenediyl_{(C≤12)};
L₁ is a covalent bond, —O—, —S—, —NR_{b}—, —C(O)—, —OC(O)—, —C(O)O—, —C(O)NR_{b}—, —NR_{b}C(O)—, —OC(O)NR_{b}—, —NR_{b}C(O)O—, —OC(O)O—, —NR_{b}C(O)NR_{b}—, -heteroarenediyl_{(C≤12)}-alkanediyl_{(C≤12)}-, or substituted -heteroarenediyl_{(C≤12)}-alkanediyl_{(C≤12)}-; wherein R_{b} is hydrogen, alkyl_{(C≤12)}, or substituted alkyl_{(C≤12)};
R₁ is aryl_{(C≤12)}, substituted aryl_{(C≤12)}, heteroaryl_{(C≤12)}, or substituted heteroaryl_{(C≤12)};
R₂ and R₂′ are each independently alkyl_{(C≤12)} or substituted alkyl_{(C≤12)};
R₃ is aralkyl_{(C≤12)}, substituted aralkyl_{(C≤12)}, heteroaralkyl_{(C≤12)}, or substituted heteroaralkyl_{(C≤12)}; and
R₄ is hydrogen, amino, cyano, halo, hydroxy, or nitro; or
alkyl_{(C≤12)}, cycloalkyl_{(C≤12)}, alkenyl_{(C≤12)}, alkynyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino _{(C≤12)}, amido_{(C≤12)}, or a substituted version of any of these groups; or
-L₂-alkanediyl_{(C≤12)}-L₃-R₅, wherein:
L₂ and L₃ is —C(O)—, —OC(O)—, —C(O)O—, —NRₐC(O)—, —NRₐC(O)—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein:
Rₐ and R_{a′} are each independently hydrogen, alkyl_{(C≤12)}, or substituted alkyl_{(C≤12)};
R₅ is a ubiquitin ligase ligand or a fluorophore; and
or a pharmaceutically acceptable salt thereof.

3. The compound of claim 1, wherein the compound is further defined as: 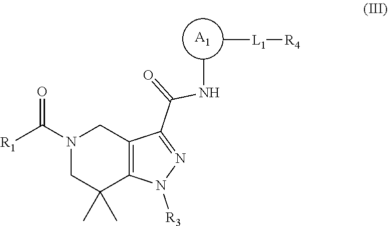
wherein:
A₁ is arenediyl_{(C≤12)}, substituted arenediyl_{(C≤12)}, heteroarenediyl_{(C≤12)}, or substituted heteroarenediyl_{(C≤12)};
L₁ is a covalent bond, —O—, —S—, —NR_{b}—, —C(O)—, —OC(O)—, —C(O)O—, —C(O)NR_{b}—, —NR_{b}C(O)—, —OC(O)NR_{b}—, —NR_{b}C(O)O—, —OC(O)O—, —NR_{b}C(O)NR_{b}—, -heteroarenediyl_{(C≤12)}-alkanediyl_{(C≤12)}-, or substituted -heteroarenediyl_{(C≤12)}-alkanediyl_{(C≤12)}-; wherein R_{b} is hydrogen, alkyl_{(C≤12)}, or substituted alkyl_{(C≤12)};
R₁ is aryl_{(C≤12)}, substituted aryl_{(C≤12)}, heteroaryl_{(C≤12)}, or substituted heteroaryl_{(C≤12)};
R₃ is aralkyl_{(C≤12)}, substituted aralkyl_{(C≤12)}, heteroaralkyl_{(C≤12)}, or substituted heteroaralkyl_{(C≤12)}; and
R₄ is hydrogen, amino, cyano, halo, hydroxy, or nitro; or
alkyl_{(C≤12)}, cycloalkyl_{(C≤12)}, alkenyl_{(C≤12)}, alkynyl_{(C≤12)}, heterocycloalkyl_{(C≤12)}, alkoxy_{(C≤12)}, cycloalkoxy_{(C≤12)}, alkylamino_{(C≤12)}, dialkylamino_{(C≤12)}, cycloalkylamino_{(C≤12)}, dicycloalkylamino _{(C≤12)}, amido_{(C≤12)}, or a substituted version of any of these groups; or
-L₂-alkanediyl_{(C≤12)}-L₃-R₅, wherein:
L₂ and L₃ is —C(O)—, —OC(O)—, —C(O)O—, —NRₐC(O)—, —C(O)NRₐ—, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein:
Rₐ and Rₐ′ are each independently hydrogen, alkyl_{(C≤12)}, or substituted alkyl_{(C≤12)};
R₅ is a ubiquitin ligase ligand or a fluorophore; and
or a pharmaceutically acceptable salt thereof.

4. The compound according to any one of claims 1-3, wherein R₃ is aralkyl_{(C≤12)} or substituted aralkyl_{(C≤12)}.

5. The compound according to any one of claims 1-4, wherein R₃ is substituted aralkyl_{(C≤12)}.

6. The compound according to any one of claims 1-5, wherein R₃ is 4-fluorobenzyl or 4-trifluoromethylbenzyl.

7. The compound according to any one of claims 1-6, wherein R₁ is heteroaryl_{(C≤12)} or substituted heteroaryl_{(C≤12)}.

8. The compound according to any one of claims 1-7, wherein R₁ is heteroaryl_{(C≤12)}.

9. The compound according to any one of claims 1-8, wherein R₁ is 2-pyrrolyl, 4-pyrazolyl, 5-pyrazolyl, 2-thiophenyl, N-methylpyrrol-2-yl, or 4-methylpyrrol-2-yl.

10. The compound according to any one of claims 1-9, wherein A₁ is arenediyl_{(C≤12)} or substituted arenediyl_{(C≤12)}.

11. The compound according to any one of claims 1-10, wherein A₁ is arenediyl_{(C≤12)}.

12. The compound according to any one of claims 1-10, wherein A₁ is 1,3-benzenediyl or 1,4-benzenediyl.

13. The compound according to any one of claims 1-9, wherein A₁ is heteroarenediyl_{(C≤12)} or substituted heteroarenediyl_{(C≤12)}.

14. The compound according to any one of claims 1-9 and 13, wherein A₁ is heteroarenediyl_{(C≤12)}.

15. The compound according to any one of claims 1-9, 13, and 14, wherein A₁ is oxazol-2,4-diyl, oxazol-2,5-diyl, thiazol-2,4-diyl, or thiazol-2,5-diyl.

16. The compound according to any one of claims 1-15, wherein L₁ is a covalent bond.

17. The compound according to any one of claims 1-15, wherein L₁ is —O—.

18. The compound according to any one of claims 1-15, wherein L₁ is —C(O)—.

19. The compound according to any one of claims 1-15, wherein L₁ is —NR_{b}C(O)— or —C(O)NR_{b}—.

20. The compound of claim 19, wherein L₁ is —NR_{b}C(O)—.

21. The compound according to any one of claims 1-15, 19, and 20, wherein R_{b} is hydrogen.

22. The compound according to any one of claims 1-15, wherein L₁ is -heteroarenediyl_{(C≤12)}-alkanediyl_{(C≤12)}- or substituted -heteroarenediyl_{(C≤12)}-alkanediyl_{(C≤12)}-.

23. The compound of claim 22, wherein L₁ is -heteroarenediyl_{(C≤12)}-alkanediyl_{(C≤12)}-.

24. The compound of either claim 22 or claim 23, wherein heteroarenediyl_{(C≤12)} of L₁ is 1,4-triazoldiyl.

25. The compound according to any one of claims 22-24, wherein the alkanediyl_{(C≤12)} of L₁ is ethylene.

26. The compound according to any one of claims 1-18, wherein R₄ is hydrogen.

27. The compound according to any one of claims 1-18, wherein R₄ is hydroxy.

28. The compound according to any one of claims 1-18, wherein R₄ is amino.

29. The compound according to any one of claims 1-18, wherein R₄ is halo.

30. The compound according to any one of claims 1-18 and 27, wherein R₄ is fluoro.

31. The compound according to any one of claims 1-18, wherein R₄ is alkoxy_{(C≤12)} or substituted alkoxy_{(C≤12)}.

32. The compound according to any one of claims 1-18 and 31, wherein R₄ is alkoxy_{(C≤12)}.

33. The compound according to any one of claims 1-18, 31, and 32, wherein R₄ is methoxy.

34. The compound according to any one of claims 1-18, wherein R₄ is alkyl_{(C≤12)} or substituted alkyl_{(C≤12)}.

35. The compound according to any one of claims 1-18 and 34, wherein R₄ is substituted alkyl_{(C≤12)}.

36. The compound according to any one of claims 1-18, 34, and 35, wherein R₄ is 1-hydroxyethyl or 1-chloroethyl.

37. The compound according to any one of claims 1-18, wherein R₄ is alkenyl_{(C≤12)} or substituted alkenyl_{(C≤12)}.

38. The compound according to any one of claims 1-18 and 37, wherein R₄ is alkenyl_{(C≤12)}.

39. The compound according to any one of claims 1-18, 37, and 38, wherein R₄ is ethenyl.

40. The compound according to any one of claims 1-18, wherein R₄ is alkynyl_{(C≤12)} or substituted alkynyl_{(C≤12)}.

41. The compound according to any one of claims 1-18 and 40, wherein R₄ is alkynyl_{(C≤12)}.

42. The compound according to any one of claims 1-18, 40, and 41, wherein R₄ is propynyl.

43. The compound according to any one of claims 1-18, wherein R₄ is cycloalkylamino(C≤12) or substituted cycloalkylamino(C≤12).

44. The compound according to any one of claims 1-18 and 43, wherein R₄ is cycloalkylamino(C≤12).

45. The compound according to any one of claims 1-18, 43, and 44, wherein R₄ is cyclopropylamino, cyclopentylamino, or cyclohexylamino.

46. The compound according to any one of claims 1-18, wherein R₄ is -L₂-alkanediyl_{(C≤12)}-L₃-R₅, wherein: L₂ and L₃ is —C(O)—, —OC(O)—, —C(O)O—, —NRₐC(O)—, —C(O)NRₐ, —NRₐC(O)NHR_{a′}—, —NRₐC(S)NHR_{a′}—, —NRₐC(O)-alkanediyl_{(C≤6)}—O—, or substituted —NRₐC(O)-alkanediyl_{(C≤6)}—O—, wherein: Rₐ and Rₐ′ are each independently hydrogen, alkyl_{(C≤12)}, or substituted alkyl_{(C≤12)}; and R₅ is a ubiquitin ligase ligand or a fluorophore.

47. The compound according to any one of claims 1-18 and 46, wherein L₂ is —C(O)NRₐ—.

48. The compound according to any one of claims 1-18, 46, and 47, wherein Rₐ is hydrogen.

49. The compound according to any one of claims 1-18 and 46-48, wherein the alkanediyl_{(C≤12)} is hexanediyl.

50. The compound according to any one of claims 1-18 and 46-49, wherein L₃ is —NRₐC(S)NHR_{a′}—.

51. The compound of claim 50, wherein Rₐ or Rₐ′ is hydrogen.

52. The compound of claim 51, wherein Rₐ and Rₐ′ are both hydrogen.

53. The compound according to any one of claims 1-45, wherein the compound is further defined as: 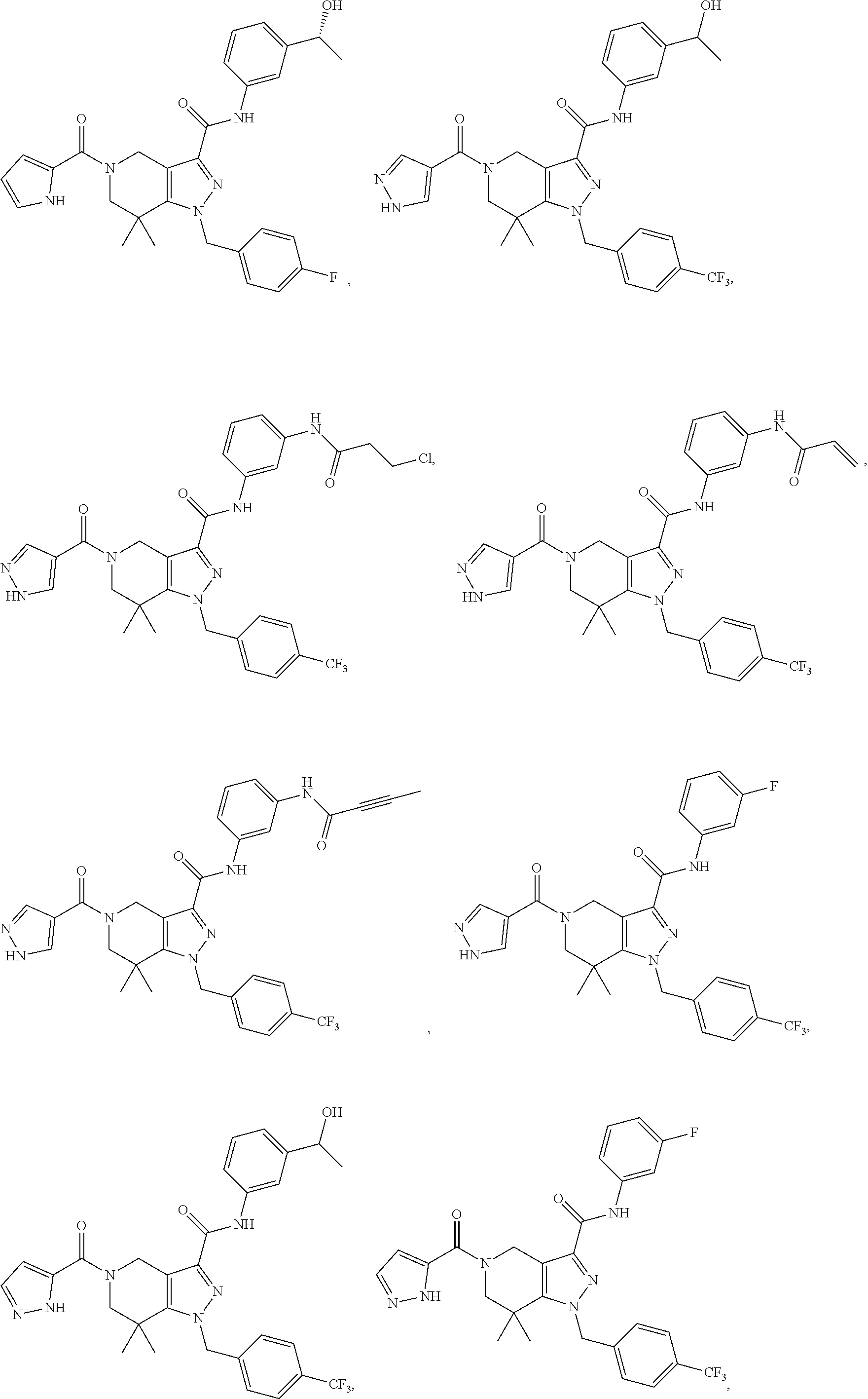 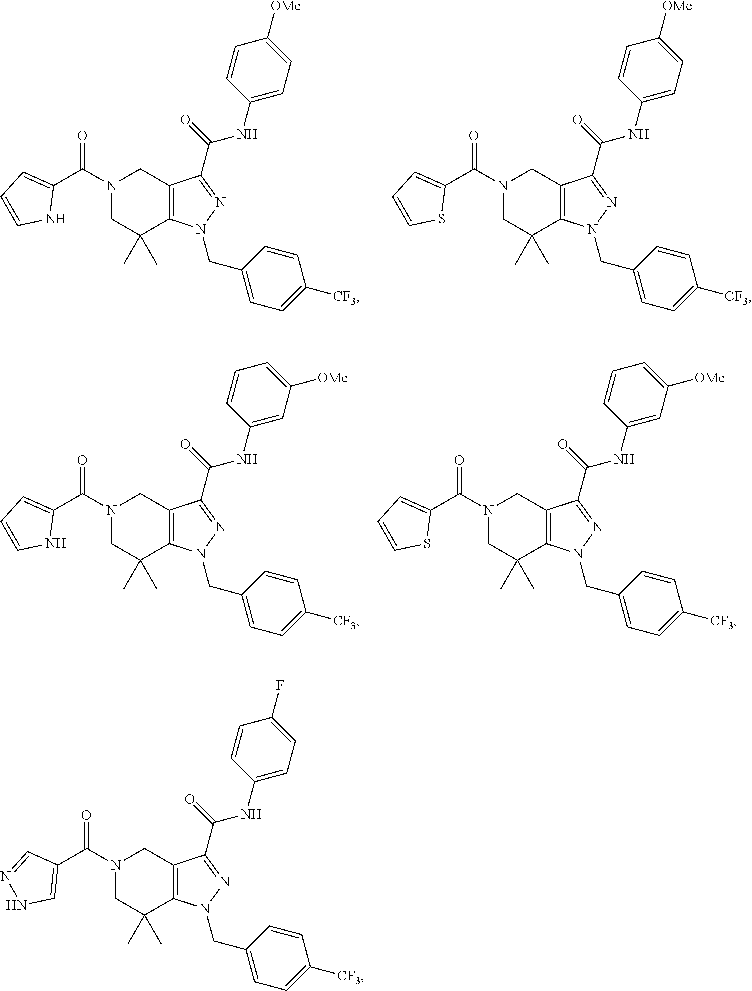 [SMILES: COc1ccc(NC(=O)c2nn(Cc3ccc(C(F)(F)F)cc3)c3c2CN(C(=O)c2ccc[nH]2)CC3(C)C)cc1 ; COc1ccc(NC(=O)c2nn(Cc3ccc(C(F)(F)F)cc3)c3c2CN(C(=O)c2cccs2)CC3(C)C)cc1 ; COc1cccc(NC(=O)c2nn(Cc3ccc(C(F)(F)F)cc3)c3c2CN(C(=O)c2ccc[nH]2)CC3(C)C)c1 ; COc1cccc(NC(=O)c2nn(Cc3ccc(C(F)(F)F)cc3)c3c2CN(C(=O)c2cccs2)CC3(C)C)c1 ; CC1(C)CN(C(=O)c2cn[nH]c2)Cc2c(C(=O)Nc3ccc(F)cc3)nn(Cc3ccc(C(F)(F)F)cc3)c21] 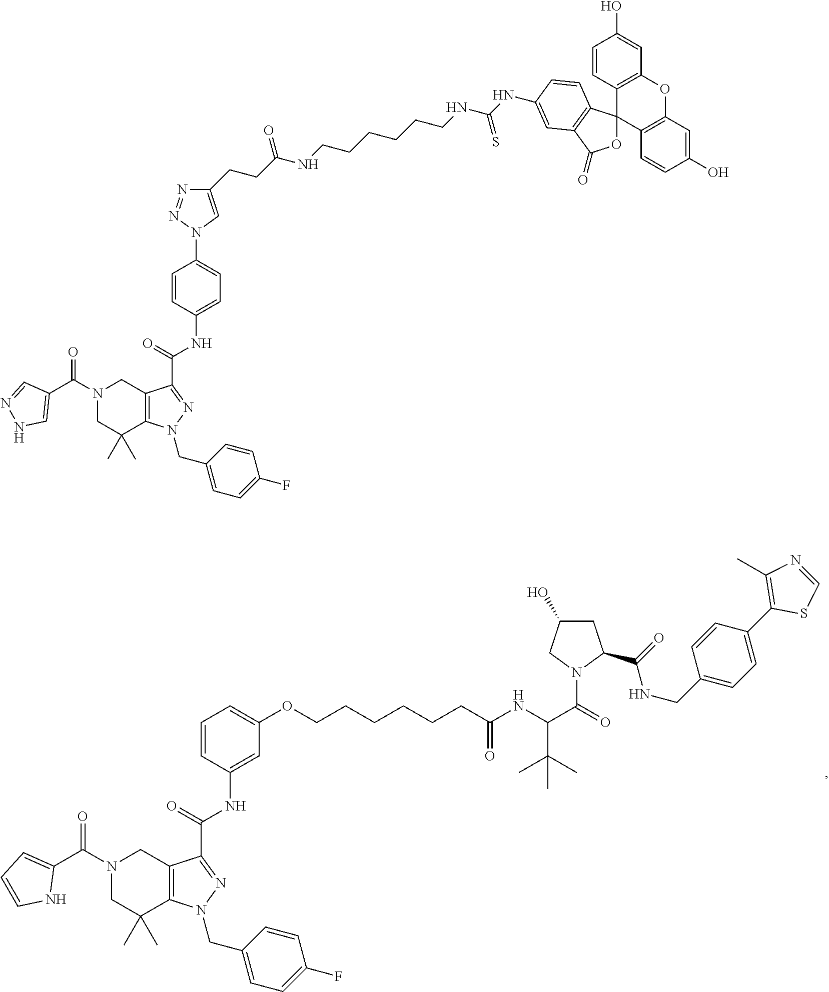 [SMILES: CC1(C)CN(C(=O)c2cn[nH]c2)Cc2c(C(=O)Nc3ccc(-n4cc(CCC(=O)NCCCCCCNC(=S)Nc5ccc6c(c5)C(=O)OC65c6ccc(O)cc6Oc6cc(O)ccc65)nn4)cc3)nn(Cc3ccc(F)cc3)c21 ; Cc1ncsc1-c1ccc(CNC(=O)[C@@H]2C[C@@H](O)CN2C(=O)C(NC(=O)CCCCCCOc2cccc(NC(=O)c3nn(Cc4ccc(F)cc4)c4c3CN(C(=O)c3ccc[nH]3)CC4(C)C)c2)C(C)(C)C)cc1] 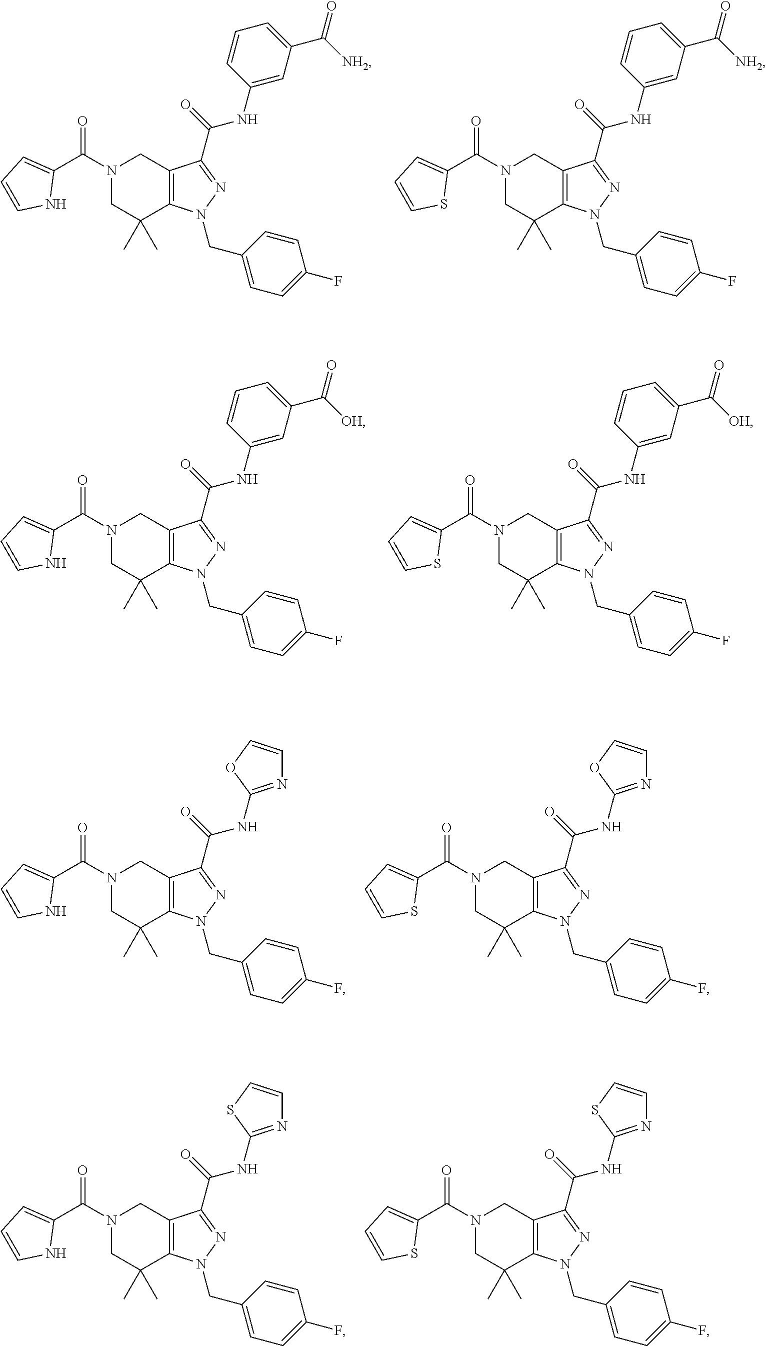 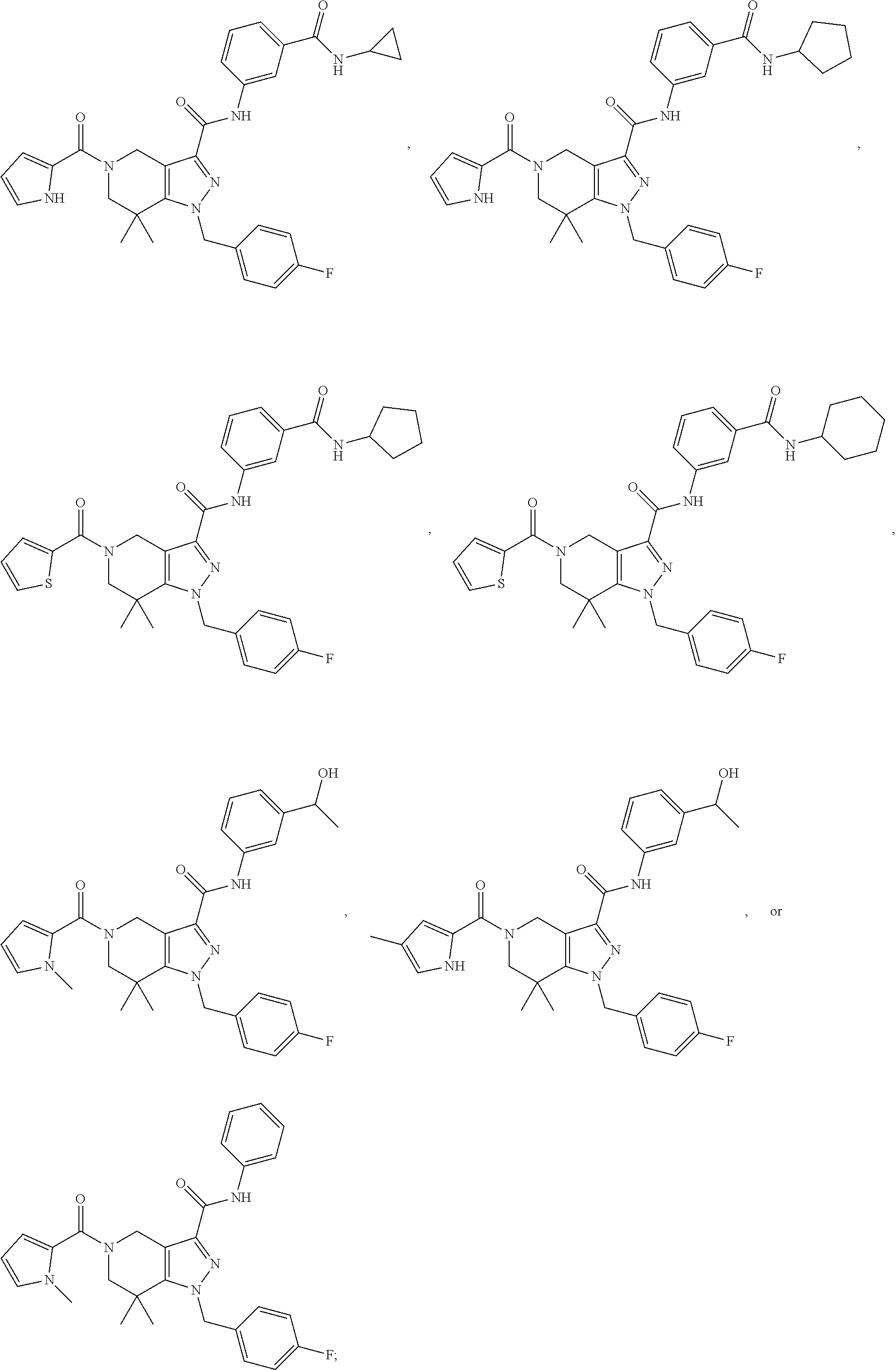
or a pharmaceutically acceptable salt thereof.

54. The compound according to any one of claims 1-53, wherein the compound is further defined as: 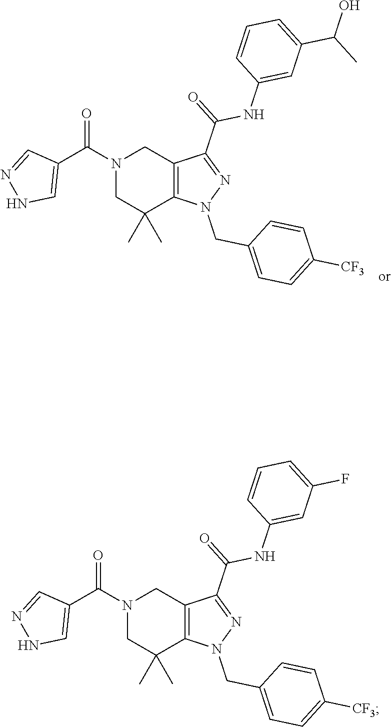 [SMILES: CC(O)c1cccc(NC(=O)c2nn(Cc3ccc(C(F)(F)F)cc3)c3c2CN(C(=O)c2cn[nH]c2)CC3(C)C)c1 ; CC1(C)CN(C(=O)c2cn[nH]c2)Cc2c(C(=O)Nc3cccc(F)c3)nn(Cc3ccc(C(F)(F)F)cc3)c21]
or a pharmaceutically acceptable salt thereof.

55. A pharmaceutical composition comprising:
a compound according to any one of claims 1-54; and
an excipient.

56. The pharmaceutical composition of claim 55, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.

57. The pharmaceutical composition of claim 56, wherein the pharmaceutical composition is formulated for oral administration.

58. The pharmaceutical composition of claim 56, wherein the pharmaceutical composition is formulated for administration via injection.

59. The pharmaceutical composition of claim 58, wherein the pharmaceutical composition is formulated for intraarterial administration, intramuscular administration, intraperitoneal administration, or intravenous administration.

60. The pharmaceutical composition according to any one of claims 55-59, wherein the pharmaceutical composition is formulated as a unit dose.

61. A method of treating a disease or disorder in a patient in need thereof comprising administering to the patient an effective amount of a compound or composition according to any one of claims 1-60.

62. The method of claim 61, wherein the patient is a mammal.

63. The method of claim 62, wherein the patient is a human.

64. The method of claim 61, wherein the disease or disorder is cancer.

65. The method of claim 64, wherein the cancer is a metastatic, recurrent, or drug-resistant cancer.

66. The method of claim 64, wherein cells of the cancer overexpress IDH1.

67. The method according to any one of claims 61-66, further comprising administering to the patient a second cancer therapy.

68. The method of claim 67, wherein the second cancer therapy is chemotherapy, immunotherapy, radiotherapy, hormone therapy, toxin therapy, or surgery.

69. The method of either claim 67 or claim 68, wherein the second cancer therapy is administered at the same time as the compound or composition.

70. The method of either claim 67 or claim 68, wherein the second cancer therapy is administered before or after the compound or composition.

71. The method of claims 61-70, further comprising administering to the patient a second administration of an effective amount of the compound or composition.

72. The method of claims 61-71, wherein the compound or composition is administered systemically, such as intravenously, intra-arterially, orally, peritoneally, subcutaneously, or by inhalation.

73. The method of claims 61-71, wherein the compound or composition is administered regionally or locally to the tumor, such as into the tumor vasculature, into a resected tumor bed, or intratumorally.

74. The method according to any one of claims 61-73, further comprising administering to the patient an effective amount of an alkali earth metal salt or an aqueous solution thereof.

75. The method of claim 74, wherein the alkali earth metal salt is a magnesium (II) salt.

76. The method of claim 75, wherein the magnesium (II) salt is MgSO₄.

77. The method of claim 74, wherein the alkali earth metal salt is administered at the same time as the compound or composition.

78. The method of either claim 67 or claim 68, wherein the second cancer therapy is administered before or after the compound or composition.

79. The method of claims 74-78, wherein the alkali earth metal salt is administered systemically, such as intravenously, intra-arterially, orally, peritoneally, or subcutaneously.

80. The method of claim 79, wherein the alkali earth metal salt is administered intravenously.

81. The method of claim 79, wherein the alkali earth metal salt is administered orally.

82. A method of inhibiting IDH1 in a cell comprising contacting the cell with a compound or composition according to any one of claims 1-60.

83. The method of claim 82, wherein the cell is a cancer cell.

84. The method of claim 82, wherein cell overexpresses IDH1.

85. A method of inhibiting IDH1 comprising contacting the enzyme with a compound or composition according to any one of claims 1-60.

86. The method of claim 85, wherein the method is performed in vitro.

87. The method of claim 85, wherein the method is performed in vivo.

88. The method of claim 85, wherein the method is performed ex vivo.


SEQUENCE LISTING (8 sequences)

SEQ ID NO: 1 - DNA, 22 bp, synthetic construct
  misc_feature 1..22: note = Synthetic oligonucleotide
  source 1..22: mol_type = other DNA
gtagatccaattccacgtaggg

SEQ ID NO: 2 - DNA, 19 bp, synthetic construct
  misc_feature 1..19: note = Synthetic oligonucleotide
  source 1..19: mol_type = other DNA
accaaatggcaccatacga

SEQ ID NO: 3 - DNA, 23 bp, synthetic construct
  misc_feature 1..23: note = Synthetic oligonucleotide
  source 1..23: mol_type = other DNA
ttcataccttgcttaatgggtgt

SEQ ID NO: 4 - DNA, 20 bp, synthetic construct
  misc_feature 1..20: note = Synthetic oligonucleotide
  source 1..20: mol_type = other DNA
attctgggtggcactgtctt

SEQ ID NO: 5 - DNA, 22 bp, synthetic construct
  misc_feature 1..22: note = Synthetic oligonucleotide
  source 1..22: mol_type = other DNA
ctctcttaagggtgtagatgcc

SEQ ID NO: 6 - DNA, 18 bp, synthetic construct
  misc_feature 1..18: note = Synthetic oligonucleotide
  source 1..18: mol_type = other DNA
gagggctagctcagaaac

SEQ ID NO: 7 - DNA, 23 bp, synthetic construct
  misc_feature 1..23: note = Synthetic oligonucleotide
  source 1..23: mol_type = other DNA
cattgtactattcttagccactg

SEQ ID NO: 8 - DNA, 19 bp, synthetic construct
  misc_feature 1..19: note = Synthetic oligonucleotide
  source 1..19: mol_type = other DNA
tggcggttctgtggtagag